(19) United States
(12) Patent Application Publication
(10) Pub. No.: US 2011/0296540 A1
(43) Pub. Date: Dec. 1, 2011

(54) ASSAY FOR SCREENING ANTIDEPRESSANTS

(76) Inventors: Rene Hen, Tenafly, NJ (US); Jingwen Wang, New york, NY (US); Eduardo David Leonardo, River Edge, NJ (US); Jesse Richardson-Jones, New York, NY (US)

(21) Appl. No.: 13/131,558

(22) PCT Filed: Dec. 1, 2009
(86) PCT No.: PCT/US09/06326

§ 371 Date: Aug. 17, 2011

Related U.S. Application Data
(60) Provisional application No. 61/200,912, filed on Dec. 3, 2008.

(51) Int. Cl. G01N 33/48; A01K 67/00; G01N 33/82; A61K 49/00; C12Q 1/68

(52) U.S. Cl. 800/3; 424/9.1; 435/6.12; 435/7.5; 800/9

(57) ABSTRACT

This invention provides a method for identifying a small molecule as an antidepressant, a method for identifying a small molecule as an anxiolytic, and a method for identifying a small molecule as able to increase dendritic arborization, decrease expression of an immaturity marker, increase expression of a maturity marker, or enhance artificial cerebrospinal fluid-type long-term potentiation in central nervous system. This invention also provides a transgenic mouse model for SSRI-non-responders.

The invention disclosed herein was made with government support National Institute of Mental Health Grant K08 MH076083 and National Institute of Mental Health Grant R01 MH068542. Accordingly, the U.S. Government has certain rights in this invention.

Throughout this application, various publications are referenced in parentheses by number. Full citations for these references may be found at the end of the specification immediately preceding the claims. The disclosures of these publications in their entireties are hereby incorporated by reference into this application to more fully describe the state of the art to which this invention pertains.

### BACKGROUND OF THE INVENTION

Selective serotonin reuptake inhibitors (SSRIs) have become the most commonly prescribed treatments for major depression (Millan, 2006). Nonetheless, the mechanisms underlying the action of antidepressants are still unclear: SSRIs require at least 2-4 weeks of administration before achieving therapeutic benefits (Wong and Licinio, 2001), despite the fact that serotonin levels rise rapidly after acute administration of SSRIs in both primates and rodents (Rutter et al., 1994; Kreiss and Lucki, 1995; Anderson et al., 2005). Due to the paradox between a rapid increase in serotonin and the delayed onset of antidepressant, it was postulated that structural or functional changes that took place over time may be required for the therapeutic effects of SSRIs.

Administration of various antidepressants increases adult neurogenesis in the dentate gyrus (DG) of the hippocampus (Malberg et al., 2000; Santarelli et al., 2003). A chronic treatment is required to produce the increase in neurogenesis (Madsen et al., 2000; Malberg et al., 2000; Santarelli et al., 2003). Additionally, it has been shown that some of the behavioral effects of SSRIs are dependent on hippocampal neurogenesis (Santarelli et al., 2003; Airan et al., 2007), indicating that neurogenesis plays a pivotal role in the mechanism of antidepressant action. Besides increasing the proliferation of neural progenitors, SSRIs have been shown to enhances survival of post-mitotic granule cells (Malberg et al., 2000; Nakagawa et al., 2002). Studies have suggested that distinct mechanisms regulate proliferation and survival. For example, environmental enrichment enhanced the survival of immature cells without affecting proliferation (Kempermann et al., 1997).

In contrast, voluntary exercise increased proliferation and survival but does not alter the rate of maturation of newborn neurons (van Praag et al., 2005; Plumpe et al., 2006). Pilocarpine-induced seizures increased both proliferation and survival (Radley and Jacobs, 2003) and also improve dendritic outgrowth in newborn neurons (Overstreet-Wadiche et al., 2006). A recent study demonstrated that fluoxetine targets a class of amplifying neural progenitors by increasing the rate of symmetric divisions (Encinas et al., 2006). It is not known if SSRIs also target immature neurons by some other mechanism.

### SUMMARY OF THE INVENTION

A method for identifying an agent as an antidepressant comprising:
a) administering the agent to a mammal for a time period of at least 14 days; and
b) determining whether adult-born neurons in the brain of the mammal exhibit (a) increased dendritic arborization, (b) decreased expression of an immaturity marker, (c) increased expression of a maturity marker, or (d) enhanced artificial cerebrospinal fluid-type long-term potentiation (ACSF-LTP) as compared to (a) dendritic arborization, (b) expression of an immaturity marker, (c) expression of a maturity marker, (d) ACSF-LTP, respectively, in a control mammal,
   wherein one or more of an increased dendritic arborization, decreased expression of an immaturity marker, increased expression of a maturity marker, or enhanced ACSF-LTP indicates that the agent is an antidepressant.

A method for identifying an agent as an anxiolytic comprising:
a) administering the agent to a mammal for a time period of at least 14 days; and
b) determining whether adult-born neurons in the brain of the mammal exhibit (a) increased dendritic arborization, (b) decreased expression of an immaturity marker, (c) increased expression of a maturity marker, or (d) enhanced artificial cerebrospinal fluid-type long-term potentiation (ACSF-LTP) as compared to (a) dendritic arborization, (b) expression of an immaturity marker, (c) expression of a maturity marker, (d) ACSF-LTP, respectively, in a control mammal,
   wherein one or more of an increased dendritic arborization, decreased expression of an immaturity marker, increased expression of a maturity marker, or enhanced ACSF-LTP indicates that the agent is an anxiolytic.

A method for identifying an agent as able to increase dendritic arborization, (b) decrease expression of an immaturity marker, (c) increase expression of a maturity marker, or (d) enhance artificial cerebrospinal fluid-type long-term potentiation (ACSF-LTP) in a central nervous system of a mammal comprising:
a) administering the agent to a mammal for a time period of at least 14 days; and
b) determining whether adult-born neurons in the brain of the mammal exhibit (a) increased dendritic arborization, (b) decreased expression of an immaturity marker, (c) increased expression of a maturity marker, or (d) enhanced artificial cerebrospinal fluid-type long-term potentiation (ACSF-LTP) as compared to (a) dendritic arborization, (b) expression of an immaturity marker, (c) expression of a maturity marker, (d) ACSF-LTP, respectively, in a control mammal,
   wherein one or more increased dendritic arborization, decreased expression of an immaturity marker, increased expression of a maturity marker, or enhanced ACSF-LTP, indicates that the agent is able to increase dendritic arborization, decrease expression of an immaturity marker, increase expression of a maturity marker, or enhance ACSF-LTP in the central nervous system of the mammal.

A method for identifying an agent as an antidepressant comprising:
a) quantitating (a) dendritic arborization, (b) expression of an immaturity marker, (c) expression of a maturity marker, or (d) artificial cerebrospinal fluid-type long-term potentiation ACSF-LTP in mammalian adult-born neurons maintained in culture;
b) contacting the neurons with the agent for a time period of at least 14 days; and
c) determining whether the neurons exhibit (a) increased dendritic arborization, (b) decreased expression of an immaturity marker, (c) increased expression of a maturity marker, or (d) enhanced ACSF-LTP,
   wherein increased dendritic arborization, decreased expression of an immaturity marker, increased expression of a maturity marker, or enhanced ACSF-LTP indicates that the agent is an antidepressant.

A method for identifying an agent as an antidepressant comprising:
a) quantitating (a) dendritic arborization, (b) expression of an immaturity marker, (c) expression of a maturity marker, or (d) artificial cerebrospinal fluid-type long-term potentiation in mammalian adult-born neurons of a hippocampal brain slice preparation;
b) contacting the neurons with the agent for a time period of at least 14 days; and
c) determining whether the neurons exhibit (a) increased dendritic arborization, (b) decreased expression of an immaturity marker, (c) increased expression of a maturity marker, or (d) enhanced ACSF-LTP,
   wherein increased dendritic arborization, decreased expression of an immaturity marker, increased expression of a maturity marker, or enhanced ACSF-LTP indicates that the agent is an antidepressant.

A method of identifying whether an agent is an antidepressant comprising administering the agent to a mammal and determining if the agent elicits an increase in an amount of beta-arrestin 2 in the brain of the mammal, wherein an increase in the amount of beta-arrestin 2 in the brain of the mammal indicates that the agent is an antidepressant.

A method of identifying whether an agent is an anxiolytic comprising administering the agent to a mammal and determining if the agent elicits an increase in an amount of beta-arrestin 2 in the brain of the mammal, wherein an increase in the amount of beta-arrestin 2 in the brain of the mammal indicates that the agent is an anxiolytic.

A method of identifying whether an agent is an antidepressant comprising administering the agent to a mammal and determining if the agent activates beta-arrestin 2 in the brain of the mammal, wherein activation of beta-arrestin 2 in the brain of the mammal indicates that the agent is an antidepressant.

A method of identifying whether an agent is an anxiolytic comprising administering the agent to a mammal and determining if the agent activates beta-arrestin 2 in the brain of the mammal, wherein activation of beta-arrestin 2 in the brain of the mammal indicates that the agent is an anxiolytic.

A mouse having a depressive phenotype, wherein the depressive phenotype results from administration of a corticosteroid to the mouse, wherein the corticosteroid is administered at a dose of 2-8 ug/kg body mass/day for a period of 14-28 days.

A transgenic mouse whose genome contains a recombinant DNA sequence comprising: (1) a DNA regulatory element operatively inserted into a promoter of an endogenous DNA sequence which encodes a human 5-hydroxytryptamine1A receptor, and (2) a serotoninergic neuron-specific promoter operatively linked to a DNA sequence encoding a tetracycline-dependent transcriptional suppressor.

A method for determining whether it is likely an agent can treat an affective disorder in a human having an affective disorder that is resistant to treatment with a selective serotonin reuptake inhibitor, which comprises: (a) quantifying a behavioral parameter which increases with the affective disorder in a transgenic mammal whose genome comprises a recombinant DNA sequence comprising: (1) a DNA regulatory element operatively inserted into a promoter of an endogenous DNA sequence which encodes a human 5-hydroxytryptamine1A receptor; and (2) a serotoninergic neuron-specific promoter operatively linked to a DNA sequence encoding a tetracycline-dependent transcriptional suppressor, wherein the transgenic mouse exhibits a depressive phenotype that is resistant to treatment with a selective serotonin reuptake inhibitor when the transgenic mammal is fed a tetracycline antibiotic, (b) administering the agent to the animal and quantifying the behavioral parameter; and (c) determining if the animal exhibits a lower level of the behavioral parameter in step c) than in step a), wherein if the animal exhibits a lower level of the behavioral parameter in step c) than in step a) then it is likely that the agent can treat the affective disorder, and wherein if the animal exhibits a higher level of the behavioral parameter in step c) than in step a) or the same amount of the behavioral parameter in step c) and step a), then it is likely that the agent cannot treat the affective disorder.

A method for determining whether it is likely an agent can treat an anxiety disorder in a human having an anxiety disorder that is resistant to treatment with a selective serotonin reuptake inhibitor, which comprises: (a) quantifying a behavioral parameter which increases with the anxiety disorder in a transgenic mammal whose genome comprises a recombinant DNA sequence comprising: (1) a DNA regulatory element operatively inserted into a promoter of an endogenous DNA sequence which encodes a human 5-hydroxytryptamine1A receptor; and (2) a serotoninergic neuron-specific promoter operatively linked to a DNA sequence encoding a tetracycline-dependent transcriptional suppressor, wherein the transgenic mouse exhibits a depressive phenotype that is resistant to treatment with a selective serotonin reuptake inhibitor when the transgenic mammal is fed a tetracycline antibiotic, (b) administering the agent to the animal and quantifying the behavioral parameter; and (c) determining if the animal exhibits a lower level of the behavioral parameter in step c) than in step a), wherein if the animal exhibits a lower level of the behavioral parameter in step c) than in step a) then it is likely that the agent can treat the anxiety disorder, and wherein if the animal exhibits a higher level of the behavioral parameter in step c) than in step a) or the same amount of the behavioral parameter in step c) and step a), then it is likely that the agent cannot treat the anxiety disorder.

A method for determining whether it is likely an agent can treat an affective disorder in a human having an affective disorder that is resistant to treatment with a selective serotonin reuptake inhibitor, which comprises: (a) quantifying a behavioral parameter which decreases with the affective disorder in a transgenic mammal whose genome comprises a recombinant DNA sequence comprising: (1) a DNA regulatory element operatively inserted into a promoter of an endogenous DNA sequence which encodes a human 5-hydroxytryptamine1A receptor; and (2) a serotoninergic neuron-specific promoter operatively linked to a DNA sequence encoding a tetracycline-dependent transcriptional suppressor, wherein the transgenic mouse exhibits a depressive phenotype that is resistant to treatment with a selective serotonin reuptake inhibitor when the transgenic mammal is fed a tetracycline antibiotic, (b) administering the agent to the animal and quantifying the behavioral parameter; and (c) determining if the animal exhibits a higher level of the behavioral parameter in step c) than in step a), wherein if the animal exhibits a higher level of the behavioral parameter in step c) than in step a) then it is likely that the agent can treat the affective disorder, and wherein if the animal exhibits a lower level of the behavioral parameter in step c) than in step a) or the same amount of the behavioral parameter in step c) and step a), then it is likely that the agent cannot treat the affective disorder.

A method for determining whether it is likely an agent can treat an anxiety disorder in a human having an anxiety disorder that is resistant to treatment with a selective serotonin reuptake inhibitor, which comprises: (a) quantifying a behavioral parameter which decreases with the anxiety disorder in a transgenic mammal whose genome comprises a recombinant DNA sequence comprising: (1) a DNA regulatory element operatively inserted into a promoter of an endogenous DNA sequence which encodes a human 5-hydroxytryptamine1A receptor; and (2) a serotoninergic neuron-specific promoter operatively linked to a DNA sequence encoding a tetracycline-dependent transcriptional suppressor, wherein the transgenic mouse exhibits a depressive phenotype that is resistant to treatment with a selective serotonin reuptake inhibitor when the transgenic mammal is fed a tetracycline antibiotic, (b) administering the agent to the animal and quantifying the behavioral parameter; and (c) determining if the animal exhibits a higher level of the behavioral parameter in step c) than in step a), wherein if the animal exhibits a higher level of the behavioral parameter in step c) than in step a) then it is likely that the agent can treat the anxiety disorder, and wherein if the animal exhibits a lower level of the behavioral parameter in step c) than in step a) or the same amount of the behavioral parameter in step c) and step a), then it is likely that the agent cannot treat the anxiety disorder.

### BRIEF DESCRIPTION OF THE FIGURES

FIGS. 1A-G Chronic but not subchronic fluoxetine treatment increased cell proliferation but not the number of DCX⁺ immature granule cells in the dentate gyrus. A, Schematic diagram of BrdU administration protocol to examine cell proliferation (n=5-6 per group). Mice were treated with vehicle (Veh), 5-6 of fluoxetine (5 d Flx), or 28 d of fluoxentine (28 d Flx). BrdU (150 mg/kg) was given 2 h before they were killed (Sac). B, The number of BrdU⁺ cells increased significantly after chronic (28 d Fix) but not subchronic (5 d Flx) fluoxetine treatment compared with vehicle-treated animals (ANOVA, F_{(2,12)}=4.11, p=0.043 for treatment). Fisher's post hoc analysis revealed significant differences between the chronic-treated group and both vehicle- and subchronic-treated groups (p<0.05). The results are mean±SEM of BrdU⁺ cells in the dentate gyrus. C, The total number of DCX⁺ cells did not change after subchronic and chronic fluoxetine treatment (ANOVA, F_{(2,12)}=0.69, p=0.52 for treatment). The results are mean±SEM of DCX⁺ cells. D-G, Images of BrdU (D, E) and DCX (F, G) immunohistochemistry after chronic fluoxetine treatment. Images were taken at 20× magnification. D, F, Vehicle-treated groups. E, G, Chronic fluoxetine-treated groups.

FIGS. 2A-D Chronic, but not subchronic fluoxetine stimulates dendritic maturation of DCX⁺ cells. A, B, Categorization of DCX⁺ immature cells. DCX⁺ cells were categorized according to their dendritic morphology into DCX⁺ cells without tertiary dendrites (A) and DCX⁺ cells with tertiary dendrites (B) (n=5-6 mice per group). C, Chronic [28 d of fluoxetine (28 d Fix)] but not subchronic fluoxetine [5 d of fluoxetine (5 d Flx)] increased the number of DCX⁺ cells with tertiary dendrites compared with vehicle (Veh)-treated animals (ANOVA, F_{(2,12)}=7.31, p=0.008 for treatment). Fisher's post hoc analysis revealed significant differences between vehicle- and chronic-treated groups (p=0.006), as well as subchronic- and chronic-treated groups (p=0.007). The results are mean±SEM of DCX⁺ cells with tertiary dendrites. D, Neither chronic nor subchronic fluoxetine changed the number of DCX⁺ cells without tertiary dendrites (ANOVA, F_{(2,12)}=0.98, p=0.40 for treatment).

FIGS. 3A-I Chronic but not subchronic fluoxetine enhances dendritic complexity of DCX⁺ cells. A, Representative image and traces from Sholl analyses of DCX⁺ cells with tertiary branches after vehicle (Veh), subchronic fluoxetine (5 d Flx), and chronic fluoxetine (28 d Flx) (n=5 mice per group, 10-12 cells per mouse). B, Chronic but not subchronic fluoxetine increased dendritic length (ANOVA, F_{(2,12)}=10.11, p=0.003 for treatment). A treatment×radius interaction (F_{(38,228)}=2.17, p<0.001) was detected. Fisher's post hoc analysis revealed significant difference between vehicle- and chronic-treated groups (*p<0.05). C, Chronic but not subchronic fluoxetine increased the number of intersections of DCX⁺ cells (ANOVA, F_{(2,12)}=9.13, p=0.004 for treatment). A treatment×radius interaction (F_{(38,228)}=1.48, p<0.001) was also detected. Fisher's post hoc analysis revealed significant difference between vehicle- and chronic-treated groups (*p<0.05). D-G, Representing images and traces from Sholl analysis of 3-week-old DCX⁺BrdU⁺ cells after 3 weeks of fluoxetine treatment. Hippocampal sections were double stained for DCX (D) and BrdU (E), and double-positive cells (F) were chosen to perform Sholl analysis on (n=5 mice per group, 4-8 cells per mouse). H, Three weeks of fluoxetine (21 d Flx) increased dendritic length compared with the vehicle group (Veh) (ANOVA, F_{(1,8)}=17.68, p=0.003). We also detected a treatment×radius interaction (F_{(1,18)}=2.68, p=0.0006). Fisher's post hoc analysis revealed significant difference between vehicle- and chronic-treated groups (*p<0.05). I, Chronic fluoxetine also increased the number of intersections (ANOVA, F_{(1,8)}=21.68, p=0.002). We also detected a treatment×radius interaction (F_{(1,18)}=2.34, p=0.003). Fisher's post hoc analysis revealed significant difference between vehicle- and chronic-treated groups (*p<0.05).

FIGS. 4A-G Chronic fluoxetine facilitates the maturation of newborn granule cells. A, Schematic diagram of BrdU administration protocol to examine survival of newborn cells (n=5-6 per group). Mice were given four BrdU injections (75 mg/kg) over 8 h on day 0. Vehicle (Veh) or fluoxetine (Flx) treatment began on day 1, 24 h after the last BrdU injection. Mice were killed 3 or 4 weeks later (Sac). B, C, Confocal images of BrdU (green), DCX (red), and NeuN (blue) immunohistochemistry. D, Chronic fluoxetine increased the number of total BrdU⁺ cells 3 and 4 weeks later compared with vehicle-treated groups (ANOVA, F_{(1,16)}=12.63, *p=0.003 for treatment; F_{(1,16)}=24.50, p<0.0001 for time). E, Chronic fluoxetine increased the number of BrdU⁺NeuN⁺ cells 3 and 4 weeks later (ANOVA, F_{(1,16)}=8.89, *p=0.01 for treatment; F_{(1,16)}=30.12, p<0.0001 for time). F, Chronic fluoxetine increased the number of BrdU⁺DCX⁻NeuN⁺ cells (ANOVA, F_{(1,14)}=30.65, *p<0.0001 for treatment; F_{(1,14)}=2.38, p=0.14 for time) but not the number of BrdU⁺DCX⁺NeuN⁺ cells (ANOVA, F_{(1,14)}=1.47×10⁻⁴, p=0.99 for treatment; F_{1,14}=62.52, p<0.0001 for time). G, Chronic fluoxetine decreased the proportion of BrdU⁺NeuN⁺ cells that are DCX⁺ (percentage of BrdU cells) but increased the proportion that are DCX⁻ (ANOVA, F_{(1,14)}=18.98, *p=0.0007 for treatment; F_{(1,14)}=132.64, p<0.0001 for time).

FIGS. 5A-H Effects of subchronic and chronic fluoxetine on hippocampal synaptic plasticity. A, B, Chronic fluoxetine (28 d Flx; B) but not subchronic fluoxetine (5 d Flx; A) reduces paired-pulse depression in both sham (Sham) and x-irradiated (x-ray) animals at stimulation intensity that elicited one-third of the maximal response compared with the vehicle group (Veh) (ANOVA, F_{(1,29)}=9.05, *p=0.005 for chronic treatment; F_{(1,29)}=0.95, p=0.34 for irradiation; F_{(1,23)}=0.17, p=0.68 for subchronic treatment; F_{(1,23)}=0.31, p=0.58 for irradiation). Inset, Representative traces of first response (1) and second response (2) (PPR, paired-pulse ratio, second response/first response). C, D, Both subchronic (C) and chronic (D) fluoxetine increased input-output relationships in both sham and x-irradiated animals (ANOVA, F_{(1,36)}=11.46, p=0.0017 in subchronic group for treatment; F_{(1,36)}=0.62, p=0.44 for irradiation; F_{(1,27)}=16.72, p=0.0003 in chronic group for treatment; F_{(1,27)}=0.23, p=0.63 for irradiation). Curves are fitted with a four-parameter logistic formula (McNaughton, 1980 [symbol] ). E, Subchronic fluoxetine suppressed ACSF-LTP, and x-irradiation completely eliminates ACSF-LTP. F, ANOVA performed on the last 10 min of LTP recording revealed a significant main effect of irradiation (F_{(1,25)}=7.28, p=0.012) as well as a main effect of subchronic fluoxetine (F_{(1,25)}=4.84, p=0.037) (F). S, Sham; X, x-irradiation, V, vehicle; F, fluoxetine; Fisher's post hoc analysis were performed between individual groups (*p<0.05). G, Chronic fluoxetine enhanced ACSF-LTP, and x-irradiation completely blocked LTP. Insets show averages of five consecutive fEPSPs at baseline (1) and in the last 10 min of LTP recordings (2). H, ANOVA performed on the last 10 min of LTP recording revealed a significant main effect of irradiation (F_{(1,27)}=63.01, p<0.0001), a main effect of chronic fluoxetine (F_{(1,27)}=4.61, p=0.041), as well as an irradiation×treatment interaction (F_{(1,27)}=6.21, p=0.019). Fisher's post hoc analysis were performed between individual groups (*p<0.05).

FIGS. 6A-D Behavioral effects of fluoxetine depend on adult neurogenesis. Novelty-suppressed feeding test on day 5 (A, B) and day 28 (C, D) of vehicle (Veh) or fluoxetine treatment. A, Five days of fluoxetine (5 d Flx) did not reduce latency to feed in sham (Sham) or x-irradiated (x-ray) animals (Cum. Survival, cumulative survival, percentage of animals that have not eaten) (Kaplan-Meier survival analysis, Mantel-Cox log-rank test, p>0.05). B, Box plot of latency to feed after 5 d of vehicle or fluoxetine. C, Twenty-eight days of fluoxetine (28 d Flx) reduced latency to feed in sham but not x-ray animals (Kaplan-Meier survival analysis, Mantel-Cox log-rank test, p=0.038 for treatment; *p<0.05 between sham fluoxetine and the other three groups; p>0.05 for all other groups). D, Box plot of latency to feed after 28 d of fluoxetine treatment. The box plot displays 10, 25, 50, 75, and 90% percentiles.

FIGS. 7A-B Chronic fluoxetine stimulates dendritic maturation and synaptic plasticity of newborn granule cells, a possible mechanism for antidepressant action. A and B, from left to right, shows anatomical and functional stages during neuronal differentiation and maturation, including quiescent, radial glia-like progenitors (green), rapidly amplifying neural progenitors (light green), immature granule cells (red), and mature granule cells. Bottom panels show immunohistochemical markers for each stage. It can be concluded from this study and others that fluoxetine stimulate adult neurogenesis in a multifold manner. Chronic fluoxetine treatment: first, increases proliferation of neural progenitors; second, stimulates dendritic branching as well as facilitates maturation; third, enhances survival of immature granule cells; fourth, enables young neurons to functionally integrate into the local hippocampal circuit, resulting in an enhancement of long-term synaptic plasticity. Finally, these synergistic actions lead to an improved behavior outcome. (Malberg et al., 2000; Encinas et al., 2006).

FIGS. 8A-H The effects of 3 weeks of antidepressant treatment was examined (IMI: imipramine, 40 mg/kg/day; FLX: fluoxetine, 18 mg/kg/day), started after 4-weeks of corticosterone (35 ug/ml/day), on anxiety behaviors in the Open-Field paradigm (A-D). Anxiety, measured by various parameters in the OF paradigm, was expressed as mean total of the time spent in the center (in seconds) for each 5 min period (A), for the entire session (B) and also for the number of entries (C). Locomotor activity was reported as ambulatory distance traveled for the entire session. Values plotted are mean±SEM (n=10-12 per group). PSLD post hoc test: **p<0.01, ##p<0.01, significant difference versus control group and corticosterone/vehicle group respectively. (E-G) Effects of chronic antidepressant treatment (IMI: imipramine, 40 mg/kg/day; FLX: fluoxetine, 18 mg/kg/day), after 7 weeks of corticosterone regimen (35 ug/ml/day), on anxiety- and depression-like behaviors in the Novelty Suppressed Feeding paradigm. Results are expressed as mean of latency to feed (in seconds) (E) or cumulative survival with percentage of animals that have not eaten over 10-min (F). The feeding drive of each mouse was assessed by returning the animal to the familiar environment of the home cage, immediately after the test, and measuring the amount of food consumed over a period of 5 min (mg/g of mouse) (F). Values plotted are mean±SEM (n=10-12 per group). PSLD post hoc test: **p<0.01, ##p<0.01, significant difference versus control group and corticosterone/vehicle group respectively; Kaplan-Meier survival analysis, Mantel-Cox log-rank test **p<0.01. (H) Effects of chronic antidepressant treatment (IMI: imipramine, 40 mg/kg/day; FLX: fluoxetine, 18 mg/kg/day) after 7 weeks of corticosterone regimen (35 ug/ml/day) on depression-like behavior in the mouse Forced Swim Test. Results are expressed as mean of immobility duration (in seconds). Values plotted are mean±SEM (n=10-12 per group). PSLD post hoc test: **p<0.01 versus control group.

FIGS. 9A-F Photos of the coat state in C57BL/6Ntac mice in controls (A) and corticosterone treated animals (B). (C) Effects of chronic antidepressant treatment (FLX: fluoxetine, 18 mg/kg/day) on corticosterone regimen induced deterioration of the coat state. Results are expressed as the total score resulting from the sum of the score of five different body parts. Values plotted are mean±SEM (n=10-12 per group). PSLD post hoc test: **p<0.01; #p<0.05 versus vehicle group and corticosterone/vehicle group respectively. (D-E) Effects of chronic antidepressant treatment (FLX: fluoxetine, 18 mg/kg/day) on corticosterone regimen (35 ug/ml/day) on anxiety- and depression related behaviors in the splash test. Results are expressed as mean grooming duration (in seconds) and frequency, measured immediately after squirting a 10% sucrose solution on the mouse's snout. Values plotted are mean±SEM (n=10-12 per group). PSLD post hoc test: **p<0.01 versus vehicle group for grooming duration and frequency parameters; #p<0.05 and ##p<0.01 versus corticosterone/vehicle group for grooming duration and frequency parameters. (F) Effects of chronic antidepressant treatment (IMI: imipramine, 40 mg/kg/day; FLX: fluoxetine, 18 mg/kg/day) on corticosterone levels after an acute stressor. Values plotted are mean±SEM (n=8-9 per group). PSLD post hoc test: **p<0.01 versus vehicle group for corticosterone levels.

FIGS. 10A-I (A) BrdU (150 mg/kg) was given 2 hours before sacrifice to examine the effects of 7 weeks of corticosterone regimen (35 ug/ml/day) with or without fluoxetine (FLX, 18 mg/kg/day) during last 3 weeks on cell proliferation. Data represent the mean±SEM of the BrdU-positive cluster counts from three to four animals per treatment group for the whole hippocampus. BrdU-positive cell counts were made within the SGZ and adjacent zone defined as a two-cell body wide zone along the hilar border (40× magnification). PSLD post hoc test: **p<0.01; #p<0.05 versus vehicle group and corticosterone/vehicle group respectively. (B) BrdU (150 mg/kg) was given twice a day during 3 days before the start of drugs treatment to examine the effect of 7 weeks of corticosterone regimen (35 ug/ml/day) with or without fluoxetine (FLX, 18 mg/kg/day) during last 3 weeks on cell survival. Data represent the mean±SEM of the BrdU-positive cells from five to six animals per treatment group. PSLD post hoc test: *p<0.05; #p<0.05 versus vehicle group and corticosterone/vehicle group respectively. (C—F) Images of doublecortin immunohistochemistry following corticosterone (35 ug/ml/day) for 7 weeks with or without chronic fluoxetine treatment (FLX, 18 mg/kg/day) for last 3 weeks. Images were taken at 20× magnification. Left panels (D and F) are vehicle treated groups. Right panels (E and G) are chronic fluoxetine treated groups. (G) The effects of fluoxetine treatment (FLX, 18 mg/kg/day) on the total number of DCX+ cells±SEM (n=4 per group) were measured after 7-weeks of corticosterone regimen (35 ug/ml/day). PSLD post hoc test: **p<0.01 versus; #p<0.05; p<0.05§ versus vehicle group; corticosterone/vehicle group and fluoxetine group respectively. (H-I) According to their dendritic morphology, DCX+ cells were categorized into DCX+ cells with or without tertiary dendrites. The effects of fluoxetine treatment (FLX, 18 mg/kg/day) on the DCX+ cells with tertiary dendrites and maturation of newborn granule cells were measured after 7-weeks of corticosterone regimen (35 ug/ml/day). Values plotted are mean±SEM (n=5 per group). PSLD **p<0.01; #p<0.05; #p<0.01; p<0.05§ versus vehicle group, corticosterone/vehicle group, fluoxetine group respectively.

FIGS. 11A-H The effects of fluoxetine (FLX, 18 mg/kg/day) treatment after focal Xirradiation of the mouse hippocampus on corticosterone (35 ug/ml/day) regimen induced anxiety-like behaviors in the Open-Field paradigm (A-D). Anxiety, measured for various parameters in the center of OF paradigm, is expressed as mean total of the time-spent (in seconds) for each 5 min period (A), for the entire session (B) and also for the number of entries (C). Locomotor activity is reported as ambulatory distance traveled for the all session. Values plotted are mean±SEM (n=10-12 per group). PSLD post hoc test: #p<0.01 versus corticosterone/vehicle group). (E-G) The effects of fluoxetine (FLX, 18 mg/kg/day) treatment after focal Xirradiation of the mouse hippocampus on corticosterone (35 ug/ml/day) regimen induced decrease of on anxiety- and depression related behaviors in the Novelty Suppressed Feeding paradigm. Results are expressed as mean of latency to feed (in seconds) (E) or cumulative survival with percentage of animals that have not eaten over 10-min (G). The feeding drive of each mouse was assessed by returning the animal to the familiar environment of the home cage immediately after the test, and measuring the amount of food consumed over a period of 5 min (mg/g of mouse) (F). Values plotted are mean±SEM (n=10-12 per group). PSLD post hoc test: (H) Effects of 3 weeks of fluoxetine treatment (FLX, 18 mg/kg/day) in 7-weeks corticosterone treated animals (35 ug/ml/day) after X-irradiation on depression-like behavior in the Forced Swim Test. Results are expressed as mean of immobility duration (in seconds). Values plotted are mean±SEM (n=10-12 per group). PSLD post hoc test: **p<0.01 versus control group.

FIGS. 12A-I The effects of fluoxetine (FLX, 18 mg/kg/day) treatment on corticosterone (35 ug/ml/day) regimen on the mean β-arrestin 1, β-arrestin 2 and Gi alpha2 gene expression (in % normalized to cyclophilin and GAPDH gene expression)±SEM (n=10-12 per group) were calculated in the mouse hypothalamus (A-C). ANOVA, Newman-Keuls post hoc test); #p<0.05 versus control group and corticosterone/vehicle group respectively. (D-F) The effects of fluoxetine (FLX, 18 mg/kg/day) treatment on corticosterone (35 ug/ml/day) regimen on the mean β-arrestin 1, β-arrestin 2 and Gi alpha2 gene expression (in % normalized to cyclophilin and GAPDH genes expression)±SEM (n=10-12 per group) were calculated in the mouse amygdala. (ANOVA, Newman-Keuls post hoc (G-I) The effects of fluoxetine (FLX, 18 mg/kg/day) treatment on corticosterone (35 ug/ml/day) regimen on the mean β-arrestin 1, β-arrestin 2 and Gi alpha2 gene expression (in % normalized to cyclophilin and GAPDH genes expression)±SEM (n=10-12 per group) were calculated in the mouse hippocampus. No statistical difference was observed between groups (ANOVA).

FIGS. 13A-H β-arrestin 2 is required for the behavioral effects of chronic fluoxetine treatment in the Open Field paradigm (A-D) and the Novelty-Suppressed Feeding test (E-G), but not in the Forced Swim test (H). (A-D) The effects of 4 weeks of fluoxetine treatment (18 mg/kg/day) was examined, in β-arrestin 2 knock-out mice (Arr2-KO) and their litermates, on anxiety behaviors in the Open-Field paradigm. Anxiety, measured by various parameters in the OF paradigm, was expressed as mean total of the time spent in the center (in seconds) for each 5 min period (A), for the entire session (B) and also for the number of entries (C). Locomotor activity was reported as ambulatory distance traveled for the entire session. Values plotted are mean±SEM (n=15-per group). PSLD post hoc test: #p<0.01, significant difference versus control group and corticosterone/vehicle group respectively. (E-G) The effects of chronic fluoxetine in β-arrestin 2 knock-out mice and their littermates in the Novelty Suppressed Feeding paradigm. Results are expressed as mean of latency to feed (in seconds) (E) or cumulative survival with percentage of animals that have not eaten over 10-min (G). The feeding drive of each mouse was assessed by returning the animal to the familiar environment of the home cage immediately after the test and measuring the amount of food consumed over a period of 5 min (mg/g of mouse) (F). Values plotted are mean±SEM (n=15-18 per group). Kaplan-Meier survival analysis, Mantel-Cox log-rank test, *p<0.05. (H) Effects of chronic antidepressant treatment in β-arrestin 2 knock-out mice and their littermates on depression-like behavior in the mouse Forced Swim Test. Results are expressed as mean of immobility duration (in seconds). Values plotted are mean±SEM (n=15-18 per group).

FIG. 14 Long-term elevations of glucocorticoid levels induce anxiety and depression-like behaviors in mice, altered progenitor cell proliferation in the hippocampus and altered gene transcription, including β-arrestins in the hypothalamus. Chronic fluoxetine reversed the behavioral and neurogenic deficits induced by chronic corticosterone, showing neurogenesis-dependent and neurogenesis-independent effects. Similarly, ablation of β-arrestin 2 blocked antidepressant-like activities in neurogenesis dependent and independent behavioral paradigms. Our findings suggest that the behavioral effects of chronic fluoxetine in the NSF and the OF paradigms in mice given chronic corticosterone require hippocampal neurogenesis and normalization of genes expression in the hypothalamus, respectively.

FIGS. 15A-B In a first set of experiments (A), in place of normal drinking water, grouped-housed male C57BL/6Ntac mice were presented during 7 weeks with vehicle (0.45% hydroxypropyl-.-cyclodextrin) or corticosterone (35 ug/ml) in the presence or absence of an antidepressant (imipramine, 40 mg/kg/day or fluoxetine, 18 mg/kg/day) during the last three weeks of the corticosterone regimen. Whether the behavioral changes during chronic corticosterone were reversed by antidepressant treatment was investigated. The same animal was successively tested in the OF paradigm, the NSF, the FST and then sacrificed for neurogenesis or transcription analysis. In another set of experiments (B), a focal X-irradiation of the hippocampus was employed to assess whether the mechanisms underlying the restoration of a normal mouse phenotype by antidepressants in corticosterone-treated animals were neurogenesis-dependent. X-radiation (5 Gy) was delivered on days 1, 4, and 8 before the start of the corticosterone treatment. All animals (Sham or X-irradiated) received 7 weeks of corticosterone (35 ug/ml) regimen in presence or absence of fluoxetine (18 mg/kg/day) during the last three weeks of the 3 weeks regimen.

FIGS. 16A-H 4 weeks corticosterone treatment (7 or 35 ug/ml per day) induced behavioral changes in the Open Field paradigm (A-D), the Novelty-Suppressed Feeding test (E-G), but not the Forced Swim test (H) in C57BL/6Ntac mice. (A-D) Effects of corticosterone (7 or 35 ug/ml/day) regimen on anxiety behaviors in the Open-Field paradigm. Anxiety, measured for various parameters in the center of OF paradigm, is expressed as mean total of the time-spent (in seconds) for each 5 min period (A), for the entire session (B) and also for the number of entries (C). Locomotor activity is reported as ambulatory distance traveled for the all session. Values plotted are mean±SEM (n=11-15 per group). PSLD post hoc test: **p<0.01 versus vehicle group). (E and G) Effects of 4 weeks of corticosterone regimen (7 or 35 ug/ml/day) on anxiety and depression related behaviors in the Novelty Suppressed Feeding paradigm. Results are expressed as mean of latency to feed (in seconds) (E) or cumulative survival with percentage of animals that have not eaten over 10-min (F). The feeding drive of each mouse was assessed by returning the animal to the familiar environment of the home cage, immediately after the test, and measuring the amount of food consumed over a period of 5 min (mg/g of mouse) (F). Values plotted are mean±SEM (n=11-15 per group). PSLD post hoc test: *p<0.05, **p<0.01 versus vehicle group) (Kaplan-Meier survival analysis, Mantel-Cox log-rank test **p<0.01). (H) Effects of 4 weeks of corticosterone regimen (35 ug/ml/day) on depression-like behavior in the Mouse Forced Swim Test. Results are expressed as mean of immobility duration (in seconds). Values plotted are mean±SEM (n=12-15 per group). No statistical difference was observed between groups.

FIGS. 17A-C 4 weeks corticosterone treatment (35 ug/ml per day) increased mouse body weight (A), food (B) and drinking consumption (C) The effects of corticosterone regimen (35 ug/ml/day) on mean mouse body weight (in g) (A), food consumption (in mg/g of mouse/day) (B) and drinking consumption (ml/g of mouse/day) (C)±SEM (n=12-15 per group) were calculated over 4-weeks of treatment. PSLD post hoc test: **p<0.01 versus control group.

FIGS. 18A-E 4 weeks corticosterone treatment (35 ug/ml per day) decreased home cage activity and flattened circadian rhythm is not reversed by chronic antidepressant treatment (A) The effects of corticosterone (35 ug/ml/day) regimen on the mean dark/total distance traveled during the light phase ratio±SEM (n=15 per group) were calculated over a hour period in the home cage. A 4-weeks corticosterone treatment flattened circadian rhythm since the dark/total distance traveled during the light phase ratio is decreased (unpaired t-test, **p<0.05). (B-C) The effects of corticosterone (35 ug/ml/day) regimen on the mean distance traveled during the dark phase (in cm) (B), during light phase (in cm) (C)±SEM (n=15 per group) were calculated over a 24 hours period in the home cage. A 4-weeks corticosterone treatment decreased ambulatory distance traveled during the dark phase but not the light phase (unpaired t-test, **p<0.05). (D) The effects of fluoxetine (FLX, 18 mg/kg/day) treatment after corticosterone (35 ug/ml/day) regimen induced decrease of the mean total distance traveled (in cm)±SEM (n=15 per group) were calculated over a 24-hours period in the home cage. PSLD post hoc test: **p<0.01 versus control group. (E) The effects of fluoxetine (FLX, 18 mg/kg/day) treatment after corticosterone (35 ug/ml/day) regimen induced increase of the inactivation duration (in seconds)±SEM (n=15 per group) were calculated over a 24-hours period in the home cage. PSLD post hoc test: **p<0.01 versus control group.

FIGS. 19A-H (A-D) Effects of corticosterone (35 ug/ml/day) regimen on anxiety behaviors in the Open-Field paradigm. Anxiety, measured for various parameters in the center of OF paradigm, is expressed as mean total of the time-spent (in seconds) for each 5 min period (A), for the entire session (B) and also for the number of entries (C). Locomotor activity is reported as ambulatory distance traveled for the all session. Values plotted are mean±SEM (n=12-15 per group). Unpaired t-test: *p<0.05 versus vehicle group). (E and G) Effects of 4-weeks of corticosterone regimen (35 ug/ml/day) on anxiety- and depression related behaviors in the Novelty Suppressed Feeding paradigm. Results are expressed as mean of latency to feed (in seconds) (E) or cumulative survival with percentage of animals that have not eaten over 10-min (F). The feeding drive of each mouse was assessed by returning the animal to the familiar environment of the home cage, immediately after the test, and measuring the amount of food consumed over a period of 5 min (mg/g of mouse) (F). Values plotted are mean±SEM (n=12-15 per group). Unpaired t-test: *p<0.05 versus vehicle group). (Kaplan-Meier survival analysis, Mantel-Cox log-rank test *p<0.05). (H) Effects of 4-weeks of corticosterone regimen (35 ug/ml/day) on depression-like behavior in the Mouse Forced Swim Test. Results are expressed as mean of immobility duration (in seconds). Values plotted are mean±SEM (n=12-15 per group). No statistical difference was observed between groups.

FIGS. 20A-F (A-B) The effects of fluoxetine (FLX, 18 mg/kg/day) treatment in combination with corticosterone (35 ug/ml/day) regimen on mean mineralocorticoid receptor (A) and Creb-1 gene (B) expression (in % cyclophilin and GAPDH genes expression)±SEM (n=10-12 per group) were calculated in the mouse hypothalamus. The levels of expression of mineralocorticoid receptor (A) and Creb-gene (B) were unchanged by chronic corticosterone alone or in combination with fluoxetine treatment. (C-D) The effects of fluoxetine (FLX, 18 mg/kg/day) treatment in combination with corticosterone (35 ug/ml/day) regimen on mean mineralocorticoid receptor (A) and Creb-1 gene (B) expression (in % cyclophilin and GAPDH genes expression)±SEM (n=10-12 per group) were calculated in the mouse amygdala. The levels of expression of mineralocorticoid receptor (C) and Creb-gene (D) were unchanged by chronic corticosterone alone or in combination with fluoxetine treatment. (E-F) The effects of fluoxetine (FLX, 18 mg/kg/day) treatment in combination with corticosterone (35 ug/ml/day) regimen on mean mineralocorticoid receptor (A) and Creb-1 gene (B) expression (in % cyclophilin and GAPDH genes expression)±SEM (n=10-12 per group) were calculated in the mouse hippocampus. The levels of expression of mineralocorticoid receptor (A) and Creb-1 gene (B) were unchanged by chronic corticosterone alone or in combination with fluoxetine treatment.

FIGS. 21A-D Transgenic mice with reversible suppression of 5-HT1ARs in the raphe. (A) Transgenic mice were created in which tTS transgene expression is driven specifically in the raphe, under the control of 40 kB of Pet-1 promoter elements. (B) This transgene was bred onto a background homozygous for Htr1atet0, in which 7 tandem tet0 DNA regulatory elements are inserted into the promoter region of the Htr1a gene. Maintenance of mice on doxycycline inhibits tTS binding and results in mice with higher expression of Htr1a in the raphe, “1A-High”. Removal of doxycycline at PND 50 for four weeks results in tTS binding to tet0 elements and suppressing Htr1a expression in the raphe, creating “1A-Low” animals. (C) Comparison of 5-HT1AR levels by 125I-MPPI-4-(2′-Methoxyphenyl)-1-[2′-(n-2″-pyridinyl)-p-[125I]iodobenzamido]ethylpiperazine) autoradiography in 1A-High and 1A-Low mice. No differences were detected in forebrain structures such as hippocampus (HPC) or entorhinal cortex (EC), while significant differences were apparent in the raphe. Detailed 5-HT1A receptor expression is shown in the dorsal (DR) and median (MR) raphe. (D) Quantification of 5-HT1A receptor levels in 1A-Low and 1A-High mice reveal significant differences in both raphe regions (n=4 mice; ***p<0.005 (DR), p<0.05 (MR) with 1-tailed t-test.

FIGS. 22A-C Functional characterization of 5-HT1A Autoreceptors in 1A-High and 1A-Low Mice. (A) 8-OH DPAT-induced hypothermia in 1A-High and 1A-Low mice. Following establishment of baseline body temperature, animals received IP injections of 0.1 mg/kg 8-OHDAPT, 0.5 mg/kg 8-OH DPAT, or saline (N=5/dose/group). Values are expressed as core temperature change from the last recorded baseline temperature. In 1A-Low mice, only the 0.5 mg/kg dose caused a significant temperature change relative to the saline control, *p=0.01. In 1A-High mice, both the 0.1 mg/kg and the 0.5 mg/kg doses elicited significantly larger temperature changes relative to control, *p=0.01 and ***p<0.0001, respectively (N=5 mice/dose/genotype). (B) Response to 5-HT1A receptor activation in 5-HT containing neurons from 1A-High and 1A-Low mice. Neurons were voltage clamped at a membrane potential of −60 mV. Downward deflections reflect spontaneous synaptic activity. Representative outward current traces in response to 100 nM 5-CT are shown. (C) Mean current elicited by 5-CT was significantly higher in 1A-High mice, p<0.01 (N=30 1A-High and 37 1A-Low neurons).

FIGS. 23A-D Baseline anxiety- and stress-related measures in 1A-High and 1A-Low animals. (A) No group differences were detected in the Open Field in either (i) Total Path, or (ii) Center Time (N=44 mice). (B) Likewise, no group differences were detected in the Light-Dark Choice Test in either (i) Time in the Light, or (ii) Total Path (N=40 mice). (C) Day 2 of the Forced Swim Test. 1A-Low mice displayed decreased immobility in the last two minutes relative to 1A-High mice, #p=0.05, and 1A-High mice displayed increasing immobility over time,*p=0.01 (N=43 mice). (D) 1A-High mice displayed a significantly attenuated Stress-Induced Hyperthermic response to novel cage stress, **p<0.01 (N=22 mice).

FIG. 24 Antidepressant response of 1A-High and 1A-Low mice to chronic fluoxetine treatment in the NSF paradigm. (i) 1A-High mice treated for 25 days with fluoxetine (18 mg/kg/day p.o.) display no difference in latency to consume a food pellet in the middle of an aversive arena than animals treated with vehicle, (N=23 mice), while (ii) 1A-Low mice treated with fluoxetine display a shorter latency to consume the pellet than vehicle-treated controls, **p<0.01 (N=21 mice).

FIGS. 25A-B Model of 5-HT1A autoreceptor effects on serotonergic raphe neurons. (A) 1A-High mice have high levels of somatodendritic 5-HT1A autoreceptor, which exert robust inhibitory effects on the raphe, as shown by 8-OH DPAT-induced hypothermia. This results in increased behavioral despair, a blunted hyperthermic response to stress, and a lack of response to chronic treatment with the SSRI fluoxetine. (B) Conversely, 1A-Low mice have low levels of somatodendritic 5-HT1A autoreceptors, which exert less inhibitory control over the raphe, as evidenced by a smaller hypothermic response to 8-OH DPAT. Decreased autoinhibition results in a robust hyperthermic response to stress, less behavioral despair, and a robust response to chronic treatment with the SSRI fluoxetine.

FIG. 26 Comparison of 5-HT1A receptor autoradiography of Pet-tTS+/tet0-1A mice on doxycycline with transgene-negative littermate. Maintenance of Pet-tTS+/tet0-1A on doxycycline (1A-High) results in complete blockade of tTS-mediate receptor suppression in the forebrain and throughout the rostrocaudal extent of the dorsal and median raphe, as visualized by 125I-labeled MPPI. Lower panel shows reference diagrams of coronal brain sections and the levels indicated, with primary areas of 5-HT1A expression shaded. (Ctx=cortex; dDG=dorsal dentate gyrus of the hippocampus; dCA1=dorsal area CA1 of the hippocampus; EC=entorhinal cortex; MR=median raphe nucleus; vDG=ventral dentate gyrus of the hippocampus; DR=dorsal raphe nucleus; Cb=cerebellum).

FIGS. 27A-C Control for the effects of doxycycline on baseline anxiety and depression-related behavior. No differences were detected in tet0-1A homozygous mice that do not carry the tTS transgene in either (A) Open Field (i) total distance, or (ii) center time (N=51 animals); (B) Light/Dark choice test (i) time in the light, or (ii) total distance (N═X).

(C) Likewise, no differences were detected in the forced swim test on either the first (i) or second (ii) day of testing (N=47).

FIG. 28 Forced Swim Test Day 1. No difference in immobility is detected between the 1A-High and 1A-Low animals on initial exposure to the forced swim test (N=43).

FIGS. 29A-B Controls for feeding motivation in the NSF test in 1A-High and 1A-Low mice. (A) No difference was detected in body weight lost between vehicle and fluoxetine treated animals after 24 hours of food deprivation. (B) No difference was detected in home cage food consumption measured over a 5 minute period immediately after testing (N=21 and 23).

FIG. 30 Hypothermic response to acute 5-HT1A agonist following chronic fluoxetine treatment. While 1A-High vehicle control mice still display a robust hypothermic response to 8-OH DPAT (0.5 mg/kg, i.p.), 1A-High mice treated with fluoxetine for 30 days show no hypothermic response. Saline-injected controls are shown for comparison. 1A-Low mice treated with fluoxetine for 30 days display a blunted hypothermic response. (N=3/drug group for each genotype).

### DETAILED DESCRIPTION OF THE INVENTION

A method for identifying an agent as an antidepressant comprising:
a) administering the agent to a mammal for a time period of at least 14 days; and
b) determining whether adult-born neurons in the brain of the mammal exhibit (a) increased dendritic arborization, (b) decreased expression of an immaturity marker, (c) increased expression of a maturity marker, or (d) enhanced artificial cerebrospinal fluid-type long-term potentiation (ACSF-LTP) as compared to (a) dendritic arborization, (b) expression of an immaturity marker, (c) expression of a maturity marker, (d) ACSF-LTP, respectively, in a control mammal,
   wherein one or more of an increased dendritic arborization, decreased expression of an immaturity marker, increased expression of a maturity marker, or enhanced ACSF-LTP indicates that the agent is an antidepressant.

A method for identifying an agent as an anxiolytic comprising:
a) administering the agent to a mammal for a time period of at least 14 days; and
b) determining whether adult-born neurons in the brain of the mammal exhibit (a) increased dendritic arborization, (b) decreased expression of an immaturity marker, (c) increased expression of a maturity marker, or (d) enhanced artificial cerebrospinal fluid-type long-term potentiation (ACSF-LTP) as compared to (a) dendritic arborization, (b) expression of an immaturity marker, (c) expression of a maturity marker, (d) ACSF-LTP, respectively, in a control mammal,
   wherein one or more of an increased dendritic arborization, decreased expression of an immaturity marker, increased expression of a maturity marker, or enhanced ACSF-LTP indicates that the agent is an anxiolytic.

A method for identifying an agent as able to increase dendritic arborization, (b) decrease expression of an immaturity marker, (c) increase expression of a maturity marker, or (d) enhance artificial cerebrospinal fluid-type long-term potentiation (ACSF-LTP) in a central nervous system of a mammal comprising:
a) administering the agent to a mammal for a time period of at least 14 days; and
b) determining whether adult-born neurons in the brain of the mammal exhibit (a) increased dendritic arborization, (b) decreased expression of an immaturity marker, (c) increased expression of a maturity marker, or (d) enhanced artificial cerebrospinal fluid-type long-term potentiation (ACSF-LTP) as compared to (a) dendritic arborization, (b) expression of an immaturity marker, (c) expression of a maturity marker, (d) ACSF-LTP, respectively, in a control mammal,
   wherein one or more increased dendritic arborization, decreased expression of an immaturity marker, increased expression of a maturity marker, or enhanced ACSF-LTP, indicates that the agent is able to increase dendritic arborization, decrease expression of an immaturity marker, increase expression of a maturity marker, or enhance ACSF-LTP in the central nervous system of the mammal.

In an embodiment of the methods the adult-born neurons are identified as such by their expression of doublecortin. In an embodiment of the methods the neurons are hippocampal granule cells. In an embodiment of the methods the dendritic arborization is quantitated by measuring the amount of tertiary branching of the dendrites of the neurons. In an embodiment of the methods the immaturity marker is doublecortin. In an embodiment of the methods the time period is at least 28 days. In an embodiment of the methods in step b) it is determined whether the agent causes increased dendritic arborization. In an embodiment of the methods in step b) it is determined whether the agent causes a decreased expression of an immaturity marker. In an embodiment of the methods in step b) it is determined whether the agent causes an increased expression of an immaturity marker. In an embodiment of the methods The method of claim 1, 2 or 3, wherein in step b) it is determined whether the agent enhances artificial cerebrospinal fluid-type long-term potentiation.

A method for identifying an agent as an antidepressant comprising:
a) quantitating (a) dendritic arborization, (b) expression of an immaturity marker, (c) expression of a maturity marker, or (d) artificial cerebrospinal fluid-type long-term potentiation ACSF-LTP in mammalian adult-born neurons maintained in culture;
b) contacting the neurons with the agent for a time period of at least 14 days; and
c) determining whether the neurons exhibit (a) increased dendritic arborization, (b) decreased expression of an immaturity marker, (c) increased expression of a maturity marker, or (d) enhanced ACSF-LTP,
   wherein increased dendritic arborization, decreased expression of an immaturity marker, increased expression of a maturity marker, or enhanced ACSF-LTP indicates that the agent is an antidepressant.

A method for identifying an agent as an antidepressant comprising:
a) quantitating (a) dendritic arborization, (b) expression of an immaturity marker, (c) expression of a maturity marker, or (d) artificial cerebrospinal fluid-type long-term potentiation in mammalian adult-born neurons of a hippocampal brain slice preparation;
b) contacting the neurons with the agent for a time period of at least 14 days; and
c) determining whether the neurons exhibit (a) increased dendritic arborization, (b) decreased expression of an immaturity marker, (c) increased expression of a maturity marker, or (d) enhanced ACSF-LTP,
   wherein increased dendritic arborization, decreased expression of an immaturity marker, increased expression of a maturity marker, or enhanced ACSF-LTP indicates that the agent is an antidepressant.

In an embodiment of the methods the mammalian adult-born neurons are identified as such by their expression of doublecortin. In an embodiment of the methods the neurons are hippocampal granule cells. In an embodiment of the methods the dendritic arborization is quantitated by measuring the amount of tertiary branching of the dendrites of the neurons. In an embodiment of the methods the immaturity marker is doublecortin. In an embodiment of the methods the time period is at least 28 days. In an embodiment of the methods the agent is a small molecule. In an embodiment of the methods the adult-born neurons are dentate gyrus neurons. In an embodiment of the methods the agent is a hydrocarbon. In an embodiment of the methods the mammal is administered a corticosteroid for 14-28 days prior to step a) of the method. In an embodiment of the methods the mammal is a non-human mammal.

A method of identifying whether an agent is an antidepressant comprising administering the agent to a mammal and determining if the agent elicits an increase in an amount of beta-arrestin 2 in the brain of the mammal, wherein an increase in the amount of beta-arrestin 2 in the brain of the mammal indicates that the agent is an antidepressant.

A method of identifying whether an agent is an anxiolytic comprising administering the agent to a mammal and determining if the agent elicits an increase in an amount of beta-arrestin 2 in the brain of the mammal, wherein an increase in the amount of beta-arrestin 2 in the brain of the mammal indicates that the agent is an anxiolytic.

A method of identifying whether an agent is an antidepressant comprising administering the agent to a mammal and determining if the agent activates beta-arrestin 2 in the brain of the mammal, wherein activation of beta-arrestin 2 in the brain of the mammal indicates that the agent is an antidepressant.

A method of identifying whether an agent is an anxiolytic comprising administering the agent to a mammal and determining if the agent activates beta-arrestin 2 in the brain of the mammal, wherein activation of beta-arrestin 2 in the brain of the mammal indicates that the agent is an anxiolytic.

In an embodiment of the instant methods the agent is a small molecule. In an embodiment of the instant methods the mammal is administered a corticosteroid for 14-28 days prior to administering the agent to the mammal. In an embodiment of the instant methods the mammal is administered 4-6 ug/kg body mass/day of the corticosteroid for 19-22 days prior to administering the agent. In an embodiment of the instant methods the mammal is a mouse or a rat. In an embodiment of the instant methods an increase in beta-arrestin 2 levels is determined by quantifying beta-arrestin 2 expression.

In an embodiment of the instant methods an increase in beta-arrestin 2 levels is determined by quantifying an increase in beta-arrestin 2-encoding mRNA levels. In an embodiment of the instant methods it is determined if the agent elicits an increase in beta-arrestin 2 levels in a hypothalamus of the brain of the mammal. In an embodiment of the instant methods an agent is an antidepressant and anxiolytic comprising administering the agent to a mammal and determining if the agent elicits an increase in beta-arrestin levels and Giα2 levels in the brain of the mammal, wherein an increase in beta-arrestin levels and Giα2 levels in the brain of the mammal indicates that the agent is an antidepressant and anxiolytic.

In an embodiment of the instant methods it is determined if the agent elicits an increase in beta-arrestin 1 and beta-arrestin 2 levels in the brain of the mammal. In an embodiment of the instant methods beta arrestin is quantified using quantitative PCR.

A mouse having a depressive phenotype, wherein the depressive phenotype results from administration of a corticosteroid to the mouse, wherein the corticosteroid is administered at a dose of 2-8 ug/kg body mass/day for a period of 14-28 days.

In an embodiment the mouse is administered the corticosteroid at a dose of 4-6 ug/kg body mass/day for a period of 18-24 days. In an embodiment the mouse is administered the corticosteroid at a dose of 5 ug/kg body mass/day for a period of 21 days. In an embodiment the mouse is a C57BL/6Ntac mouse. In an embodiment the mouse is a CD1 mouse. In an embodiment the corticosteroid is corticosterone.

A transgenic mouse whose genome contains a recombinant DNA sequence comprising: (1) a DNA regulatory element operatively inserted into a promoter of an endogenous DNA sequence which encodes a human 5-hydroxytryptamine1A receptor, and (2) a serotoninergic neuron-specific promoter operatively linked to a DNA sequence encoding a tetracycline-dependent transcriptional suppressor.

In an embodiment the transgenic mouse exhibits a depressive phenotype that is resistant to treatment with a selective serotonin reuptake inhibitor when the transgenic mouse is fed a tetracycline antibiotic. In an embodiment the tetracycline antibiotic is doxycycline. In an embodiment the DNA regulatory element comprises a tet0 DNA regulatory element.

In an embodiment the DNA regulatory element comprises seven tandem tet0 DNA regulatory elements. In an embodiment the serotoninergic neuron-specific promoter comprises a 540Z Pet-1 promoter fragment. In an embodiment the human 5-hydroxytryptamine1A receptor is UniProtKB/Swiss-Prot P08908. In an embodiment the mouse is homozygous for tet-1A and possesses a single copy of a Pet-tTS transgene. In an embodiment the mouse expresses tetracycline-dependent transcriptional suppressor in a raphe nucleus of the brain of the mouse. In an embodiment when the mouse is fed tetracycline or a tetracycline antibiotic it expresses a higher level of human 5-hydroxytryptamine1A receptor in its raphe nuclei than when the mouse is not fed a tetracycline antibiotic.

A method for determining whether it is likely an agent can treat an affective disorder in a human having an affective disorder that is resistant to treatment with a selective serotonin reuptake inhibitor, which comprises: (a) quantifying a behavioral parameter which increases with the affective disorder in a transgenic mammal whose genome comprises a recombinant DNA sequence comprising: (1) a DNA regulatory element operatively inserted into a promoter of an endogenous DNA sequence which encodes a human 5-hydroxytryptamine1A receptor; and (2) a serotoninergic neuron-specific promoter operatively linked to a DNA sequence encoding a tetracycline-dependent transcriptional suppressor, wherein the transgenic mouse exhibits a depressive phenotype that is resistant to treatment with a selective serotonin reuptake inhibitor when the transgenic mammal is fed a tetracycline antibiotic, (b) administering the agent to the animal and quantifying the behavioral parameter; and (c) determining if the animal exhibits a lower level of the behavioral parameter in step c) than in step a), wherein if the animal exhibits a lower level of the behavioral parameter in step c) than in step a) then it is likely that the agent can treat the affective disorder, and wherein if the animal exhibits a higher level of the behavioral parameter in step c) than in step a) or the same amount of the behavioral parameter in step c) and step a), then it is likely that the agent cannot treat the affective disorder.

A method for determining whether it is likely an agent can treat an anxiety disorder in a human having an anxiety disorder that is resistant to treatment with a selective serotonin reuptake inhibitor, which comprises: (a) quantifying a behavioral parameter which increases with the anxiety disorder in a transgenic mammal whose genome comprises a recombinant DNA sequence comprising: (1) a DNA regulatory element operatively inserted into a promoter of an endogenous DNA sequence which encodes a human 5-hydroxytryptamine1A receptor; and (2) a serotoninergic neuron-specific promoter operatively linked to a DNA sequence encoding a tetracycline-dependent transcriptional suppressor, wherein the transgenic mouse exhibits a depressive phenotype that is resistant to treatment with a selective serotonin reuptake inhibitor when the transgenic mammal is fed a tetracycline antibiotic, (b) administering the agent to the animal and quantifying the behavioral parameter; and (c) determining if the animal exhibits a lower level of the behavioral parameter in step c) than in step a), wherein if the animal exhibits a lower level of the behavioral parameter in step c) than in step a) then it is likely that the agent can treat the anxiety disorder, and wherein if the animal exhibits a higher level of the behavioral parameter in step c) than in step a) or the same amount of the behavioral parameter in step c) and step a), then it is likely that the agent cannot treat the anxiety disorder.

In an embodiment the transgenic mouse is fed a tetracycline antibiotic. In an embodiment the affective disorder is depression. In an embodiment the transgenic mammal is a mouse. In an embodiment the agent is a small molecule. In an embodiment the transgenic mammal is a mouse. In an embodiment the behavioral parameter associated with the affective disorder is quantified by quantifying the performance of the transgenic mammal on a forced swim test. In an embodiment the behavioral parameter is immobility. In an embodiment the behavioral parameter associated with the affective disorder is quantified by quantifying the performance of the transgenic mammal on a stress induced hyperthermia paradigm. In an embodiment the behavioral parameter is an increase in body temperature.

A method for determining whether it is likely an agent can treat an affective disorder in a human having an affective disorder that is resistant to treatment with a selective serotonin reuptake inhibitor, which comprises: (a) quantifying a behavioral parameter which decreases with the affective disorder in a transgenic mammal whose genome comprises a recombinant DNA sequence comprising: (1) a DNA regulatory element operatively inserted into a promoter of an endogenous DNA sequence which encodes a human 5-hydroxytryptamine1A receptor; and (2) a serotoninergic neuron-specific promoter operatively linked to a DNA sequence encoding a tetracycline-dependent transcriptional suppressor, wherein the transgenic mouse exhibits a depressive phenotype that is resistant to treatment with a selective serotonin reuptake inhibitor when the transgenic mammal is fed a tetracycline antibiotic, (b) administering the agent to the animal and quantifying the behavioral parameter; and (c) determining if the animal exhibits a higher level of the behavioral parameter in step c) than in step a), wherein if the animal exhibits a higher level of the behavioral parameter in step c) than in step a) then it is likely that the agent can treat the affective disorder, and wherein if the animal exhibits a lower level of the behavioral parameter in step c) than in step a) or the same amount of the behavioral parameter in step c) and step a), then it is likely that the agent cannot treat the affective disorder.

A method for determining whether it is likely an agent can treat an anxiety disorder in a human having an anxiety disorder that is resistant to treatment with a selective serotonin reuptake inhibitor, which comprises: (a) quantifying a behavioral parameter which decreases with the anxiety disorder in a transgenic mammal whose genome comprises a recombinant DNA sequence comprising: (1) a DNA regulatory element operatively inserted into a promoter of an endogenous DNA sequence which encodes a human 5-hydroxytryptamine1A receptor; and (2) a serotoninergic neuron-specific promoter operatively linked to a DNA sequence encoding a tetracycline-dependent transcriptional suppressor, wherein the transgenic mouse exhibits a depressive phenotype that is resistant to treatment with a selective serotonin reuptake inhibitor when the transgenic mammal is fed a tetracycline antibiotic, (b) administering the agent to the animal and quantifying the behavioral parameter; and (c) determining if the animal exhibits a higher level of the behavioral parameter in step c) than in step a), wherein if the animal exhibits a higher level of the behavioral parameter in step c) than in step a) then it is likely that the agent can treat the anxiety disorder, and wherein if the animal exhibits a lower level of the behavioral parameter in step c) than in step a) or the same amount of the behavioral parameter in step c) and step a), then it is likely that the agent cannot treat the anxiety disorder.

### TERMS

As used herein, and unless stated otherwise, each of the following terms shall have the definition set forth below.

“Dendritic arborization” is the extent of branching of dendrites of a neuron.

A “small molecule” is an organic molecule, which may be substituted with inorganic atoms or groups comprising inorganic atoms, which molecule has a molecular mass of less than 1000 Da.

An “antidepressant” is an agent which when administered to population of subjects suffering from a depressive disorder as set forth in the Diagnostic and Statistical Manual of Mental Disorders, Fourth Edition (DSM-IV), American Psychiatric Publishing, Inc., 1994, elicits relief from that disorder.

An “anxiolytic” is an agent which when administered to population of subjects suffering from an anxiety disorder as set forth in the Diagnostic and Statistical Manual of Mental Disorders, Fourth Edition (DSM-IV), American Psychiatric Publishing, Inc., 1994, elicits relief from that disorder.

“Artificial cerebrospinal fluid long term potentiation” is an art term which identifies the small (10% or less) long term potentiation observed in a hippocampal slice preparation perfused with artificial cerebrospinal fluid (ACSF) seen after tetanic stimulation of the afferent medial perforant pathway. The induction of ACSF-LTP is resistant to a N-methyl-D-aspartate (NMDA) receptor blocker, D,L-2-amino-5-phosphonovaleric acid (APV).

A “control” subject, e.g. a control mammal, is a subject that is administered a placebo, or vehicle, or is not administered either, but is not administered the test agent, and is a subject of the same species as the test subject. In embodiments the measured parameter from the test subject may be compared to a control parameter (instead of a control subject) which has been obtained from a population of control subjects and normalized. Thus where a method employing a control subject is performed the method can be performed mutatis mutandis comparing the quantified parameter(s) from the test subject with control parameter values.

A “maturity marker” is a detectable molecular entity, such as a protein, which is expressed by adult neurons, i.e. neurons of 4 weeks or older, in a mammalian nervous system.

An “immaturity marker” is a detectable molecular entity, such as a protein, which is primarily expressed by new-born neurons, i.e. neurons younger than 4 weeks old, rather than adult neurons in a mammalian nervous system. A non-limiting example is doublecortin.

In an embodiment of the methods described herein the corticosteroid is corticosterone.

The 5-hydroxytryptamine receptor 1A is also known as 5-HT-1A, 5-HT1A, HTR1A, and is HGNC5286, Entrez Gene 3350, Uniprot P08908 and Ensembl ENSG00000178394.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention. For example, the range 18-24 days includes 18, 19, 20, 21, 22, 23, and 24 days as well as 18.1, 18.2, 18.3, 18.4, 18.5, 18.6, 18.7, 18.8, 18.9, 19, 19.1, 19.2 . . . etc. days. For example, the range encompassed by 18-24 days includes 19-24 days, 19-23 days, 18-21 days etc.

All combinations of the various elements described herein are within the scope of the invention.

This invention will be better understood by reference to the Experimental Details which follow, but those skilled in the art will readily appreciate that the specific experiments detailed are only illustrative of the invention as described more fully in the claims which follow thereafter.

### Experimental Details

### First Series of Experiments

To assess the impact of fluoxetine on dendritic maturation, the dendritic morphology of cells that express doublecortin (DCX) was examined. In the adult DG, DCX is exclusively expressed in immature neurons from 1 d to −4 weeks of age (Brown et al., 2003; Couillard-Despres et al., 2005) and thus has been widely used as an immature neuronal marker that reliably reflects the level of neurogenesis and its modulation (Couillard-Despres et al., 2005).

Recent studies have revealed that newborn neurons display enhanced long-term potentiation (LTP) (Wang et al., 2000; Schmidt-Hieber et al., 2004; Ge et al., 2007). In addition, ACSF-LTP, a form of DGLTP induced by a weak stimulation paradigm, has been shown to be completely blocked by manipulations that ablate hippocampal neurogenesis (Snyder et al., 2001; Saxe et al., 2006). Here, it was examined whether the SSRI-induced effects on newborn neurons will lead to enhanced synaptic plasticity in the hippocampus and, finally, produce improved behavioral outcome.

Animals and drugs. SvEv129 age-matched adult male mice (12-25 weeks) were purchased from Taconic Farms (Germantown, N.Y.). Mice were housed four to five per cage in a 12 h (6:00 A. M. to 6:00 P.M.) light/dark colony room at 22° C. with available food and water ad libitum. All experiments were performed in compliance with the institutional regulations and guidelines for animal experimentation. Fluoxetine (18 mg·kg⁻¹·d⁻¹; Anawa Biomedical Services and Products, Zurich, Switzerland) was given by gavage for behavior testing or in the drinking water for all other experiments. HPLC analysis of plasma levels of fluoxetine and its metabolite norfluoxetine were determined after chronic treatment (data not shown) (Suckow et al., 1992).

Immunohistochemistry and confocal imaging. Mice were anesthetized with ketamine/xylazine (100 and 7 mg/kg, respectively) and transcardially perfused (cold saline, followed by 4% cold paraformaldehyde in PBS). All brains were postfixed overnight in 4% paraformaldehyde at 4° C., then cryoprotected in 30% sucrose, and stored at 4° C. Serial sections were cut through the entire hippocampus (Franklin and Paxinos, 1997) using a cryostat and stored in PBS. Immunohistochemistry was performed in the following steps: 2 h incubation in 1:1 formamide/2×SSC at 65° C., 5 min rinse in 2×SSC, 30 min incubation in 2N HCl at 37° C., and 10 min rinse in 0.1M boric acid, pH 8.5, 2 h incubation in 0.1M PBS with 0.3% Triton X-100, and 5% normal donkey serum. Sections were then incubated overnight at 4° C. in primary antibodies for doublecortin (goat; 1:500; Santa Cruz Biotechnology, Santa Cruz, Calif.), bromodeoxyuridine (BrdU; rat; 1:100; Serotec, Oxford, UK), and neuronal-specific nuclear protein (NeuN) (mouse; 1:500; Chemicon, Temecula, Calif.). Biotinylated or fluorescent secondary antibodies were used. All secondary antibodies were purchased from Jackson ImmunoResearch (West Grove, Pa.). DCX staining for Sholl analysis was done as follows: sections were rinsed in PBS, treated with 1% H₂O₂ in 1:1 PBS and methanol for 15 min to quench endogenous peroxidase activity (and to enhance dendritic staining), incubated in 10% normal donkey serum and 0.3% Triton X-100 for 30 min, and then incubated overnight at 4° C. in primary antibody for doublecortin. After secondary antibody incubation, sections were developed using Vector ABC kit and DAB kit. Bright-field images were taken with a Zeiss (Oberkochen, Germany) Axioplan-2 upright microscope. Stereological procedure was used to quantify labeled cells (Malberg et al., 2000 [symbol] ). All cell counting for triple-stained sections were done using a Zeiss LSM 510 META confocal microscope.

Sholl analysis. DCX-positive (DCX⁺) granule cells with tertiary, relatively untruncated dendritic branches or BrdU/DCX double-positive cells (one DCX⁺ cell was traced for each 35 hippocampal slice; n=10-12 cells per brain for DAB-stained sections; n=4-8 cells per brain for fluorescent staining, 5 mice per group) were traced using camera lucida at 40× magnification (Neurolucida; MicroBrightField, Williston, Vt.). Adult SvEvTac129 mice (16-20 weeks old) were used to obtain sparsely labeled DCX⁺ cells. DCX immunohistochemistry was done to maximize the labeling of dendrites (see above methods). Sholl analysis for dendritic complexity was performed using the accompanying software (NeuroExplorer; MicroBrightField), calculating dendritic complexity including dendritic length and number of intersections (branch points). All samples were number coded, and analysis was done blind to treatment. The dendritic complexity of DCX⁺ cells are likely to be underestimated because of the thickness of the slice (35 μm) used for DCX immunohistochemistry.

Irradiation procedure. Mice were irradiated as described previously: three times in the course of 1 week (5 Gy per day), for a cumulative dose of 15 Gy (Santarelli et al., 2003). Mice were allowed 8-12 weeks to recover from irradiation, a time after which differences in inflammation markers between sham and x-ray animals were no longer detected (Meshi et al., 2006).

Electrophysiology. Brains were collected from animals after deep anesthesia with halothane and decapitation, and transverse hippocampal slices (400 μm) were prepared using a vibratome. The slices were incubated in an interface chamber at 32° C. and perfused with oxygenated artificial CSF (in mM: 119 NaCl, 2.5 KCl, 1.3 MgSO₄, 2.5 CaCl₂, 26.2 NaHCO₃, 1 NaH₂PO₄, and 11 glucose). Slices were allowed to equilibrate for 2 h before positioning the electrodes and beginning stimulation.

To record from the DG, the medial perforant path (MPP) was stimulated using a World Precision Instruments (Sarasota, Fla.) stimulation isolation unit and a bipolar tungsten electrode. Evoked potentials were recorded in the molecular layer above the upper blade of the DG using a glass capillary microelectrode filled with artificial CSF (tip resistance of 1-3 MΩ). Isolation of the MPP was confirmed by assessing paired-pulse depression (PPD) of the MPP/DG synaptic connection at 50 ms, which generated the highest level of depression (McNaughton, 1980). Input-output curves were obtained after 10 min of stable recordings. The stimulation intensity that produced one-third of the maximal response was used for the test pulses and tetanus. After 15 min of stable baseline response to test stimulation (once every 20 s), the ability to elicit LTP was assessed. LTP was induced with a weak stimulation paradigm consisting of four trains of 1 s each, 100 Hz within the train, repeated every 15 s (Saxe et al., 2006). Responses were recorded every 20 for 60 min after LTP induction.

Novelty-suppressed feeding test. The novelty-suppressed feeding (NSF) test is a behavior paradigm that is sensitive to chronic antidepressant treatments and acute treatments with anxiolytics (such as benzodiazepines) but not subchronic antidepressant treatments (Bodnoff et al., 1989 [symbol] ). The test was performed as described previously (Santarelli et al., 2003): the testing apparatus consisted of a plastic box (50×50×20 cm). The floor was covered with ˜2 cm of wooden bedding. Twenty-four hours before behavioral testing, animals were deprived of all food in the home cage. At the time of testing, two food pellets were placed on a piece of round filter paper (12 cm diameter) positioned in the center of the box. The test began immediately after the animal was placed in a corner of the box. The latency to approach the pellet and begin feeding was recorded (maximum time, 5 min). Immediately afterward, the animal was transferred back to its home cage and the amount of food consumed in 5 min was measured. Each mouse was weighed before food deprivation and before testing to assess the percentage of body weight lost.

Statistical analysis. Data were analyzed using StatView 5.0 software (SAS Institute, Cary, N.C.). For all experiments except the novelty-suppressed feeding test, two-way ANOVA was applied to the data. Significant interactions were resolved using post hoc ANOVAs with adjusted p values. Analyses specific to each experiment are described in Results. In the novelty-suppressed feeding test, the Kaplan-Meier survival analysis was used because of the lack of normal distribution of the data. Animals that did not eat during the 5 min testing period were censored. Mantel-Cox log-rank test was used to evaluate differences between experimental groups.

### Chronic Fluoxetine Increases Cell Proliferation and Stimulates Dendritic Maturation of Newborn Cells

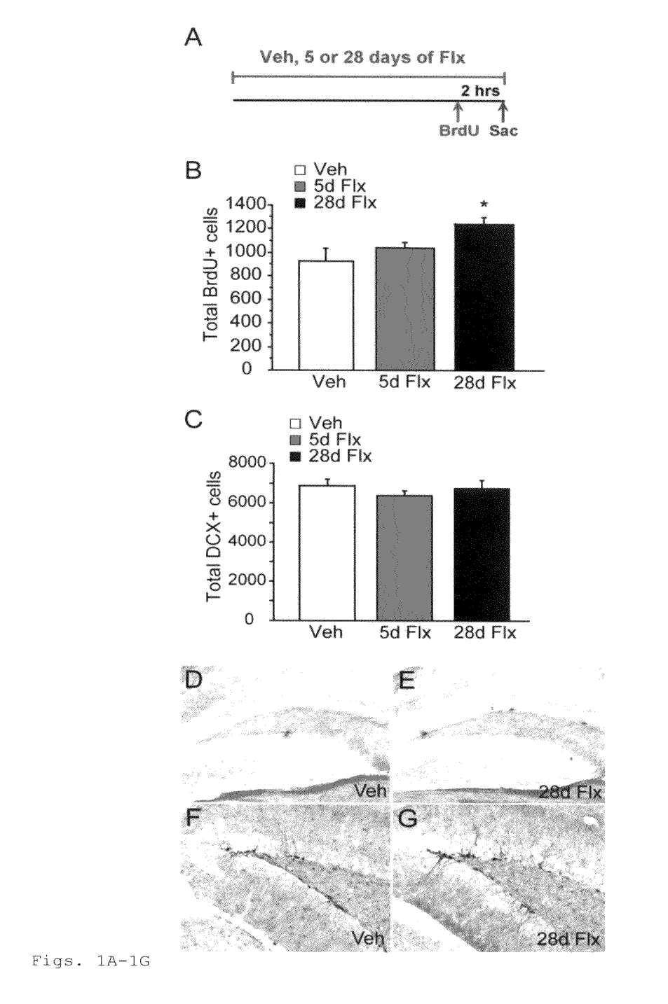

Mice were treated with vehicle, 5 d (subchronic) or 28 d (chronic) of fluoxetine. BrdU (150 mg/kg) was given 2 h before the animals were killed on the last day of treatment to label proliferating neural progenitors (FIG. 1A). Proliferation and the number of immature neurons were assessed using BrdU and DCX immunohistochemistry, respectively (FIG. 1D-G). Chronic, but not subchronic, fluoxetine treatment increased the number of BrdU cells in the granule cell layer (GCL) (F_{(2,12)}=4.11, p=0.043) (FIG. 1B). Fisher's post hoc analysis revealed significant differences between vehicle and chronic treatment groups (p=0.015). Results are mean±SEM of BrdU⁺ cells. In contrast, a change in the total number of DCX⁺ cells after chronic or subchronic fluoxetine treatments (F_{(2,12)}=0.69, p=0.52) was not detected (FIG. 1C). Results are mean±SEM of DCX⁺ cells.

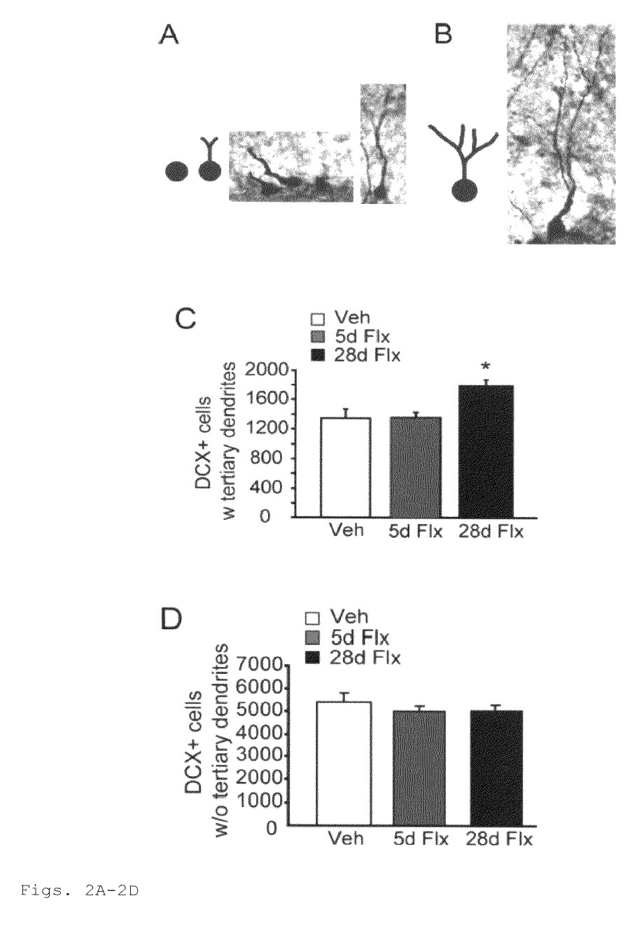

Next, the DCX⁺ cells were subcategorized according to their dendritic morphology: (1) DCX⁺ cells with no tertiary dendritic processes (FIG. 2A), and (2) DCX⁺ cells with complex, tertiary dendrites (FIG. 2B). A change in the number of DCX⁺ cells with no tertiary dendrites after either chronic or subchronic fluoxetine treatment (F_{(2,12)}=0.98, p=0.40) was not detected (FIG. 2D). However, chronic but not subchronic fluoxetine significantly increased the number of DCX⁺ cells with tertiary dendrites (F_{(2,12)}=7.31, p=0.008) (FIG. 2C). Fisher's post hoc analysis revealed significant differences between the vehicle and chronic treated groups (p=0.006), as well as between the subchronic and chronic treated groups (p=0.007). The results are mean±SEM of DCX⁺ cells with tertiary branches.

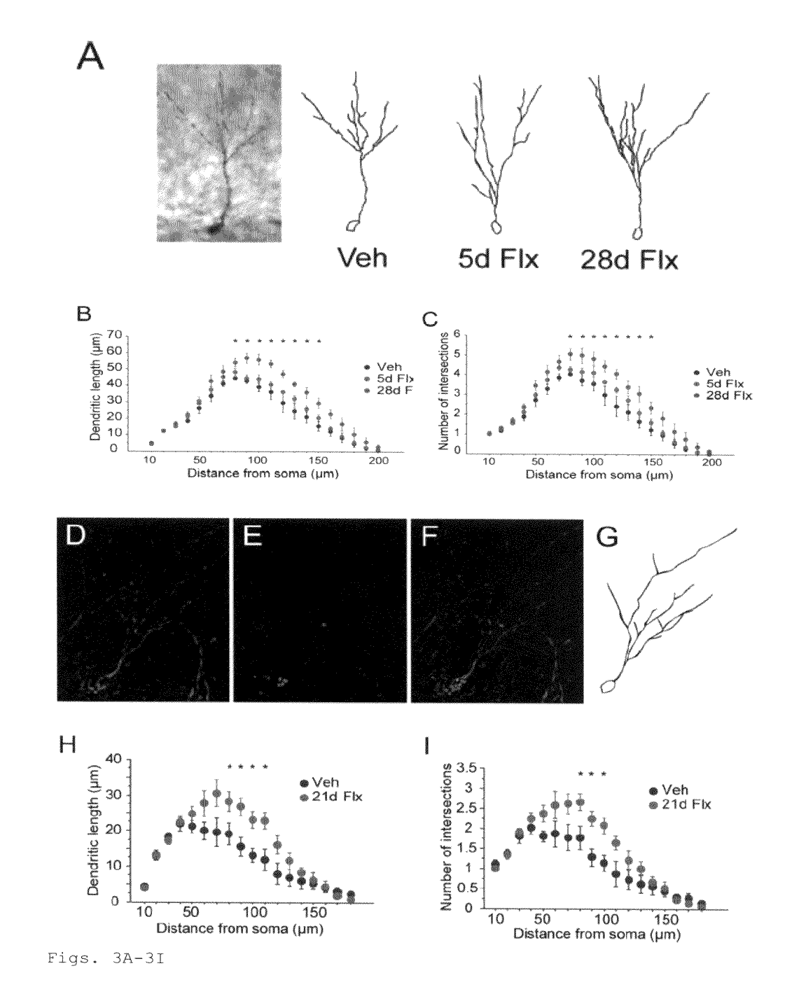

The dendrites of adult-born granule cells become progressively more complex during the 4 weeks after birth, a stage when the cells express DCX (Couillard-Despres et al., 2005). To further examine the effects of fluoxetine on the dendritic morphology of newborn cells, a Sholl analyses was performed on DCX⁺ cells with tertiary dendrites (FIG. 3A). Chronic but not subchronic fluoxetine-treated DCX⁺ cells displayed increased dendritic length (F_{(2,12)}=10.11, p=0.003) (FIG. 3B) and the number of intersections (F_{(2,12)}=9.13, p=0.004) (FIG. 3C). A significant treatment x radius interaction for both dendritic length (F_{(1,38)}=2.17, p<0.001) and number of intersections (F_{(1,38)}=1.48, p=0.043) was also detected. Fisher's post hoc analysis revealed significant differences between all groups except for vehicle and subchronic group in both dendritic length and number of intersections (p<0.05) (FIG. 3B,C).

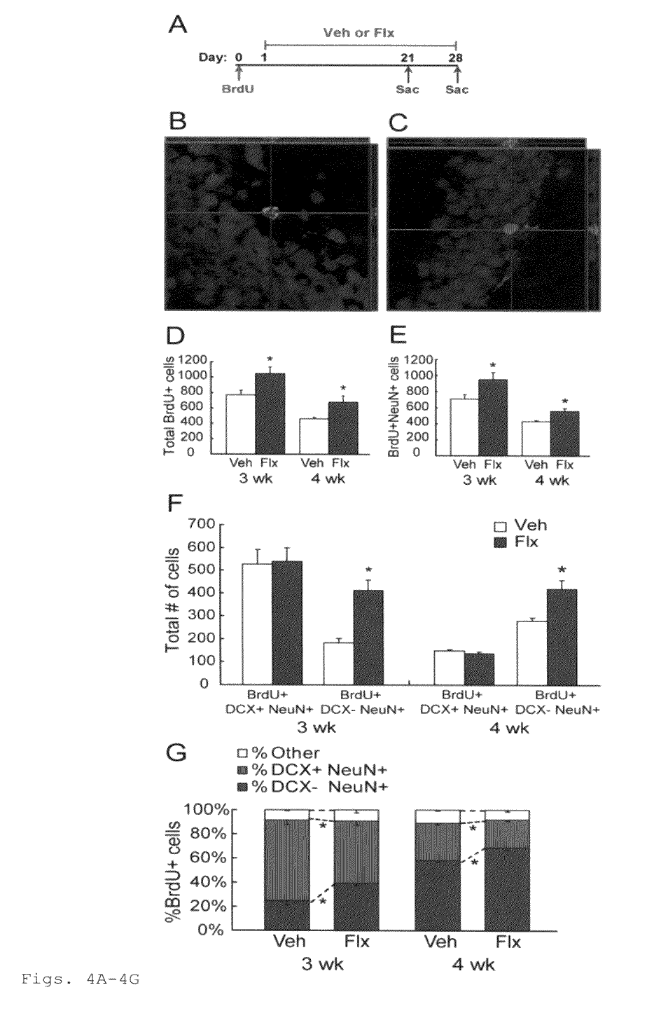

To compare dendritic morphology of DCX cells of a similar developmental stage, the animals were injected with BrdU (75 mg/kg, four times over 8 h) on day 0, were started on fluoxetine treatment on day 1, and were killed animals on day 21 (FIG. 4A). Double fluorescent immunohistochemistry for BrdU and DCX were performed on hippocampal sections. BrdU cells that were also DCX' were identified and then Sholl analysis on the double-positive cells was performed (FIG. 3 D-G). Three weeks of fluoxetine treatment, which is enough to achieve behavioral benefits in animal models of antidepressant action such as the novelty-suppressed feeding test (J.-W. Wang, unpublished data), enhanced both dendritic length and the number of intersections in 3-week-old granule cells (F_{(1,8)}=17.68, p=0.003 for dendritic length; F_{(1,8)}=21.68, p=0.002 for the number of intersections) (FIG. 3H,I). A significant treatment×radius interaction for both dendritic length (F_{(1,18)}=2.68, p=0.0006) and the number of intersections (F_{(1,18)}=2.34, p=0.003) was also detected. Fisher's post hoc analysis revealed significant differences between all groups except for vehicle and subchronic groups in both dendritic length and number of intersections (p<0.05) (FIG. 3H,I).

An alternative explanation to the increased dendritic complexity of DCX⁺ cells is that there is redistribution of DCX into dendritic processes after chronic fluoxetine. Although this is possible, other studies have demonstrated that the expression of DCX in immature granule cells is relatively stable (Couillard-Despres et al., 2005), and manipulations that either increase (voluntary exercise) or decrease (training in Morris water maze) neurogenesis do not always affect the dendritic structure of DCX cells (Couillard-Despres et al., 2005; Plumpe et al., 2006). Therefore, the former explanation is favored, which is that chronic fluoxetine stimulates dendritic maturation of newborn granule cells.

### Chronic Fluoxetine Increases Survival and Facilitates Maturation of Newborn Cells

It has been demonstrated that, after chronic fluoxetine treatment, there is an increase in cell proliferation as shown by the number of BrdU⁺ cells, but a difference was not detected in the number of immature granule cells using DCX immunohistochemistry. Two potential mechanisms may explain these seemingly paradoxical results: fluoxetine accelerates the maturation of immature cells, thereby shortening the DCX-expressing time window. In other words, newborn cells “mature/grow out of” the DCX-expressing stage faster, resulting in an unchanged number of DCX⁺ cells, or alternatively, cell death is increased in immature neurons after fluoxetine treatment, but the ones that do survive acquire more complex dendritic morphologies, thus resulting in an unchanged number of mature and immature neurons. A set of experiments was designed to test this hypothesis and to look at the effects of chronic fluoxetine on survival and maturation of newborn granule cells (FIG. 4).

As depicted in FIG. 4A, BrdU (75 mg/kg) was given four times over 8 h on day 0 to achieve maximum labeling of proliferating progenitors over a restricted time window. Fluoxetine (18 mg·kg⁻¹·d⁻¹) or vehicle administration began 24 h later and lasted for 3 or 4 weeks before the animals were killed. Hippocampal sections were triple stained for BrdU, DCX, and NeuN (FIG. 4B,C). Image acquisition and cell counting were performed using a Zeiss LSM META 510 confocal microscopy. Consistent with previous literature, both and 4 weeks of fluoxetine treatment significantly increased the total number of BrdU⁺ cells in the GCL (ANOVA, F_{(1,16)}=12.63, p=0.003 for treatment) (FIG. 4D). The total number of BrdU⁺ cells significantly decreased by ˜30% from 3 to 4 weeks after birth (ANOVA, F_{(1,16)}=24.50, p<0.0001 for time), indicating that a significant number of newborn granule cells die within 4 weeks of birth. In addition, it was found that the number of cells expressing both BrdU and the neuronal marker NeuN also increased after 3 and 4 weeks of fluoxetine (ANOVA, F_{(1,16)}=8.89, p=0.01 for treatment; F_{(1,16)}=30.12, p<0.0001 for time) (FIG. 4E), indicating that the increase in BrdU⁺ cells is mostly contributed by an increase in neurons.

The relative “maturity” of BrdU⁺NeuN⁺ cells was classified according to whether or not they express DCX (FIG. 4B,C). As expected, the number of immature BrdU⁺ granule cells (BrdU⁺NeuN⁺DCX⁺) decreased from 3 to 4 weeks after BrdU administration, indicating that the immature cells either die or progressively mature out of the DCX stage (FIG. 4F). Fluoxetine did not have an effect on the number of BrdU⁺NeuN⁺DCX⁺ cells (ANOVA, F_{(1,14)}=1.47×10⁻⁴, p=0.99 for treatment; F_{(1,14)}=62.52, p<0.0001 for time). However, the pool of “mature” BrdU⁺NeuN⁺DCX⁻ cells significantly increased after both 3 and 4 weeks of fluoxetine (ANOVA, F_{(1,14)}=30.65, p<0.0001 for treatment; F_{(1,14)}=2.38, p=0.14 for time) (FIG. 4F). These results suggest that the increase in surviving BrdU⁺NeuN⁺ cells after chronic fluoxetine treatment mostly consists of DCX⁻, mature granule cells. This result is further validated by looking at the proportion of BrdU⁺NeuN⁺ cells that are either DCX⁺ or DCX⁻ (FIG. 4G). After 3 weeks of fluoxetine treatment, the proportion of BrdU⁺NeuN⁺ cells that express DCX significantly decreased from 67.04±4.14% in the vehicle group to 51.64±3.71% in the fluoxetine group, whereas the proportion of BrdU⁺NeuN⁺ cells that ceased to express DCX significantly increased from 24.72±3.80 in the vehicle group to 39.24±1.78 in the fluoxetine group (FIG. 4G); similar effects are seen in the 4 week survival group (ANOVA, F_{(1,14)}=18.98, p=0.0007 for treatment; F_{(1,14)}=132.64, p<0.0001 for time) (FIG. 4G). An effect of fluoxetine on the proportion of BrdU⁺ cells that did not express the neuronal marker NeuN (ANOVA, F_{(1,14)}=0.235, p=0.64 for treatment; F_{(1,14)}=0.183, p=0.68 for time) was not detected, indicating that chronic fluoxetine does not change the fate determination of early progenitors.

To determine the effect of subchronic fluoxetine treatment on maturation of immature neurons another group of mice was injected with BrdU (150 mg/kg, one time) on day 0, started fluoxetine on day 1, and killed the animals on day 5. Subchronic fluoxetine treatment did not change the survival of immature neurons as measured by BrdU (F_{(1,8)}=0.22, p=0.65). In addition, 5 d of fluoxetine did not change the proportion of BrdU cells that are NeuN⁺ (F_{(1,8)}=0.047, p=0.83) or the transition of BrdU' immature cells from DCX⁺NeuN⁻ stage (F_{(1,8)}=0.039, p=0.85) to DCX⁺NeuN⁺ stage (F_{(1,8)}=0.28, p=0.61). Therefore, the results demonstrate that chronic but not subchronic fluoxetine facilitates maturation of newborn granule cells.

Chronic and Subchronic Fluoxetine have Differential Effects on Hippocampal Synaptic Plasticity

To determine whether or not the new neurons generated by chronic fluoxetine treatment functionally integrate into the local circuit and contribute to network plasticity, field electrophysiological recordings on hippocampal slices from vehicle- or fluoxetine-treated animals was performed. The previously developed focal x-irradiation protocol was used in order to completely and specifically ablate hippocampal neurogenesis (Santarelli et al., 2003). Animals were then treated with vehicle, 5 or 28 d of fluoxetine. The successful ablation using BrdU and DCX immunohistochemistry was confirmed and it was found that both the number of BrdU⁺ cells (ANOVA, F_{(1,15)}=353.85, p<0.0001) as well as DCX⁺ cells (ANOVA, F_{(1,15)}=274.80, p<0.0001) decreased dramatically after irradiation. Consistent with our previous results, an increase in the number of BrdU⁺ cells (ANOVA, F_{(1,15)}=8.17, p=0.012) was detected, but not the number of DCX⁺ cells (ANOVA, F_{(1,15)}=0.556, p=0.468) in sham animals after 28 d of fluoxetine.

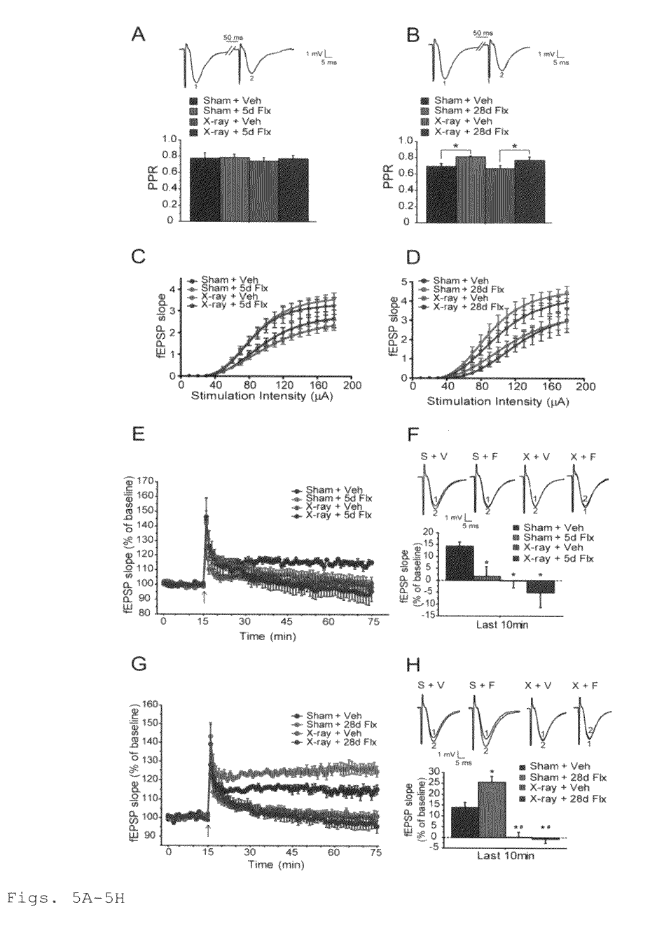

Field EPSPs (fEPSPs) were evoked by stimulating the MPP and recording in the molecular layer of the upper blade of the DG. Paired-pulse depression (50 ms interstimulus interval) was assessed to confirm that recordings were done in the medial perforant path (McNaughton, 1980). Chronic fluoxetine suppressed paired-pulse depression at stimulation intensities that generated one-third of the maximal response (ANOVA, F_{(1,29)}=9.05, p=0.005 for treatment; F_{(1,29)}=0.95, p=0.34 for irradiation) (FIG. 5B) and a constant stimulation intensity of 60 μA (ANOVA, F_{(1,27)}=7.06, p=0.013 for treatment; F_{(1,23)}=0.28, p=0.61 for irradiation) (supplemental FIG. S3, available at www.jneurosci.org as supplemental material). An effect of subchronic fluoxetine on paired-pulse depression at either stimulation intensities (ANOVA, F_{(1,23)}=0.17, p=0.68 for treatment; F_{(1,23)}=0.31, p=0.58 for irradiation at one-third of the maximum; F_{(1,23)}=0.014, p=0.91 for treatment; F_{(1,23)}=1.49, p=0.23 for irradiation at 60 μA) was not detected (FIG. 5A) (supplemental FIG. S3, available at www.jneurosci.org as supplemental material). Paired-pulse depression was not affected by x-irradiation (p>0.05). The reduced PPD after chronic fluoxetine is likely attributable to changes in either the intrinsic properties of the release process (Mennerick and Zorumski, 1995) or a feedback of glutamate onto presynaptic terminals (Brown and Reymann, 1995). Input-output relationships were then recorded. The input-output functions were fitted using a four-parameter logistic sigmoid function (DeLean et al., 1978). Both subchronic and chronic fluoxetine treatments significantly increased input-output functions in the MPP/DG (repeated-measures ANOVA, F_{(1,36)}=11.46, p=0.0017 for treatment in the subchronic group; F_{(1,27)}=16.72, p=0.0003 for treatment in the chronic group) (FIG. 5C,D). The effects of fluoxetine on input-output relationships were not sensitive to x-irradiation (ANOVA, F_{(1,36)}=0.62, p=0.44 for irradiation in the subchronic group; F_{(1,27)}=0.23, p=0.63 for irradiation in the chronic group). Although chronic treatment was required to produce the effects on PPD, fluoxetine induced rapid changes on input-output functions after only 5 d of treatment. In addition, the fluoxetine-induced effects on PPD and input-output relationships were not sensitive to x-irradiation, suggesting that these effects were not dependent on the presence of newborn neurons.

It has been previously shown that a form of long-term potentiation elicited in the MPP/DG pathway using a weak stimulation paradigm in the absence of GABA blockers (ACSF-LTP) is sensitive to manipulations that block hippocampal neurogenesis (Snyder et al., 2001; Saxe et al., 2006). It is hypothesized that, if the fluoxetine-induced new neurons functionally integrate into the local hippocampal circuit, an enhancement of synaptic plasticity as assessed by ACSF-LTP would be seen. After subchronic fluoxetine treatment, a suppression of ACSF-LTP in both sham and x-irradiated animals was observed (FIG. 5E,F). Two-way ANOVA performed on the average of the last 10 min of LTP recordings revealed a significant main effect of irradiation (F_{(1,25)}=7.28, p=0.012), a main effect of subchronic fluoxetine (F_{(1,25)}=4.84, p=0.037), but no irradiation×treatment interaction (F_{(1,25)}=0.99, p=0.33). Fisher's post hoc analysis revealed significant differences between sham vehicle group and the other three groups (sham fluoxetine, x-ray vehicle, and x-ray fluoxetine, respectively) (p<0.05). Therefore, it is conclude that the suppression of LTP by subchronic fluoxetine does not depend on neurogenesis.

After chronic treatment with fluoxetine, however, the opposite effect was seen. Chronic fluoxetine enhanced ACSF-LTP in sham animals. LTP was completely blocked in x-irradiated animals in both vehicle and chronic fluoxetine-treated groups (FIG. 5G,H). Two-way ANOVA revealed a main effect of irradiation (F_{(1,27)}=63.01, p<0.0001), a main effect of chronic fluoxetine (F_{(1,27)}=4.61, p=0.041), as well as an irradiation×treatment interaction (F_{(1,27)}=6.21, p=0.019). These results suggest that fluoxetine enhances ACSF-LTP in a time course that resembles the delayed onset of its antidepressant action. Because the fluoxetine-induced enhancing effect is not present in x-irradiated animals, it suggests that hippocampal neurogenesis is required to produce the increase in LTP. The inhibitory effects of subchronic fluoxetine on ACSF-LTP is likely the result of increased synaptic transmission that saturates the potential to further induce LTP (Stewart and Reid, 2000). However, after chronic fluoxetine treatment, increased neurogenesis and enhanced maturation of young cells may cause readjustments in the local circuitry, therefore counteracting the saturating effect and resulting in an increased ability to induce LTP, e.g., a net increase in ACSF-LTP.

### Behavioral Effects of Fluoxetine Require the Presence of Adult Neurogenesis

Do the neurogenesis-dependent effects of fluoxetine on dendritic morphology, maturation, and LTP correlate with the behavioral effects of antidepressants? Another group of animals was irradiated and the behavior after fluoxetine treatment was observed. A chronic model of antidepressant/anxiolytic action, the NSF test was used (Santarelli et al., 2003), to examine the behavioral effects of fluoxetine on days 5 and 28 of the treatment. In the NSF paradigm, conflicting motivations are produced by presenting a food-deprived animal with a reward (food) within the context of a novel, aversive environment. The NSF test is among the few behavioral paradigms that can differentiate chronic versus subchronic responses to antidepressant treatments, using the latency to begin eating as an index of antidepressant/anxiety-like behavior.

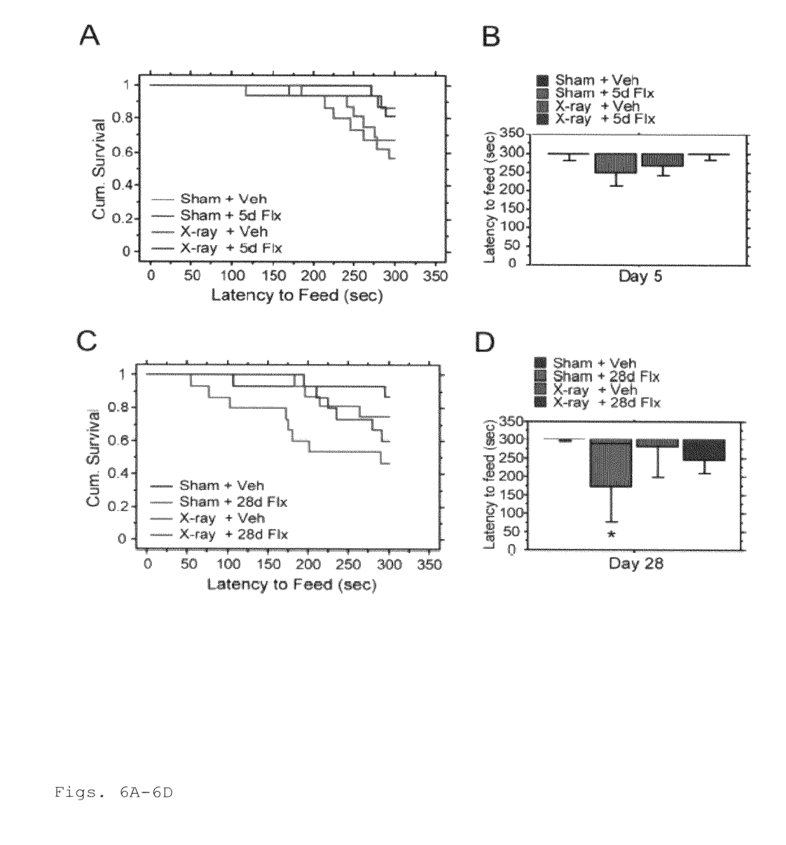

After 5 d of fluoxetine, an effect of treatment in either sham or x-irradiated animals was not detected (FIG. 6A,B) (Kaplan-Meier survival analysis was used because of a lack of normal distribution of the data, Mantel-Cox log-rank test, p=0.038 for treatment; p<0.05 between sham fluoxetine and the other three groups; p>0.05 between all other groups). Food consumption in the home cage was not different between groups (data not shown). These results indicate that chronic administration is required for the behavioral effects of fluoxetine and that neurogenesis is necessary to produce these effects. The results confirmed therefore the conclusions from recent studies showing that the behavioral effects of fluoxetine in several models of antidepressant action are dependent on adult neurogenesis (Santarelli et al., 2003; Airan et al., 2007).

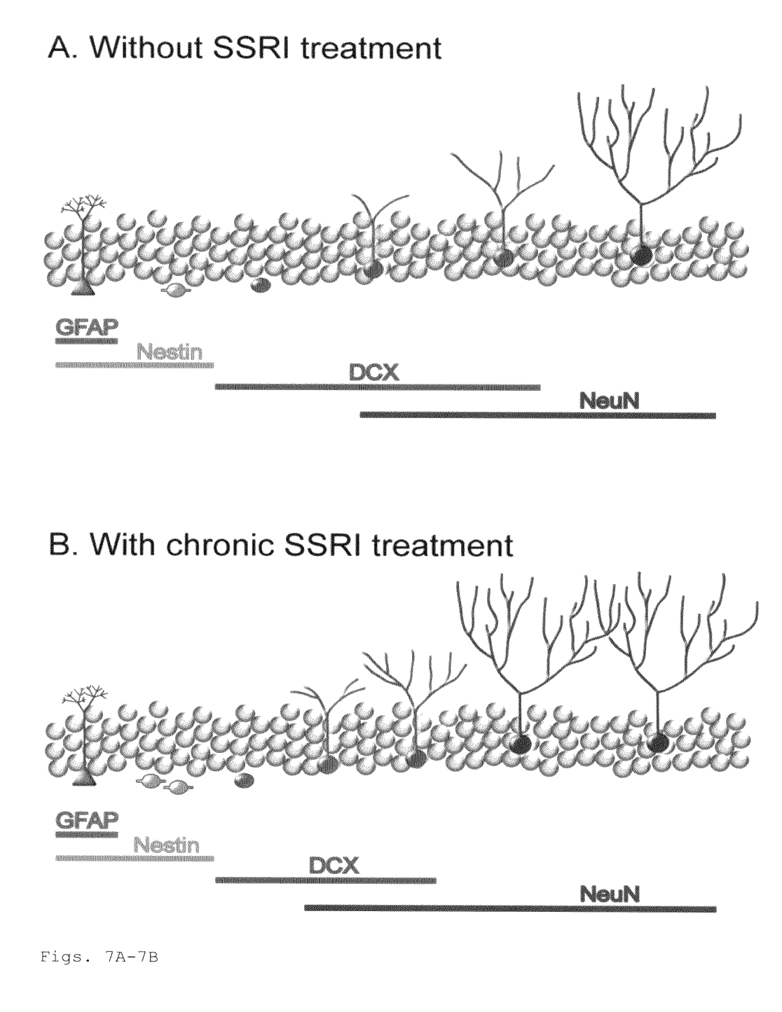

Herein it is disclosed that chronic fluoxetine increased both proliferation of progenitors and survival of immature neurons in the adult DG of the hippocampus, which is consistent with several previous studies (Malberg et al., 2000; Santarelli et al., 2003; Encinas et al., 2006). It was demonstrated for the first time that chronic but not subchronic fluoxetine stimulates maturation of immature granule cells: first, a larger fraction of DCX⁺ cells possessed tertiary dendrites after chronic fluoxetine treatment; and second, these immature, DCX⁺ cells displayed more complex dendritic arborization after chronic fluoxetine. Overall, newborn neurons undergo an accelerated maturation after chronic fluoxetine treatment, as shown by the increased proportion of newborn cells that ceased to express the immature neuronal marker DCX (FIG. 7). The delayed effects of fluoxetine to stimulate maturation of young granule cells parallel the delayed onset of its behavioral effects. Interestingly, electroconvulsive therapy (ECT), one of the fastest and most effective antidepressant treatments (American Psychiatric Association, 1990), stimulates neurogenesis more rapidly than fluoxetine (Warner-Schmidt and Duman, 2007). In addition, the induction of seizures, a prerequisite for achieving therapeutic effects during ECT (American Psychiatric Association, 1990; Sackeim et al., 1996), stimulates dendritic development and maturation (Overstreet-Wadiche et al., 2006). Specifically, after seizure induction, newborn granule cells display increased dendritic outgrowth and start receiving glutamatergic synaptic input earlier than those from non-induced animals (Overstreet-Wadiche et al., 2006). These studies together with the present results suggest that the processes that promote the maturation of newborn cells may be a target for future drug development.

### Second Series of Experiments

Understanding the physiopathology of affective disorders and their treatment relies on the availability of experimental models that accurately mimic aspects of the disease. A mouse model of an anxiety/depressive-like state induced by chronic corticosterone treatment is described here. Furthermore, chronic antidepressant treatment reversed the behavioral dysfunctions and the inhibition of hippocampal neurogenesis induced by corticosterone treatment. In corticosterone-treated mice where hippocampal neurogenesis is abolished by X-irradiation, the efficacy of fluoxetine is blocked in some but not all behavioral paradigms, suggesting both neurogenesis-dependent and independent mechanisms of antidepressant actions. Finally, a number of candidate genes, the expression of which is decreased by chronic corticosterone and normalized by chronic fluoxetine treatment selectively in the hypothalamus were identified. Importantly, mice deficient in one of these genes, β-arrestin 2, displayed a reduced response to fluoxetine in multiple tasks, suggesting β-arrestin signaling is necessary for the antidepressant effects of fluoxetine.

Despite major advances in the treatment of depression, the actions of antidepressants at the molecular and cellular level still remain poorly understood. Recently, compelling work has suggested that antidepressants exert their behavioral activity in rodents through cellular and molecular changes in the hippocampus as well as other brain structures (Santarelli et al., 2003; Airan et al., 2007; Holick et al., 2008; Surget et al., 2008, Wang et al., 2008; David et al., 2007).

The hypothalamo-pituitary-adrenal (HPA) axis, a crossroad between central and peripheral pathways, is also known to play a key role in the pathogenesis of mood disorders (Stout et al., 2002; de Kloet et al., 2005). Similarities between features of depression and disorders associated with elevated glucocorticoid levels have been reported (Sheline et al., 1996; Gould et al., 1998; McEwen et al., 1999; Airan et al., 2007; Grippo et al., 2005; Popa et al., 2008). Based on these findings, long-term exposure to exogenous corticosterone in rodents has been used to induce anxiety/depression-like changes in behavior, neurochemistry and brain morphology (Ardayfio et al., 2006; Murray et al., 2008; Gourley et al. 2008). Recently, Murray and colleagues (2008) demonstrated that behavioral deficits and decreased cell proliferation in the dentate gyrus of adult mice induced by elevation of glucocorticoid levels are reversed by chronic monoaminergic antidepressant treatment (Murray et al., 2008). In addition, in a chronic stress paradigm, the behavioral effects of some but not all antidepressants are blocked by the ablation of hippocampal neurogenesis (Surget et al., 2008).

This study modeled an anxiety/depressive-like state in mice by studying the consequences of excess glucocorticoids in an attempt to investigate both neurogenesis-dependent and independent mechanisms required for the functions of monoaminergic antidepressants. To this end, it was shown that chronic treatment with fluoxetine and imipramine in mice reversed the behavioral dysfunction induced by long-term exposure to corticosterone in the Open Field paradigm (OF), Novelty Suppressed Feeding test (NSF), Forced Swim test (FST) and splash test of grooming behavior. Chronic antidepressant treatment also stimulated the proliferation, differentiation and survival of neural progenitors in the dentate gyrus. Focal X-irradiation that ablates neurogenesis in the hippocampus while leaving other brain areas intact (Santarelli et al., 2003; David et al., 2007) coupled with behavioral tests indicates that there are neurogenesis dependent and independent mechanisms mediated by chronic fluoxetine in the model of anxiety/depression-like state.

The neurogenesis independent mechanisms underlying antidepressant efficacy may be linked to changes in signaling in brain areas other than the hippocampus, as it was shown that three genes related to G protein receptor coupling, β-arrestin 1, β-arrestin 2, and Giα2 proteins, have decreased expression in the hypothalamus that is reversed by fluoxetine. Genetic ablation of β-arrestin 2 blocked several effects of fluoxetine on behavior, suggesting that β-arrestins are necessary for the anxiolytic/antidepressant activity of fluoxetine.

### Effects of a 3-Week Antidepressant Treatment in a Novel Stress-Related Model of Anxiety/Depression.

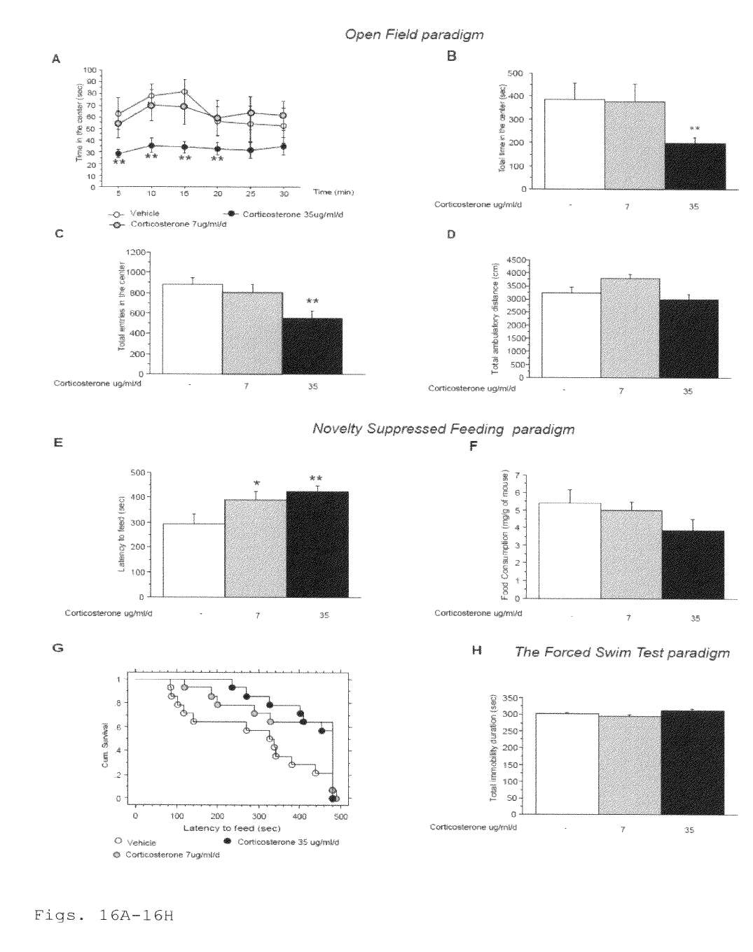

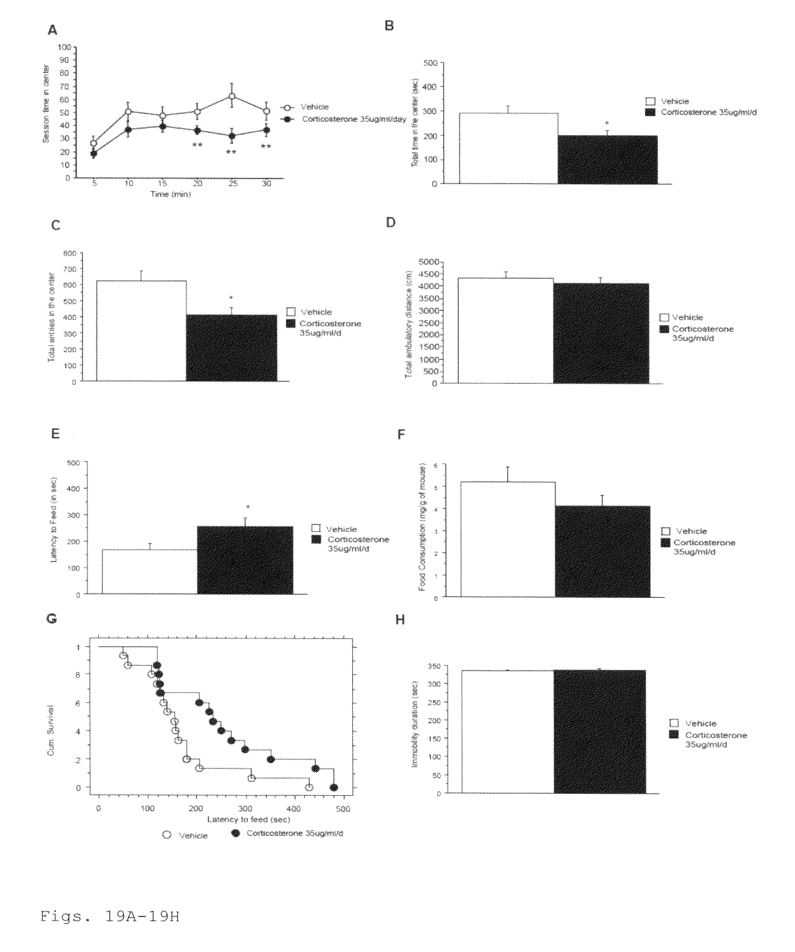

Recently, multiple studies have confirmed that long-term exposure to glucocorticoids induces anxiety and depressive-like states in rodents (Stone and Lin, 2008; Gourley et al., 2008; Murray et al., 2008). Using a low dose of corticosterone (35 ug/ml/day or 5 mg/kg/day), it was found that C57BL/6Ntac and CD1 mice treated for 4 weeks developed an anxiety-like phenotype in both the OF paradigm and the NSF test (FIGS. 16 and 19). This phenotype is not due to a locomotor deficiency since the total ambulatory distance traveled was not affected.

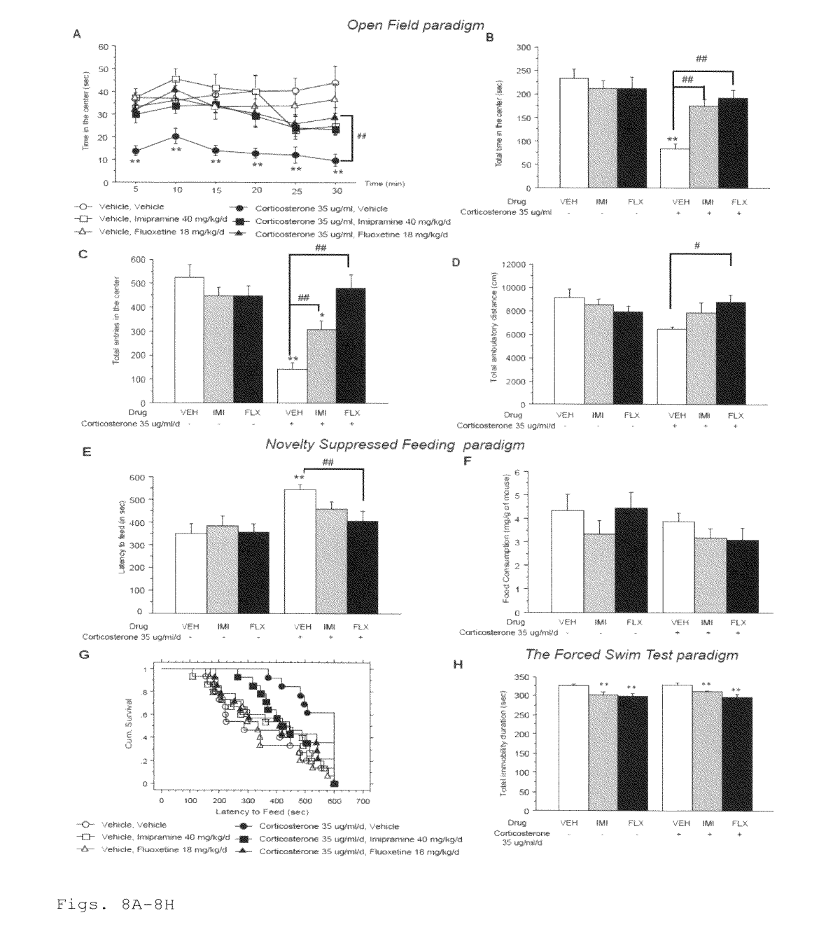

The effects of 3-week treatment of two distinct antidepressants, a tricyclic (imipramine 40 mg/kg/day) and a SSRI (fluoxetine; 18 mg/kg/day), were first tested in our model of corticosterone induced anxiety/depression-like behavior in C57BL/6Ntac mice. In the OF paradigm, chronic exogenous corticosterone had a marked effect on all anxiety parameters, resulting in decreased time spent in the center (FIG. 8A, 8B) and total number of entries in the center (8C). Interestingly, this anxiety phenotype was reversed by chronic antidepressant treatment [(two-way ANOVA **p<0.01, FIG. 8B, 8C significant effects of pretreatment, treatment factors and sampling pre-treatment x treatment interactions during the open field sessions 7 (**p<0.01); a complete statistical summary is included in Table 2)]. Regarding the ambulatory distance, chronic corticosterone treatment showed a nonsignificant trend that was abolished by chronic fluoxetine treatment (FIG. 8D).

Whether antidepressants were able to reverse the anxiety/depressive-like state observed in the NSF paradigm was then explored. Similar to the OF paradigm, the change (+36%) in the latency to feed induced by chronic corticosterone was reversed by chronic fluoxetine (18 mg/kg/day) and imipramine (40 mg/kg/day), respectively (FIG. 8E, 8G: Kaplan-Meier survival analysis, Mantel-Cox log-rank test **p<0.01), without affecting the home food consumption (FIG. 8F; two-way ANOVA, p>0.01).

In the mouse FST, two-way ANOVA revealed that chronic corticosterone had no effect, while both fluoxetine and imipramine treatment decreased the duration of immobility [FIG. 8H; significant treatment factor effect (**p<0.01)]. The decrease in immobility duration with both antidepressants was observed in corticosterone (from 328.4 s±4.2 in corticosterone group to 311.4 s±2.6 and 296.8 s±6.9 in corticosterone/fluoxetine and corticosterone/imipramine group respectively) and non-corticosterone treated animals (from 326.2 s±4.57 in vehicle to 302.9 s±6.9 and 298.5 s±6.88 in fluoxetine and imipramine group respectively).

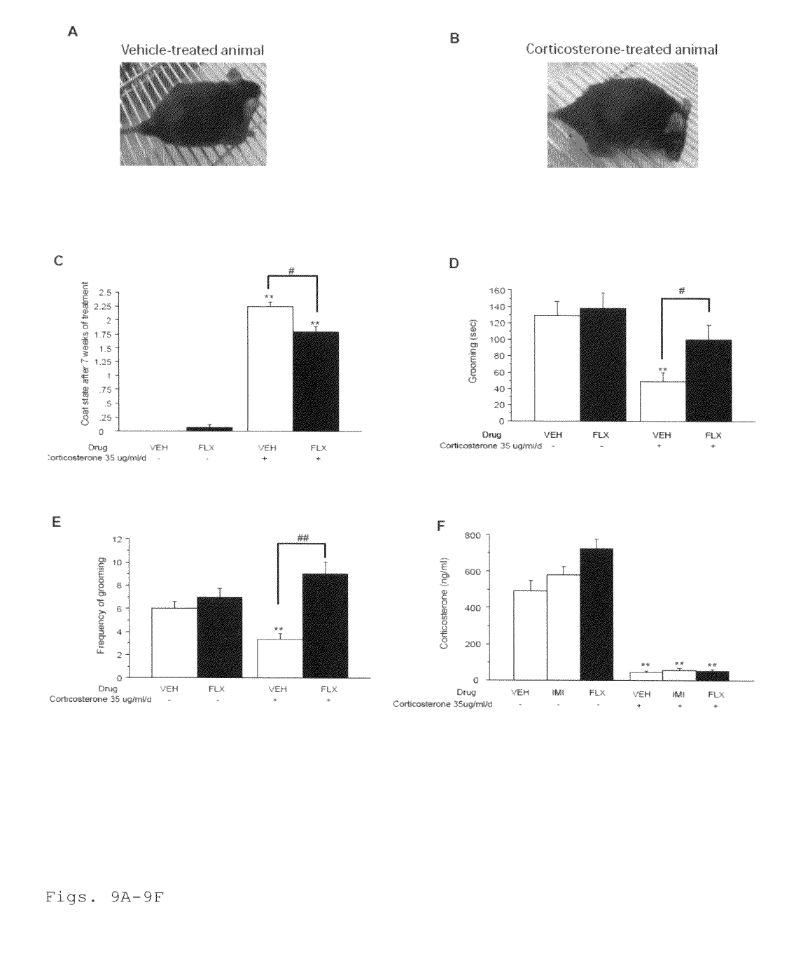

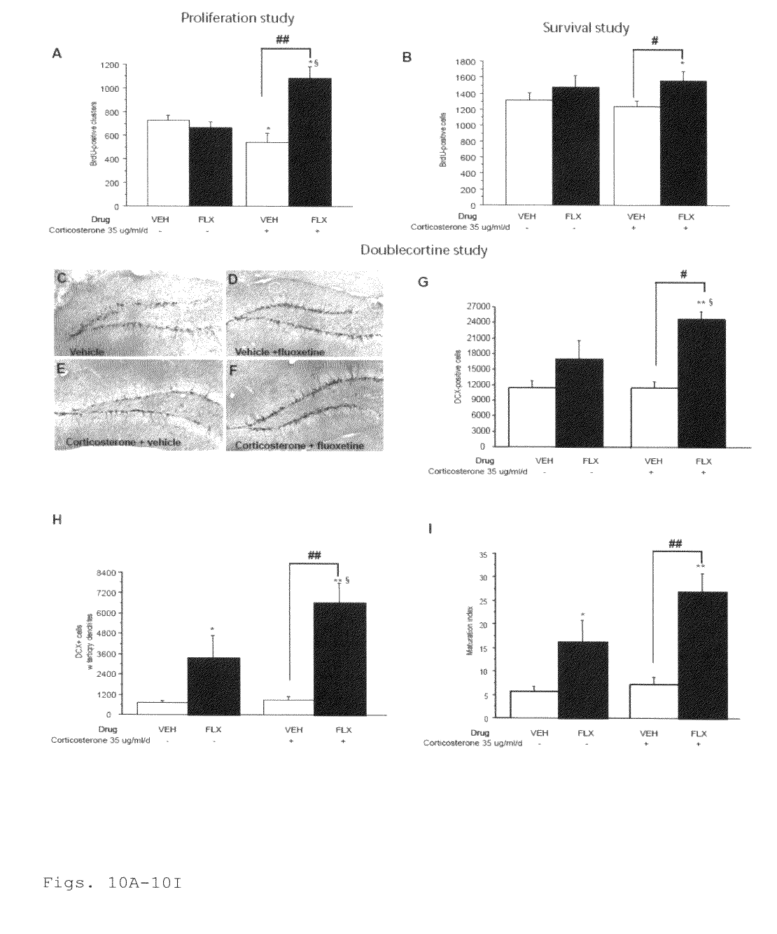

The coat state of the animals was then assessed. This measure has been described as a reliable, and well-validated index of a depressed-like state (Griebel et al., 2002; Santarelli et al., 2003; Alonso et al., 2004; Surget et al., 2008). Long-term glucocorticoid exposure, similar to chronic stress (Surget et al., 2008), induced physical changes including deterioration of coat state (FIG. 9A) and altered body weight (supplemental FIG. 10A). It was found that a 3 week fluoxetine regimen reversed the deterioration of the coat state (FIG. 2C) induced by chronic corticosterone (from 2.23±0.09 to 1.80±0.08) [two-way ANOVA with significant effect of pre-treatment, treatment factors and sampling pre-treatment x treatment interactions (**p<0.01)]. It was then investigated whether the deterioration of the coat state was linked to changes in grooming behavior (FIG. 9D). It was observed that after squirting a 10% sucrose solution on the mouse's snout, the decreased grooming duration (−63%) and frequency (−55% induced by corticosterone treatment was reversed with 3 weeks of fluoxetine treatment (18 mg/kg/day) (from 48.7 s±11.2 to 99.8 s±18.3 and from 3.3±0.5 to 9±1 for grooming duration and frequency, respectively) [two-way ANOVA with significant effect of treatment factor, pre-treatment×treatment interactions for grooming duration and significant pretreatment factor for frequency of grooming (**p<0.01)]. Taken together, these results suggest through multiple behavioral readouts that chronic antidepressant treatment is effective in reversing an anxiety/depression-like phenotype induced by excess glucocorticoids.

The effects of chronic corticosterone treatment on the response of the HPA axis to an acute stress were also looked at. The increase of corticosterone elicited by stress in the control mice was markedly attenuated in corticosterone treated animals (FIG. 9F) [two-way ANOVA with significant effect of pretreatment, treatment factor and pre-treatment×treatment interaction for corticosterone levels (**p<0.01)]. Fluoxetine and imipramine had no effect on stress induced corticosterone levels, both in baseline conditions and after chronic corticosterone treatment.

Chronic Fluoxetine Treatment after Long-Term Corticosterone Exposure Affects all Stages of Adult Hippocampal Neurogenesis.

To investigate the potential cellular mechanisms underlying the behavioral effects of fluoxetine, changes in adult hippocampal neurogenesis, that were hypothesized to be relevant for antidepressant action, were evaluated (Santarelli et al., 2003; Airan et al., 2007).

In agreement with previous observations (Murray et al., 2008; Qiu et al., 2007), chronic corticosterone exposure mimicked the effect of chronic stress on cell proliferation (Surget et al., 2008), decreasing BrdU-positive clusters in the dentate gyrus of the adult mouse hippocampus (−26%) (FIG. 10A) [Two-way ANOVA with significant effect of treatment factor and sampling pre-treatment×treatment interactions (**p<0.01)]. This change in cell proliferation induced by corticosterone was completely reversed by 3-weeks of fluoxetine treatment (18 mg/kg/day). Interestingly, fluoxetine induced an effect on proliferation in corticosterone treated mice but not in non-corticosterone treated animals (BrdUpositive clusters: from 89.5±13.6 in corticosterone treated animals to 120.7±7.3 in corticosterone/fluoxetine group).

Although chronic corticosterone treatment alone altered cell proliferation, it did not affect the survival of newborn neurons (FIG. 10G) or the number of dendrites and dendritic morphology in doublecortin positive cells (FIG. 3H-I). A similar lack of effect on cell survival has been observed after chronic mild stress in rats (Heine et al., 2004; Airan et al., 2007). Furthermore, as previously described, chronic fluoxetine increased the number of doublecortin positive cells with tertiary dendrites and the maturation index in control animals (FIG. 10H, 10I) (Wang et al., 2008). The effect of fluoxetine is even more pronounced in the presence of corticosterone for survival (FIG. 10B, two-way ANOVA with significant effect of treatment factor, **p<0.01) as well as for the number of doublecortin positive cells and their dendritic morphology [FIG. 10H; significant effect of treatment factor and pre-treatment factor, (**p<0.01); [FIG. 3I; two-way ANOVA with significant effect of treatment factor (**p<0.01)]. These results indicate that antidepressants stimulate all stages of adult neurogenesis in an animal with an anxiety/depression-like phenotype.

### The Behavioral Effects of Fluoxetine in the Chronic Corticosterone Model are Mediated by Both Neurogenesis and Neurogenesis-Independent Mechanisms.

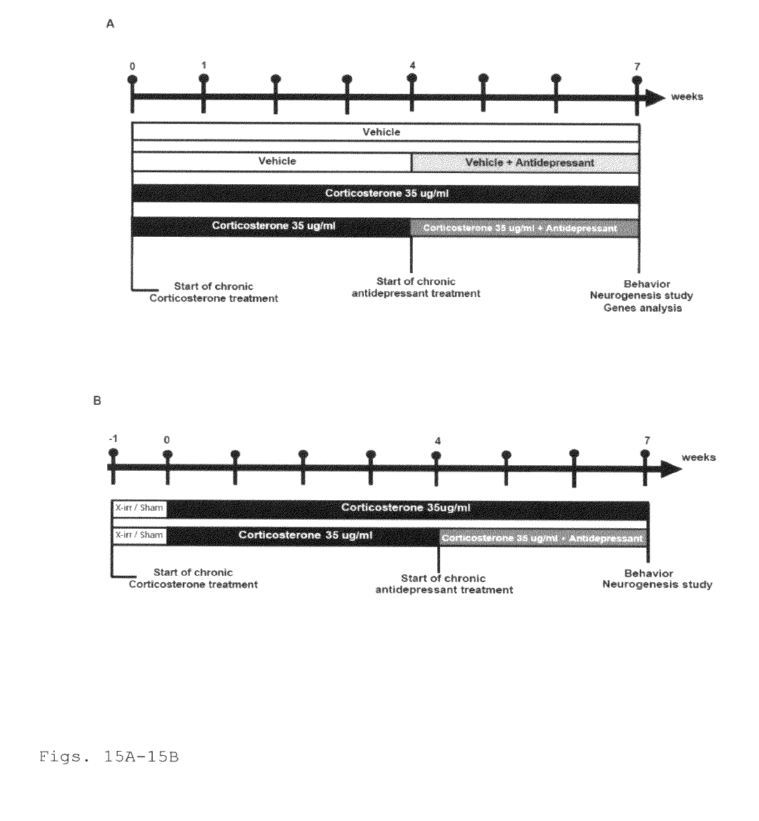

To assess whether adult neurogenesis is required for the antidepressant-mediated reversal of chronic corticosterone treatment in several behavioral tasks, animals were then submitted to focal hippocampal X-irradiation prior to a chronic corticosterone regimen alone or in combination with fluoxetine (see timeline, FIG. 15B).

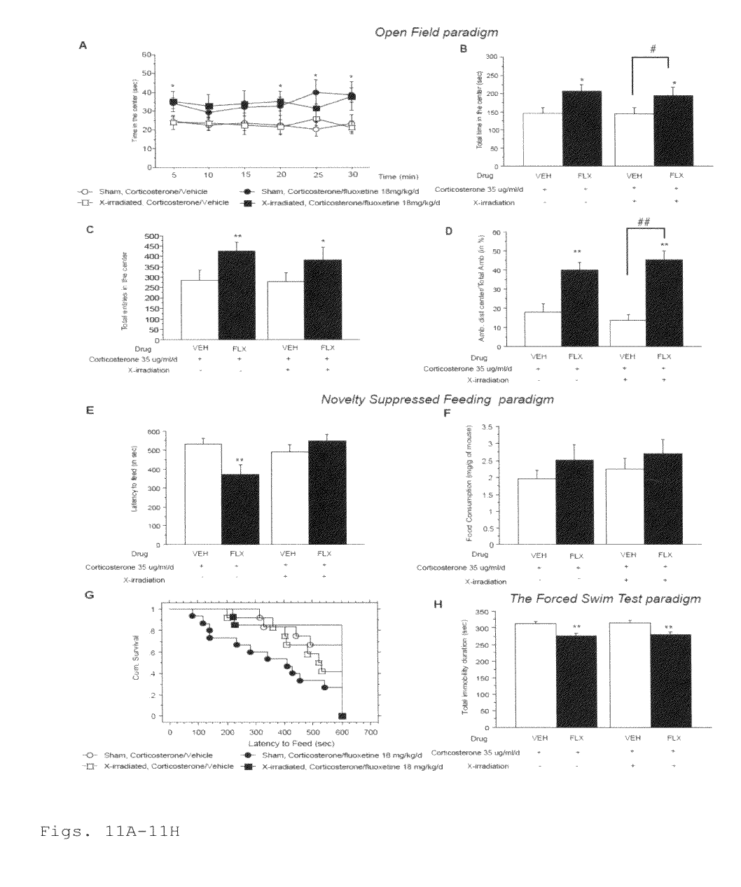

In the Open Field paradigm, the complete loss of hippocampal neurogenesis did not impact the anxiety/depression-like effects of chronic corticosterone. Moreover, the efficacy of fluoxetine was not modified in irradiated mice for all the OF parameters tested (FIGS. 11A, 11B, 11C, 11D). Thus, the total decrease in the time spent in the center (sham, 144.7 s±16.2 and X-ray, 143.2 s±18.4 in corticosterone-treated animals), the total number of entries (sham, 285 s±45.1 and X-ray, 275.2 s±40.1 in corticosterone-treated animals) and the ratio center/total distance traveled (sham, 17.9 s±4.4 and X-ray, 13.2 s±3.2 in corticosterone-treated animals) for all sessions after 7 weeks of corticosterone treatment, were reversed by chronic fluoxetine treatment regardless of whether the mice were exposed to X-irradiation [FIG. 11A, 11B; two-way ANOVA with significant treatment factor (*p<0.05)].

In contrast, the effects of fluoxetine to reverse the anxiety/depressive-like state induced of chronic corticosterone in the NSF paradigm was completely abolished by hippocampal irradiation (from 371.3 s±50.29 in sham corticosterone/fluoxetine group to 546.2 s±36.5 in irradiated corticosterone/fluoxetine group) [FIG. 11E, 11G; two-way ANOVA with significant interaction between irradiation and treatment, **p<0.01], suggesting a dependence on adult hippocampal neurogenesis. Home cage food consumption was not affected by fluoxetine or irradiation (FIG. 11F).

In the mouse FST, the fluoxetine-induced decrease in immobility duration in corticosterone treated animals was not affected by focal irradiation (FIG. 11H).

Taken together, these results demonstrate that hippocampal neurogenesis is required for the behavioral activity of fluoxetine in the NSF test but not in the OF and FST, suggesting distinct underlying mechanisms.

Chronic Fluoxetine Treatment Restored Normal Levels of β-Arrestin 1 and 2, and Giα2 mRNA in the Hypothalamus but not in the Amygdala and the Hippocampus of Corticosterone-Treated Animals.

Next, further exploration of the neurogenesis-independent mechanism responsible for the anxiolytic/antidepressant-like activity of fluoxetine was conducted. To this end, the assessment included whether there were any changes in the expression of candidate genes, previously linked to mood disorders (Avissar et al., 2004; Schreiber and Avissar, 2007; Perlis et al., 2007; de Kloet et al., 2005) in different brain regions.

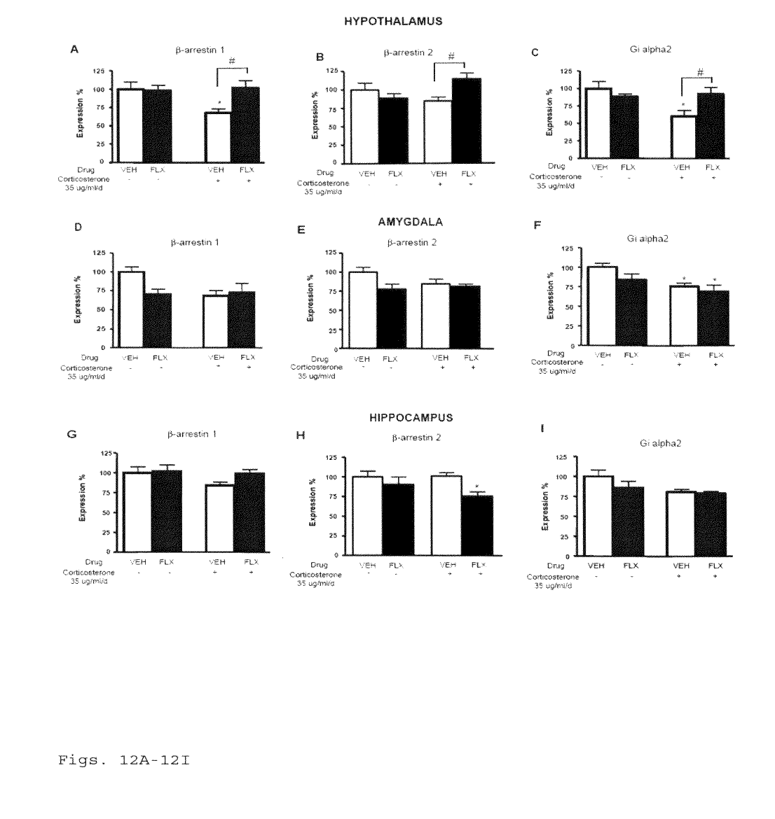

Long-term exposure to corticosterone (35 ug/ml/day) significantly decreased β-arrestin 1 expression in the hypothalamus and there was a similar trend in the amygdala (FIG. 12A, 12D), but did not affect expression in the hippocampus (FIG. 12G) (one-way ANOVA for gene expression in the hypothalamus, **p<0.01). Giα2 expression is also significantly decreased with chronic corticosterone treatment in the hypothalamus and the amygdala (FIG. 12C, 12F) (one-way ANOVA for gene expression in the hypothalamus and the amygdala, **p<0.01. Interestingly, the decrease of β-arrestin 1 (FIG. 12A) and Giα2 (FIG. 12C) gene expression after 7 weeks of corticosterone treatment was totally reversed by chronic fluoxetine treatment only in the hypothalamus but not in the amygdala and the hippocampus (FIG. 12D, 12F, 12G, 121) (one-way ANOVA for gene expression in the hypothalamus, **p<0.01). It was also found β-arrestin 2 expression, a trend of decreased expression (−16%) was that with reversed with fluoxetine treatment in the hypothalamus but not in the amygdala (FIG. 5B, 5E, 5H) (corticosterone/Vehicle group versus corticosterone/fluoxetine group in the hypothalamus, p<0.05). Surprisingly, in the hippocampus, fluoxetine had an opposite effect on β-arrestin 2 levels.

### β-Arrestin 2 is Necessary for the Anxiolytic/Antidepressant Effects of Chronic Fluoxetine.

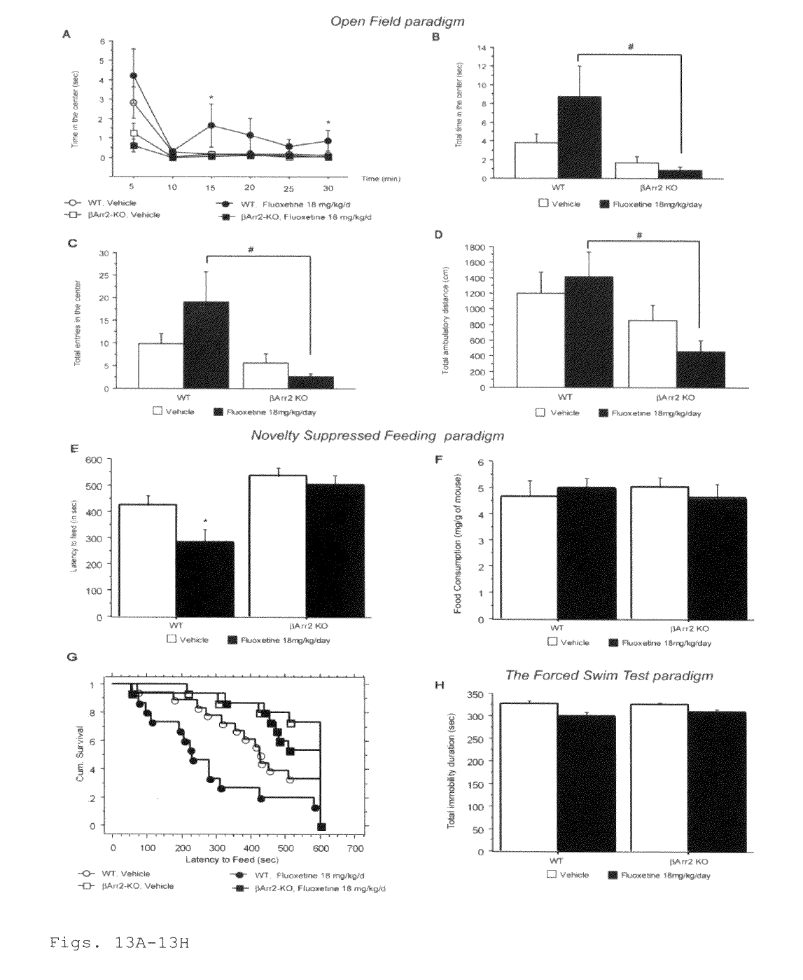

The contribution β-arrestin 2 to the behavioral effects of a 4 week treatment with fluoxetine (18 mg/kg/day) was investigated. In the OF paradigm, β-arrestin 2 knock out mice (S129/Sv×C57BL/6) in the control group display an anxious-like phenotype evidenced by a decreased of the time spent in the center as well as a decreased number of entries in the center relative to the untreated wild-type mice. Chronic fluoxetine treatment had an effect on all anxiety parameters in wild-type animals, resulting in increased time spent in the center (FIG. 13A, 13B) and total number of entries in the center (6C). Interestingly, planned comparisons unveiled that this effect of fluoxetine treatment is abolished in β-arrestin 2 knock out mice [two-way ANOVA, **p<0.01, FIG. 6B, significant effects of pretreatment (**p<0.01)]. This absence of effects of fluoxetine in β-arrestin 2 knock out mice is also observed with the total number of entries in the center and the total ambulatory distance [FIG. 13C, 13D, significant effect of pre-treatment (**p<0.01)].

Next, the effects of fluoxetine in β-arrestin 2 knock out mice was tested using the NSF paradigm. Importantly, untreated β-arrestin 2 knock out display an anxious/depressive phenotype evidenced by an increased latency to feed relative to the untreated wild-type mice. Furthermore, while in wild-type mice fluoxetine significantly decreased the latency to feed in this anxiogenic/depressive setting, fluoxetine had no effect in mutant mice (FIG. 13E,13G: Kaplan-Meier survival analysis, Mantel-Cox log-rank test *p<0.05). Food consumption in the home cage was not altered (FIG. 13F; two-way ANOVA, p>0.4). Taken together, these data indicate that β-arrestin 2 is required for the behavioral effects of fluoxetine in the OF and NSF paradigms.

Lastly, the effects of fluoxetine in β-arrestin 2 knock out mice were tested using the mouse FST. β-arrestin 2 knockout mice treated with fluoxetine were found to behave similarly to wild-type mice in that they displayed a decrease in immobility relative to the control group. Therefore, in contrast to the Open Field and NSF results, β-arrestin 2 is not necessary for the behavioral effects of chronic fluoxetine in the mouse FST [two-way ANOVA, FIG. 6H, significant effects of treatment (p<0.01)].

### Discussion

The data indicate that the behavioral activity of antidepressants such as fluoxetine requires both neurogenesis-dependent and -independent mechanisms. Evidence also demonstrated that some of the effects of fluoxetine are mediated by a β-arrestin signaling pathway.

Elevation of Glucocorticoids Levels Induce an Anxiety/Depressive State in Mice that is Reversed by Chronic Antidepressants.

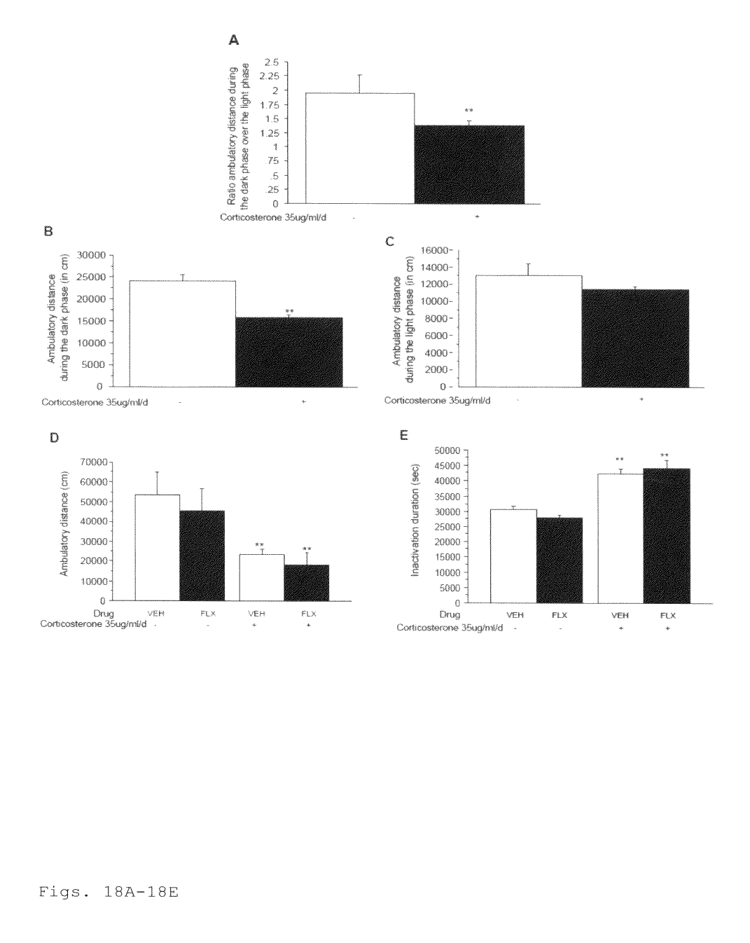

Enhanced activity of the HPA axis involving elevated glucocorticoid levels is considered as a key neurobiological alteration in major depression (for review see Antonijevic et al., 2006). In depressed patients, many studies have shown that successful antidepressant therapies are associated with normalization of impairments in the HPA axis negative feedback (Greden et al., 1983; Linkowski et al 1987; Heuser et al 1996; Holsboer-Trachsler et al., 1991). This elevation of glucocorticoid levels in human has been modeled in rodent to reproduce an anxiety and depressive-like state (Ardayfio and Kim, 2006; Murray et al., 2008; Zhao et al., 2008; Gourley et al., 2008). The model of elevated glucocorticoid herein was able to blunt the response of the HPA axis as shown by the markedly attenuated stress-induced corticosterone levels observed in these mice (FIG. 9F). This is probably a consequence of the negative feedback exerted by corticosterone on the HPA axis. Consistent with previous findings, these results demonstrated that an elevation of glucocorticoid levels is sufficient to induce anxiety in C57BL/6Ntac mice as measured by the decrease in center measures in the OF paradigm as well as with the increase in latency to feed in the NSF (FIG. 8, FIG. 15). A depressive-like state in the same corticosterone-treated animals was also observed as measured by a deterioration of the coat state, a decreased grooming behavior and a flattened circadian rhythm with reduction in home cage activity (FIG. 9, FIG. 18). These symptoms are similar to those elicited by chronic stress (Surget et al., 2008). Similarly, a subset of depressed patients with elevated cortisol has been shown to display anhedonia, cognitive dysfunctions/distortions and personal neglect (Morgan et al., 2005). Therefore, chronic corticosterone treatment appears to model an anxious and depressed-like state in mice.

In a marked contrast to the OF and NSF paradigms, the FST was the only behavioral model in which antidepressants exerted effects in normal “nonanxious/depressed” mice. The absence of antidepressant effect in both NSF and OF paradigms when normal “non-depressed” mice were used, suggests that different neurobiological mechanisms are recruited by antidepressants when animals are examined in baseline rather than in pathological conditions. Interestingly, when a more anxious strain is used such as the 129SvEv mice, it is possible to detect effects of chronic antidepressants in baseline conditions (Santarelli et al., 2003). It is noteworthy that neither fluoxetine nor imipramine restored normal levels of corticosterone after an acute stressor, which suggests that their mechanism of action may be independent of the HPA axis.

### Enhanced Effects of Fluoxetine Treatment on Neurogenesis in Corticosterone-Treated Mice.

Glucocorticoids and antidepressants have been shown to modulate adult neurogenesis in opposite directions and hippocampal neurogenesis is required for some of the effects of antidepressants (Gould et al., 1992; McEwen, 1999; Duman et al., 2000; Malberg et al., 2000; McEwen, 2001; Santarelli et al., 2003; Airan et al., 2007; Surget et al., 2008; Murray et al., 2008; Qui et al., 2007, Conrad et al., 2007). Since it was previously demonstrated that antidepressants increase all stages of neurogenesis including proliferation, maturation and survival in normal mice, understanding was sought of the effects of fluoxetine on neurogenesis in mice that were in an anxious and depressed-like state was.

In agreement with previous findings (Murray et al., 2008; Qui et al., 2007), a reduction in the proliferation of progenitor cells after chronic corticosterone treatment was observed (FIG. 10), demonstrating a role for glucocorticoids in the regulation of the proliferation stage of the neurogenic process. Indeed, it had been reported that ablation of the adrenal glands abolishes stress-induced decreases of cell proliferation (Tanapat et al., 2001). Interestingly, the effects of corticosterone on neurogenesis are limited to the proliferation stage and not the survival or maturation of newborn neurons. Similar results were observed in rat (Heine et al., 2004) and it has been proposed that a decrease in apoptosis counteracts the reduction in neurogenesis elicited by stress and explains the absence of change in number of newborn neurons after chronic stress.

Surprisingly, chronic fluoxetine treatment did not affect hippocampal cell proliferation in non-corticosterone treated C57BL/6Ntac mice. Strain differences in hippocampal adult proliferation have been reported (Schauwecker, 2006, Navailles et al., 2008) and C57BL/6 strain exhibit one of the highest numbers of proliferating cells within the subgranular zone, as compared to other strain office.

Interestingly, the effects of fluoxetine on all stages of neurogenesis (proliferation, differentiation and survival) were more pronounced in corticosterone treated mice than in controls. These enhanced effects may be due to change in the serotonin system elicited by chronic stress. In fact, it has been shown that chronic stress results in a desensitization of 5-HT1A autoreceptors (Hensler et al., 2007; and data not shown) which is likely to result in an increase in serotonin release and therefore possibly in a stronger effect of fluoxetine. There is also an interesting parallel between these enhanced effects of fluoxetine on neurogenesis and the fact that fluoxetine is more active behaviorally in the corticosterone-treated mice.

### Neurogenesis-Dependent and -Independent Mechanisms.

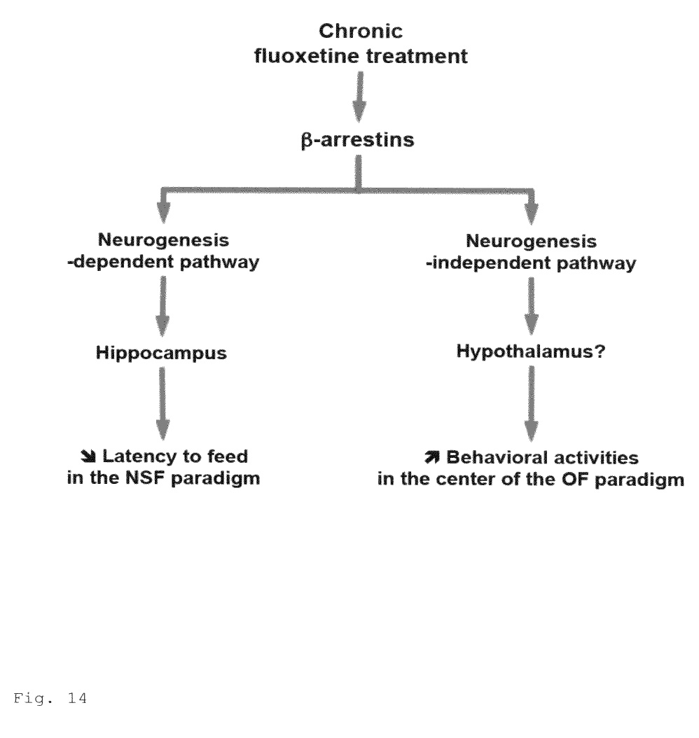

Earlier studies have shown that some of the effects of antidepressants in the NSF test require hippocampal neurogenesis (Santarelli et al., 2003). Therefore, it was hypothesized that the effect of fluoxetine on the anxiogenic/depressive-like state in corticosterone-treated mice may also require neurogenesis. Indeed, in the corticosterone model, the effects of fluoxetine in the NSF test were blocked by X-irradiation. However, in the same animals, in the OF and the FST, ablation of hippocampal neurogenesis did not modify the anxiolytic/antidepressant-like activity of fluoxetine (FIG. 5). These behavioral effects are therefore likely to recruit different pathways (FIG. 14). To date, this is the first study, using a model of anxiety/depression in mice, showing that neurogenesis dependent and independent mechanisms are both necessary for the effects of fluoxetine. Overall, these studies suggest that the hippocampal neurogenesis plays an important role in the behavioral effects of fluoxetine. However, there is accumulating evidence that other brain regions are also involved in antidepressant-like activity including amygdala, nucleus accumbens or cingulate cortex.

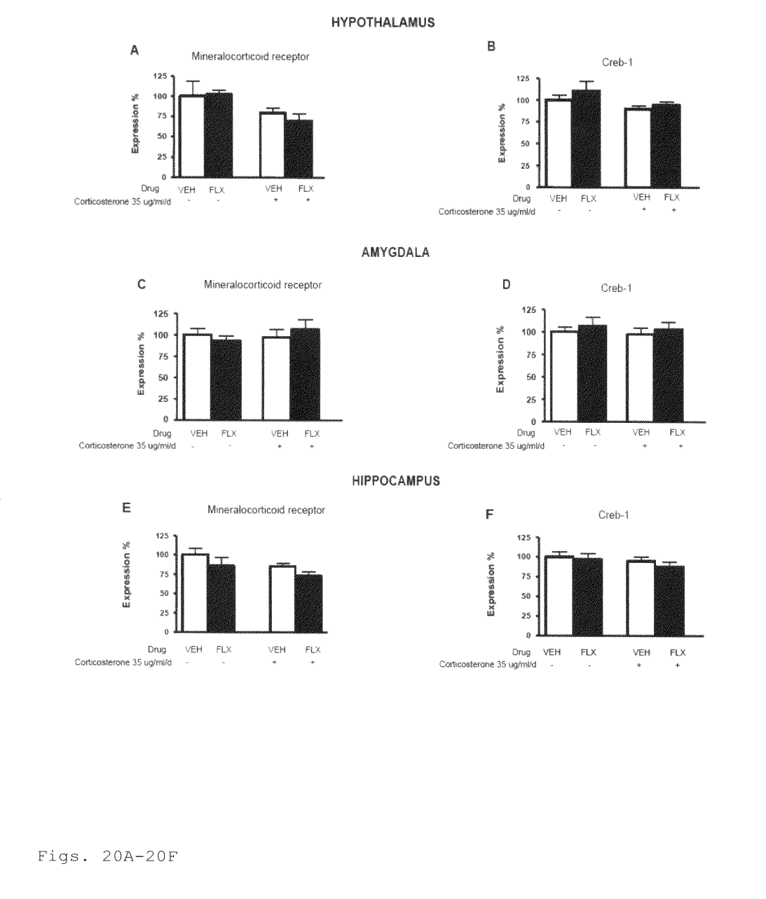

To explore the mechanism underlying the neurogenesis-independent effects of fluoxetine, gene expression profiles in the hypothalamus amygdala and hippocampus, three brains structures involved in the stress response were analyzed (Nemeroff and Owens, 2004; McEwen et al., 2004; Mayberg et al., 2005; Joels, 2008). The variations in mRNA levels encoding candidate genes selected for their implication in mood disorders including G protein-coupled receptors (GPCR), transcription factors and genes involved in the stress response were examined (Koch et al., 2002; Calfa et al., 2003; Avissar et al., 2004; de Kloet et al., 2005; Matuzany-Ruban et al., 2005; Schreiber and Avissar, 2007; Perlis et al., 2007; Holsboer, 2008; Avissar et al., 1998). Among these genes, only 3 displayed a change in mRNA levels in the chronic corticosterone group that was reversed by fluoxetine treatment. Furthermore this bidirectional change was only observed in the hypothalamus. Interestingly all 3 genes are involved in GPCR 2; FIG. 12 and FIG. 20). The present data are consistent with previous findings in animal and human studies showing decreases in β-arrestin 1 and 2 or Giα2 in depression or after stress and reversal of these changes by various antidepressant treatment (Avissar et al., 1998; Dwivedi et al., 2002; Avissar et al., 2004). Interestingly, corticotropin-releasing factor type 1 (CRF(1)) receptor, a potential target for the treatment of depression/anxiety and other stress-related disorders, has been shown to recruit β-arrestin 2 (Oakley et al., 2007). Moreover, Beaulieu and colleagues (2008) have recently shown that lithium, a drug used in the management of mood disorders, exerts some of its biochemical and behavioral effects via a β-arrestin signaling complex.

### β-Arrestin 2 is Required for Both Neurogenesis-Dependent and Independent Effects of Fluoxetine.

Interestingly, the effects of chronic corticosterone on behavior were similar to those of the β-arrestin 2 ablation. Given that chronic corticosterone treatment decreases β-arrestin levels (particularly in the hypothalamus), it is possible that β-arrestin 2 (FIG. 12), at least in part, is responsible for mediating the effects of corticosterone on behavior. Furthermore, β-arrestin 2 knockout mice displayed a reduced response to fluoxetine in the Open Field and Novelty Suppressed Feeding paradigms. This suggests that β-arrestin 2 modulates the behavioral response to fluoxetine in both neurogenesis-independent and dependent tasks. To further understand how β-arrestin may regulate multiple effects of chronic corticosterone and fluoxetine treatments on behavior, future work will require the usage of tissue-specific knockouts. Classical β-arrestin functions include desensitization of G-protein coupled receptors (Gainetdinov et al., 2004), so it is possible that β-arrestin 2 may be important for desensitization of 5-HT1A receptors in the Raphe Nucleus, a process that has been hypothesized as necessary for the effects of fluoxetine (Artigas et al., 1996). However, the results suggest that 5-HT1A autoreceptor desensitization in response to chronic fluoxetine is normal in β-arrestin 2 knockout mice. Alternatively, other cell signaling functions of β-arrestins have also been uncovered (Pierce and Lefkowitz, 2001, Beaulieu et al., 2005, Lefkowitz et al., 2006; Beaulieu et al., 2008) and some of lithium's behavioral effects appear to be mediated by a β-arrestin 2/Akt/Gsk3β signaling pathway. Therefore, it is possible that β-arrestin 2 serves also as a major signaling intermediate for the antidepressant effects of fluoxetine (FIG. 14).

An anxiety/depression-like model based on elevation of glucocorticoid levels that offers an easy and reliable alternative to existing models such as the various chronic stress paradigms has been developed and disclosed herein. It is also the first model that allows the simultaneous study of multiple effects of antidepressant treatment in the same animal, some of which are neurogenesis-dependent while others are not.

### Experimental Procedures

### Subjects

Adult male C57BL/6Ntac mice were purchased from Taconic Farms (Germantown, N.Y., USA; Lille Skensved, Denmark). Male heterozygous β-arrestin 2+/− and heterozygous female mutant β-arrestin+/− mice (age 4-6 months, 25-30 g body weight) were bred on a mixed S129/Sv×C57BL/6 genetic background raised at the animal facility of Columbia University (New York, USA). Resulting pups were genotyped by polymerase chain reaction as described previously (Beaulieu et al., 2008). All corticosterone treated mice were 7-8 weeks old and weighed 23-35 g at the beginning of the treatment, and were maintained on a 12 L:12 D schedule (lights on at 0600) and housed in groups of five of the same strain. β-arrestin 2 mice began receiving fluoxetine at 3 months. Food and water were provided ad libitum. Behavioral testing occurred during the light phase between 0700 and 1900 for the OF, NSF and FST, splash test. All testing was conducted in compliance with the NIH laboratory animal care guidelines and with protocols approved by the Institutional Animal Care and Use Committee (Council directive #87-848, Oct. 19, 1987, Ministère de l'Agriculture et de la Forêt, Service Vétérinaire de la Santé et de la Protection Animale, permissions #92-256 to D.J.D.).

### Drugs

Corticosterone (4-pregnen-11b-DIOL-3 20-DIONE 21-hemisuccinate from Sigma, St Louis, Mo.) was dissolved in vehicle (0.45% hydroxypropyl-β-cyclodextrin (β-CD), Sigma, St Louis, Mo.). Imipramine hydrochloride (40 mg/kg per day in the drinking water) and fluoxetine hydrochloride (18 mg/kg per day in the drinking water) were purchased from Sigma (St Louis, Mo., USA) and Anawa Trading (Zurich, Switzerland) respectively. Corticosterone (7 ug/ml or 35 ug/ml per day, equivalent to 1 and 5 m/kg/day) was delivered alone or in presence of antidepressant in opaque bottles to protect them from light, available ad libitum in the drinking water and replaced twice a week. CORT treatment did not modify antidepressant brain exposure (data not shown). For all the studies, control mice received β-CD. For β-arrestin 2 knock out mice, fluoxetine was delivered by a standard gavage protocol (18 mg/kg/day).

### Behavioral Testing

The originality of the protocol described here included that the same cohort of animal was tested in three different behavioral models of anxiety and depression. So each animal, over one week, was successively tested in the OF, the NSF and the mouse FST.

### Open Field Paradigm

The procedure described previously was used (Dulawa et al., 2004). Motor activity was quantified in four Plexiglas open field boxes 43 times 43 cm2 (MED Associates, Georgia, Vt.). Two sets of 16 pulse-modulated infrared photobeams were placed on opposite walls 2.5-cm apart to record x-y ambulatory movements. Activity chambers were computer interfaced for data sampling at 100-ms resolution. The computer defined grid lines that divided each Open Field into center and surround regions, with each of four lines being 11 cm from each wall. Dependent measures in the center were the total time and the number of entries over a 30-min period of test. The whole session was also divided in 5 periods to analyze the time spent in the center every 5 min. The overall motor activity was quantified as the total distance traveled (cm) or distance traveled in the center divided by total distance traveled.

### Novelty Suppressed Feeding Paradigm

The novelty suppressed feeding paradigm (NSF) is a conflict test that elicits competing motivations: the drive to eat and the fear of venturing into the center of brightly lit arena. Latency to begin eating is used as an index of anxiety-like behavior, because classical anxiolytic drugs decrease this measure. The NSF test was carried out during a 10-min period as previously described (Santarelli et al., 2003; David et al., 2007). Briefly, the testing apparatus consisted of a plastic box (50×50×20 cm), the floor of which was covered with approximately 2 cm of wooden bedding. Twenty-four hours prior to behavioral testing, all food was removed from the home cage. At the time of testing, a single pellet of food (regular chow) was placed on a white paper platform positioned in the center of the box. An animal was placed in a corner of the box, and a stopwatch was immediately started. The latency to eat (defined as the mouse sitting on its haunches and biting the pellet with the use of forepaws) was timed. Immediately afterwards, the animal was transferred to its home cage, and the amount of food consumed by the mouse in the subsequent 5 min was measured, serving as a control for change in appetite as a possible confounding factor. Each mouse was weighed before food deprivation and before testing to assess the percentage of body weight loss (data not shown).

### Forced Swim Test

The forced swim test procedure was modified relative to the traditional method, so as to enhance sensitivity for detecting the putative antidepressant activity of drugs (Porsolt et al., 1977). The modifications consist of an increase in water depth (Dulawa et al., 2004). Mice were placed into plastic buckets (19 cm diameter, 23 cm deep, filled with 23-25° C. water) and videotaped for 6 min to score immobility duration.

### Changes in Coat State

The state of the coat was assessed at the end of the corticosterone regimen (end of seventh weeks) in the presence or absence of 3-weeks of fluoxetine treatment. The total score resulted from the sum of the score of five different body parts: head, neck, dorsal/ventral coat, tail, fore-/hindpaws. For each of the five body areas, a score of 0 was given for a well-groomed coat and 1 for an unkempt coat (Griebel et al., 2002; Santarelli et al., 2003).

### Splash Test

The grooming latency was assessed at the end of the corticosterone regimen (end of seventh week) in the presence or absence of 3-weeks of fluoxetine treatment. This test consisted in squirting 200 ul of a 10% sucrose solution on the mouse's snout. The grooming duration and grooming frequency were then recorded.

### Stress Evoked Increase of Corticosterone Levels

Adult male C57BL/6Ntac mice were exposed to a 6 minutes swim stress. Mice were placed into plastic buckets (19 cm diameter, 23 cm deep, filled with 23-25° C. water) and sacrificed 12 min after the end of the test. Blood was collected into ice-chilled tubes containing EDTA and centrifuged at 3000 rpm for 10 min (at 4° C.) for separation of plasma, and plasma samples were stored at −80° C. until assayed. Plasma corticosterone levels were determined with a commercially available RIA kit (Rat Corticosterone RIA, DSL-80100; Diagnostic Systems Laboratories, Inc. Webster, Tex.; sensitivity limit: 20 ng/ml). ACTH was measured directly in plasma using an ImmuChem™ Double Antibody hACTH 1251 RIA kit (No. 07-106101; MP Biomedicals, LLC, Orangeburg, N.Y.) with a sensitivity limit ˜5.7 pg/ml. All samples were measured simultaneously to reduce inter-assay variability.

### X-Ray Irradiation

Mice were anesthetized with ketamine and xylazine (100 mg/ml ketamine; 20 mg/ml xylazine), placed in a stereotaxic frame and exposed to cranial irradiation using a Siemens Stabilopan X-ray system operated at 300 kVp and 20 mA. Animals were protected with a lead shield that covered the entire body, but left unshielded a 3.22×11-mm treatment field above the hippocampus (interaural 3.00 to 0.00) exposed to X-Ray. Dosimetry was done using a Capintec Model PR06G electrometer ionization chamber and Kodak Readypack Radiographic XV films. The corrected dose rate was approximately 1.8 Gy per min at a source to skin distance of 30 cm. The procedure lasted 2 min and 47 sec, delivering a total of 5 Gy. Three 5 Gy doses were delivered on days 1, 4 and 8.

### Immunohistochemistry

### BrdU Labeling for Proliferation and Survival Study

The effects of a chronic corticosterone treatment in presence or absence of fluoxetine treatment were assessed on cell proliferation or cell survival. Mice were administered with BrdU (150 mg/kg, i.p. dissolved in saline), 2 h before sacrifice or twice a day during days before the start of the corticosterone treatment for cell proliferation and cell survival respectively. After anesthesia with ketamine (100 mg/kg), mice were perfused transcardially (cold saline for 2 min, followed by 4% cold paraformaldehyde at 4° C.). The brains were then removed and cryoprotected in 30% sucrose and stored at 4° C. Serial sections (35 μM) were cut through the entire hippocampus (plate 41-61; Franklin and Paxinos, 1997) on a cryostat and stored in PBS with 0.1% NaN₃. For DAB staining, sections were mounted on slides and boiled in citric acid (pH 6.0) for 5 min, rinsed with PBS, and treated with 0.01% trypsin in Tris/CaCl₂ for 10 min. Brain sections were incubated for 30 min with 2N HCl and blocked with 5% NGS. Sections were then incubated overnight at room temperature with anti-mouse BrdU (1:100). After washing with PBS, sections were incubated for 1 hr with secondary antibody (1:200 biotinylated goat anti-mouse) followed by amplification with an avidinbiotin complex. The staining was visualized with DAB. For the quantification of BrdU labeling, a stereological procedure was used as previously described (Malberg et al., 2000).

### Doublecortin (DCX) Labeling for Maturation Index Study

For doublecortin staining, the procedure consisted of the following steps (Wang et al., 2008): sections were rinsed in PBS, treated with 1% H₂O₂ in 1:1 PBS and methanol for 15 min to quench endogenous peroxidase activity (and to enhance dendritic staining), incubated in 10% normal donkey serum and 0.3% Triton X-100 for 30 min, and then incubated overnight at 4° C. in primary antibody for doublecortin (goat; 1:500; Santa Cruz Biotechnology, Santa Cruz, Calif.). The secondary antibody was biotinylated donkey anti-goat (1:500) (Jackson ImmunoResearch, West Grove, Pa.) in PBS for 2 hr at room temperature. Sections were developed using avidin-biotin complex (Vector, USA) and DAB kit. Bright-field images were taken with a Zeiss (Oberkochen, Germany) Axioplan-2 upright microscope. Stereological procedure was used to quantify labeled cells (Wang et al., 2008). DCX+ cells were subcategorized according to their dendritic morphology: DCX+ cells with no tertiary dendritic processes and DCX+ cells with complex, tertiary dendrites. The maturation index was defined as the ratio of DCX+ cells possessing tertiary dendrites over the total DCX+ cells.

### Transcription Analysis

Tissue preparation: Animals were sacrificed by cervical dislocation. Selected brain regions were dissected and placed in tubes containing RNAlater (Ambion), incubated at 4 degrees C. overnight and stored at −80 degrees C. until processing.

RNA Extraction and cDNA Preparation

Brain regions (10-20 mg) were homogenized for 20 sec at medium speed in 1.25 ml lysis/denaturation buffer (Ambion) using an Autogizer™ (Tomtec). Total RNA was isolated from 100-300 ul aliquots of the homogenate using the RNAqueous™ 96 automated kit (Ambion) according to the manufacturer's protocol. A second DNase I digestion was incorporated after elution of the RNA from the Ambion filter plate to remove residual genomic DNA. Digestion was performed for 1 hr at room temperature using DNase I™ (Invitrogen) and the buffer supplied with the enzyme. After inactivation of the DNase with EDTA and heat, the RNA was desalted with a Multiscreen™ filter plate (Millipore) and stored at −80° C. Conversion of total RNA into first strand cDNA was accomplished with Superscript II™ enzyme (Invitrogen) followed by desalting over a Multiscreen plate. Approximately 1 ug of total RNA was used for each cDNA reaction. The yield of cDNA was determined using Quant-iT Oligreen™ reagent (Invitrogen). Prior to the Oligreen assay, total RNA carried over from the cDNA reaction was hydrolyzed with NaOH and heat, followed by neutralization with Tris buffer. This treatment eliminates any contribution of the RNA to the Oligreen signal. The unknown cDNA samples were compared to a standard curve derived using a 18mer oligonucleotide. Replica cDNA plates containing 3 ng of cDNA per well were prepared using an Evolution P3™ workstation (PerkinElmer). Each animal in a given experiment was represented by one well on each plate and each plate always contained the control and treatment groups.

qPCR Analysis

Quantitative PCR (qPCR) was carried out in 25 ul reactions using Full Velocity™ enzyme (Stratagene). Plates were run on either a Stratagene MX3000P™ or an Applied Biosystems 7900 HT instrument. The cycling parameters were set based on recommendations from the enzyme manufacturer. One gene expression profile was analyzed per PCR plate and duplicate plates were run for each gene. Two housekeeping genes, cyclophilin and GAPDH, were included in the gene list and were used to normalize the expression results obtained from the other genes of interest (see data analysis section). The sequences of the primers and probes for each gene are listed in supplemental table 1. Duplicate cycle thresholds (Ct values) were obtained for each gene/region and averaged. The values for cyclophilin and GAPDH were combined and used to normalize the expression values from the other genes by employing the delta Ct method. After converting delta Ct values to percentage, the mean and SEM of each animal group (controls and experimental) was calculated.

### Data Analysis and Statistics

Results from data analyses were expressed as mean±SEM. Data were analyzed using StatView 5.0 software (SAS Institute, Cary, N.C.) or GraphPad Prism. For all experiments one-way, two-way or three way ANOVA with repeated measure were applied to the data as appropriate. Significant main effects and/or interactions were resolved followed by Fisher's protected least significant difference (PLSD) post hoc ANOVAs analysis or post hoc unpaired t tests or Newman-Keuls test as appropriate. In the NSF test, the Kaplan-Meier survival analysis was also used because of the lack of normal distribution of the data. Animals that did not eat during the 10 min testing period were censored. Mantel-Cox log-rank test was used to evaluate differences between experimental groups.

### Tables

TABLE 1
Oligonucleotide sequences (5′→ 3′) used
for the qPCR analysis (from top to bottom
SEQ ID NOs. 1-21, respectively)

| Gene | | oligo sequence (5′-3′) |
|---|---|---|
| ARRB1 | F | CCACCAGACAGTTCCTCATGTC |
| ARRB1 | R | CATTGACGCTGATGGGTTCTC |
| ARRB1 | T | CCCTGCACCTTGAGGCATCTCTGGATA |
| ARRB2 | F | TCCGCTATGGCCGAGAAG |
| ARRB2 | R | CCTGGTAGGTGGCGATGAAC |
| ARRB2 | T | ATGTACTGGGCCTGTCTTTCCGCAAA |
| CREB | F | TCAAGCTGCCTCTGGTGATG |
| CREB | R | GGAGGACGCCATAACAACTCC |
| CREB | T | AAACATACCAGATTCGCACAGCACCCA |
| Cyclo | F | TTTCGCCGCTTGCTGC |
| Cyclo | R | CTCGTCATCGGCCGTGA |
| Cyclo | T | CATGGTCAACCCCACCGTGTTCTTC |
| GAPDH | F | CAAATTCAACGGCACAGTCAAG |
| GAPDH | R | ACCCCATTTGATGTTAGTGG |
| GAPDH | T | TCATCAACGGGAAGCCCATCACCATCT |
| Gi2 | F | ACCATGGTGTGCAAGCCTG |
| Gi2 | R | GGTAGTAAGCGGCTGAGTCATTG |
| Gi2 | T | TTGGCCGCTCACGGGAATATCAA |
| MR | F | TGTCCTCCTCCACAGCTAGCTT |
| MR | R | GCATGTCAGTGAGGTTCCTTGA |
| MR | T | CAGTTTCCCAGTGCACAGTCCCATCA |

F = forward primer
R = reverse primer
T = TaqMan Probe

TABLE 2
Statistical Summary

| Behavioral | | Statistical | | | Degrees of | | |
|---|---|---|---|---|---|---|---|
| paradigm | Measurement | Test | Comparison | Statistics | freedom | p | Fig. |
| Open Field | Time in the | 3-way repeated | Factor 1- Pre- | F = 24.79 | 1, 390 | <0.01** | 1A |
| Paradigm | center | measures | treatment | | | | |
| | | ANOVA | Factor 2 | F = 3.82 | 2, 390 | <0.01** | |
| | | | Treatment | | | | |
| | | | Factor 3 | F = 4.52 | 5, 390 | <0.01** | |
| | | | Time | | | | |
| | | | Interaction | F = 1.35 | 10, 390 | 0.2 | |
| | | | (F1 × F2 × F3) | | | | |
| | | PLSD | CORT vs Veh | | | <0.01## | |
| | | Post-hoc test | CORT vs CORT/Flx | | | <0.01## | |
| | | | CORT vs CORT/Imi | | | <0.01## | |
| | Total Time in | 2-way ANOVA | Factor 1- Pre- | F = 24.75 | 1, 78 | <0.01** | 1B |
| | the center | | treatment | | | | |
| | | | Factor 2 | F = 3.82 | 2, 78 | <0.01** | |
| | | | Treatment | | | | |
| | | | Interaction | F = 8.32 | 2, 78 | <0.01** | |
| | | | (F1 × F2) | | | | |
| | | PLSD | CORT vs Veh | | | <0.01* | |
| | | Post-hoc test | CORT vs CORT/Flx | | | <0.01## | |
| | | | CORT vs CORT/Imi | | | <0.01## | |
| | Entries in the | 2-way ANOVA | Factor 1- Pre- | F = 20.97 | 1, 78 | <0.01** | 1C |
| | enter | | treatment | | | | |
| | | | Factor 2 | F = 4.81 | 2, 78 | <0.01** | |
| | | | Treatment | | | | |
| | | | Interaction | F = 11.75 | 2, 78 | <0.01** | |
| | | | (F1 × F2) | | | | |
| | | PLSD | CORT vs Veh | | | <0.01** | |
| | | Post-hoc test | CORT vs CORT/Flx | | | <0.01## | |
| | | | CORT vs CORT/Imi | | | <0.01## | |
| | Ambulatory | 2-way ANOVA | Factor 1- Pre- | F = 2.11 | 1, 78 | <0.08 | 1D |
| | Distance | | treatment | | | | |
| | | | Factor 2 | F = 0.413 | 2, 78 | <0.062 | |
| | | | Treatment | | | | |
| | | | Interaction | F = 4.49 | 2, 78 | <0.01** | |
| | | | (F1 × F2) | | | | |
| | | PLSD | CORT vs CORT/Flx | | | <0.01# | |
| | | Post-hoc test | | | | | |
| Novelty | Latency to | 2-way ANOVA | Factor 1- Pre- | F = 11.2 | 1, 80 | <0.01** | 1E |
| Suppressed | feed | | treatment | | | | |
| Feeding | | | Factor 2 | F = 1.48 | 2, 80 | <0.23 | |
| | | | Treatment | | | | |
| | | | Interaction | F = 1.80 | 2, 80 | <0.15 | |
| | | | (F1 × F2) | | | | |
| | | PLSD | CORT vs Veh | | | <0.01** | |
| | | Post-hoc test | CORT vs CORT/Flx | | | <0.01## | |
| | | Kaplan-Meier | | | | <0.01** | 1G |
| | | survival analysis | | | | | |
| | Food | 2-way ANOVA | Factor 1- Pre- | F = 2.1 | 1, 80 | <0.14 | 1F |
| | consumption | | treatment | | | | |
| | | | Factor 2 | F = 1.16 | 2, 80 | <0.30 | |
| | | | Treatment | | | | |
| | | | Interaction | F = 0.65 | 2, 80 | <0.52 | |
| | | | (F1 × F2) | | | | |
| The Forced | Immobility | 2-way ANOVA | Factor 1- Pre- | F = 0.43 | 1, 75 | <0.51 | 1H |
| Swim test | duration | | treatment | | | | |
| | | | Factor 2 | F = 14.8 | 2, 75 | <0.01** | |
| | | | Treatment | | | | |
| | | | Interaction | F = 0.4 | 2, 75 | <0.66 | |
| | | | (F1 × F2) | | | | |
| | | PLSD | CORT vs CORT/Flx | | | <0.01** | |
| | | Post-hoc test | CORT vs CORT/Imi | | | <0.01** | |
| Coat State | | 2-way ANOVA | Factor 1- Pre- | F = 877.23 | 1, 42 | <0.01** | 2C |
| | | | treatment | | | | |
| | | | Factor 2 | F = 7.49 | 1, 42 | <0.01** | |
| | | | Treatment | | | | |
| | | | Interaction | F = 13.93 | 1, 42 | <0.01** | |
| | | | (F1 × F2) | | | | |
| | | PLSD | CORT vs Veh | | | <0.01** | |
| | | Post-hoc test | CORT/Flx vs Veh | | | <0.01** | |
| | | | CORT vs CORT/Flx | | | <0.01## | |
| Splash State | Grooming | 2-way ANOVA | Factor 1- Pre- | F = 0.19 | 1, 42 | <0.66 | 2D |
| | | | treatment | | | | |
| | | | Factor 2 | F = 17.48 | 1, 42 | <0.01** | |
| | | | Treatment | | | | |
| | | | Interaction | F = 8.60 | 1, 42 | <0.01** | |
| | | | (F1 × F2) | | | | |
| | | PLSD | CORT vs Veh | | | <0.01** | |
| | | Post-hoc test | CORT vs CORT/Flx | | | <0.01## | |
| | Frequency of | 2-way ANOVA | Factor 1- Pre- | F = 12.13 | 1, 42 | <0.01** | 2E |
| | grooming | | treatment | | | | |
| | | | Factor 2 | F = 3.05 | 1, 42 | <0.05* | |
| | | | Treatment | | | | |
| | | | Interaction | F = 1.59 | 1, 42 | <0.21 | |
| | | | (F1 × F2) | | | | |
| | | PLSD | CORT vs Veh | | | <0.01** | |
| | | Post-hoc test | CORT vs CORT/Flx | | | <0.01## | |
| Stress evoked | | 2-way ANOVA | Factor 1- Pre- | F = 320.43 | 1, 38 | <0.01** | 2F |
| increase of | | | treatment | | | | |
| corticosterone | | | Factor 2 | F = 4.97 | 2, 38 | <0.01** | |
| levels | | | Treatment | | | | |
| | | | Interaction | F = 4.68 | 2, 38 | <0.01** | |
| | | | (F1 × F2) | | | | |
| | | PLSD | CORT vs Veh | | | <0.01** | |
| | | Post-hoc test | CORT/Flx vs Veh | | | <0.01** | |
| | | | CORT/Imi vs Veh | | | <0.01** | |
| Neurogenesis | Proliferation | 2-way ANOVA | Factor 1- Pre- | F = 2.43 | 1, 12 | <0.14 | 3A |
| | | | treatment | | | | |
| | | | Factor 2 | F = 11.81 | 1, 12 | <0.01** | |
| | | | Treatment | | | | |
| | | | Interaction | F = 17.61 | 1, 12 | <0.01** | |
| | | | (F1 × F2) | | | | |
| | | PLSD | CORT vs Veh | | | <0.05* | |
| | | Post-hoc test | CORT/Flx vs Veh | | | <0.01** | |
| | | | CORT/Flx vs CORT | | | <0.01## | |
| | Survival | 2-way ANOVA | Factor 1- Pre- | F = 0.0007 | 1, 20 | <0.99 | 3B |
| | | | treatment | | | | |
| | | | Factor 2 | F = 4.34 | 1, 20 | <0.05* | |
| | | | Treatment | | | | |
| | | | Interaction | F = 0.487 | 1, 20 | <0.49 | |
| | | | (F1 × F2) | | | | |
| | | PLSD | CORT/Flx vs Veh | | | <0.05* | |
| | | Post-hoc test | CORT/Veh vs | | | <0.05# | |
| | | | CORT/Flx | | | | |
| | Total | 2-way ANOVA | Factor 1- Pre- | F = 3.12 | 1, 16 | <0.09 | 3G |
| | doublecortine | | treatment | | | | |
| | positive cells | | Factor 2 | F = 19.53 | 1, 16 | <0.01** | |
| | | | Treatment | | | | |
| | | | Interaction | F = 3.20 | 1, 16 | <0.09 | |
| | | | (F1 × F2) | | | | |
| | | PLSD | CORT/Flx vs Veh | | | <0.01** | |
| | | Post-hoc test | CORT/Veh vs | | | <0.01## | |
| | | | CORT/Flx | | | | |
| | Doublecortine | 2-way ANOVA | Factor 1- Pre- | F = 3.85 | 1, 16 | <0.06 | 3H |
| | positive cells | | treatment | | | | |
| | W tertiary | | Factor 2 | F = 23.05 | 1, 16 | <0.01** | |
| | dendrites | | Treatment | | | | |
| | | | Interaction | F = 3.11 | 1, 16 | <0.09 | |
| | | | (F1 × F2) | | | | |
| | | PLSD | Flx vs Veh | | | <0.05* | |
| | | Post-hoc test | CORT/Flx vs Veh | | | <0.01** | |
| | | | CORT/Veh vs | | | <0.01## | |
| | | | CORT/Flx | | | | |
| | Maturation | 2-way ANOVA | Factor 1- Pre- | F = 3.62 | 1, 16 | <0.01** | 31 |
| | index | | treatment | | | | |
| | | | Factor 2 | F = 22.76 | 1, 16 | <0.77 | |
| | | | Treatment | | | | |
| | | | Interaction | F = 2.06 | 1, 16 | <0.17 | |
| | | | (F1 × F2) | | | | |
| | | PLSD | Flx vs Veh | | | <0.05* | |
| | | Post-hoc test | CORT/Flx vs Veh | | | <0.01** | |
| | | | CORT/Veh vs | | | <0.01## | |
| | | | CORT/Flx | | | | |
| Open Field | Time in the | 3-way repeated | Factor 1 Pre- | F = 0.148 | 1, 230 | <0.70 | 4A |
| Paradigm | center | measures ANOVA | treatment | | | | |
| | | | Factor 2 | F = 9.45 | 2, 230 | <0.01** | |
| | | | Treatment | | | | |
| | | | Factor 3 | F = 0.092 | 5, 230 | <0.76 | |
| | | | Time | | | | |
| | | | Interaction | F = 1.27 | 10, 230 | <0.27 | |
| | | | (F1 × F2 × F3) | | | | |
| | | PLSD | SHAM/Flx vs | | | <0.05* | |
| | | Post-hoc test | SHAM/Veh | | | | |
| | | | XRAY/Flx vs | | | <0.05* | |
| | | | SHAM/Veh | | | | |
| | Total Time in | 2-way ANOVA | Factor 1- Pre- | F = 0.148 | 1, 48 | <0.7 | 4B |
| | the center | | treatment | | | | |
| | | | Factor 2 | F = 9.45 | 1, 46 | <0.05* | |
| | | | Treatment | | | | |
| | | | Interaction | F = 0.092 | 1, 46 | <0.76 | |
| | | | (F1 × F2) | | | | |
| | | PLSD | SHAM/Flx vs | | | <0.05* | |
| | | Post-hoc test | SHAM/veh | | | | |
| | | | XRAY/Flx vs | | | <0.05* | |
| | | | SHAM/Veh | | | | |
| | | | XRAY/Flx vs | | | <0.01## | |
| | | | XRAY/Veh | | | | |
| | Entries in the | 2-way ANOVA | Factor 1- Pre- | F = 1.245 | 1, 46 | <0.27 | 4C |
| | enter | | treatment | | | | |
| | | | Factor 2 | F = 2.682 | 1, 46 | <0.10 | |
| | | | Treatment | | | | |
| | | | Interaction | F = 0.067 | 1, 46 | <0.79 | |
| | | | (F1 × F2) | | | | |
| | | Planned | SHAM/Flx vs | | | <0.01** | |
| | | comparisons test | sham/Veh | | | | |
| | | | XRAY/Flx vs | | | <0.05* | |
| | | | SHAM/Veh | | | | |
| | Ambulatory | 2-way ANOVA | Factor 1- Pre- | F = 0.01 | 1, 46 | <0.92 | 4D |
| | Distance in the | | treatment | | | | |
| | center/Total | | Factor 2 | F = 45.56 | 1, 46 | <0.01** | |
| | distance | | Treatment | | | | |
| | | | Interaction | F = 1.55 | 1, 46 | <0.21 | |
| | | | (F1 × F2) | | | | |
| | | PLSD | SHAM/Flx vs | | | <0.01** | |
| | | Post-hoc test | SHAM/veh | | | | |
| | | | XRAY/Flx vs | | | <0.01** | |
| | | | SHAM/Veh | | | | |
| | | | XRAY/Flx vs | | | <0.01## | |
| | | | XRAY/Veh | | | | |
| Novelty | Latency to | 2-way ANOVA | Factor 1- Pre- | F = 1.64 | 1, 49 | <0.2 | 4E |
| Suppressed | feed | | treatment | | | | |
| Feeding | | | Factor 2 | F = 2.69 | 1, 49 | <0.10 | |
| | | | Treatment | | | | |
| | | | Interaction | F = 6.82 | 1, 49 | <0.01** | |
| | | | (F1 × F2) | | | | |
| | | PLSD | SHAM/Flx vs | | | <0.01** | |
| | | Post-hoc test | SHAM/veh | | | | |
| | | Kaplan-Meier | | | | <0.10 | 4G |
| | | survival analysis | | | | | |
| | Food | 2-way ANOVA | Factor 1- Pre- | F = 0.37 | 1, 49 | <0.54 | 4F |
| | consumption | | treatment | | | | |
| | | | Factor 2 | F = 1.74 | 1, 49 | <0.19 | |
| | | | Treatment | | | | |
| | | | Interaction | F = 0.016 | 1, 49 | <0.89 | |
| | | | (F1 × F2) | | | | |
| The Forced | Immobility | 2-way ANOVA | Factor 1- Pre- | F = 0.061 | 1, 49 | <0.8 | 4H |
| Swim test | duration | | treatment | | | | |
| | | | Factor 2 | F = 25.66 | 1, 49 | <0.01** | |
| | | | Treatment | | | | |
| | | | Interaction | F = 0.11 | 1, 49 | <0.9 | |
| | | | (F1 × F2) | | | | |
| | | PLSD | SHAM/Flx vs | | | <0.01** | |
| | | Post-hoc test | SHAM/veh | | | | |
| | | | XRAY/Flx vs | | | <0.01** | |
| | | | SHAM/Veh | | | | |
| Gene | β-arrestin 1 | One-way ANOVA | | F = 3.59 | 3, 27 | <0.01** | 5A |
| expression in | | Newman-Keuls | CORT vs Veh | | | <0.05* | |
| the | | Post-hoc test | CORT/Flx vs | | | <0.05# | |
| hypothalamus | | | CORT/veh | | | | |
| | β-arrestin 2 | One-way ANOVA | | F = 3.61 | 3, 22 | <0.05 | SB |
| | | Newman-Keuls | CORT/Flx vs | | | <0.05# | |
| | | Post-hoc test t | CORT/veh | | | | |
| | Gᵢα2 | One-way ANOVA | | F = 3.88 | 3, 27 | <0.01** | 5C |
| | | Newman-Keuls | CORT vs Veh | | | <0.05* | |
| | | Post-hoc test | CORT/Flx vs | | | <0.05# | |
| | | | CORT/veh | | | | |
| Gene | β-arrestin 1 | One-way ANOVA | | F = 3.02 | 3, 27 | <0.01** | 5D |
| expression in | | Newman-Keuls | CORT vs Veh | | | >0.051 | |
| the amygdala | | Post-hoc test | | | | | |
| | β-arrestin 2 | One-way ANOVA | | F = 3.04 | 3, 21 | >0.051 | 5D |
| | Gᵢα2 | One-way ANOVA | | F = 4.88 | | <0.01** | 5E |
| | | Newman-Keuls | CORT vs veh | | | <0.05* | |
| | | Post-hoc test | CORT/Flx vs /veh | | | <0.05* | |
| Gene | β-arrestin 1 | One-way ANOVA | | F = 2.20 | 3, 27 | >0.05 | 5F |
| expression in | β-arrestin 2 | One-way ANOVA | | F = 3.09 | 3, 27 | >0.051 | 5H |
| the | | Newman-Keuls | CORT/Flx vs veh | | | <0.05* | |
| hippocampus | | Post-hoc test | | | | | |
| | Gᵢα2 | One-way ANOVA | | F = 2.61 | | >0.05 | 5I |
| Open Field | Time in the | 3-way repeated | Factor 1-Pre- | F = 8.76 | 1, 295 | <0.01** | 6A |
| Paradigm | center | measures ANOVA | treatment | | | | |
| | | | Factor 2 | F = 1.50 | 1, 295 | >0.22 | |
| | | | Treatment | | | | |
| | | | Factor 3 | F = 14.79 | 5, 295 | <0.01** | |
| | | | Time | | | | |
| | | | Interaction | F = 0.80 | 5, 390 | >0.52 | |
| | | | (F1 × F2 × F3) | | | | |
| | | PLSD | WT/Flx vs | | | <0.01** | |
| | | Post-hoc test | WT/Veh, t15 | | | | |
| | | | CORT vs | | | <0.01** | |
| | | | CORT/Flx, t30 | | | | |
| | Total Time in | 2-way ANOVA | Factor 1″-Pre- | F = 8.76 | 1, 59 | <0.01** | 6B |
| | the center | | treatment | | | | |
| | | | Factor 2 | F = 1.50 | 1, 59 | >0.22 | |
| | | | Treatment | | | | |
| | | | Interaction | F = 2.94 | 1, 59 | <0.09 | |
| | | | (F1 × F2) | | | | |
| | | PLSD | WT/Flx vs | | | <0.05* | |
| | | Post-hoc test | βArr2KO/Flx | | | | |
| | Entries in the | 2-way ANOVA | Factor 1″-Pre- | F = 7.98 | 1, 59 | <0.01** | 6C |
| | enter | | treatment | | | | |
| | | | Factor 2 | F = 0.69 | 1, 59 | >0.40 | |
| | | | Treatment | | | | |
| | | | Interaction | F = 2.73 | 1, 59 | <0.1 | |
| | | | (F1 × F2) | | | | |
| | | PLSD | WT/Flx vs | | | <0.05* | |
| | | Post-hoc test | βArr2KO/Flx | | | | |
| | Ambulatory | 2-way ANOVA | Factor 1″-Pre- | F = 7.17 | 1, 59 | <0.01** | 6D |
| | Distance | | treatment | | | | |
| | | | Factor 2 | F = 0.12 | 1, 59 | >0.72 | |
| | | | Treatment | | | | |
| | | | Interaction | F = 1.58 | 1, 59 | >0.21 | |
| | | | (F1 × F2) | | | | |
| | | PLSD | WT/Flx vs | | | <0.01# | |
| | | Post-hoc test | βArr2KO/Veh | | | | |
| Novelty | Latency to | 2-way ANOVA | Factor 1- Pre- | F = 17.108 | 1, 59 | <0.01** | 6E |
| Suppressed | feed | | treatment | | | | |
| Feeding | | | Factor 2 | F = 4.781 | 1, 59 | <0.05* | |
| | | | Treatment | | | | |
| | | | Interaction | F = 1.749 | 1, 59 | >0.19 | |
| | | | (F1 × F2) | | | | |
| | | PLSD | WT/Veh vs | | | <0.05* | |
| | | Post-hoc test | βArr2KO/Veh | | | | |
| | | | WT/Flx vs | | | <0.01** | |
| | | | βArr2KO/Flx | | | | |
| | | | WT/Veh vs WT/Flx | | | <0.05* | |
| | | | βArr2KO/Veh vs | | | >0.49 | |
| | | | βArr2KO/Flx | | | | |
| | | Kaplan-Meier | | | | <0.01** | 6F |
| | | survival analysis | | | | | |
| | Food | 2-way ANOVA | Factor 1- Pre- | F = 2.82E⁻⁴ | 1, 59 | >0.98 | 6G |
| | consumption | | treatment | | | | |
| | | | Factor 2 | F = 0.008 | 1, 59 | >0.92 | |
| | | | Treatment | | | | |
| | | | Interaction | F = .523 | 1, 59 | >0.47 | |
| | | | (F1 × F2) | | | | |
| The Forced | Immobility | 2-way ANOVA | Factor 1- Pre- | F = 0.136 | 1, 59 | >0.71 | 6H |
| Swim test | duration | | treatment | | | | |
| | | | Factor 2 | F = 8.117 | 1, 59 | <0.01** | |
| | | | Treatment | | | | |
| | | | Interaction | F = 0.484 | 1, 59 | >0.48 | |
| | | | (F1 × F2) | | | | |
| | | PLSD | WT/Veh vs | | | >0.79 | |
| | | Post-hoc test | βArr2KO/Veh | | | | |
| | | | WT/Flx vs | | | >0.50 | |
| | | | βArr2KO/Flx | | | | |
| | | | WT/Veh vs WT/Flx | | | <0.05* | |
| | | | βArr2KO/Veh vs | | | >0.13 | |
| | | | βArr2KO/Flx | | | | |
| Open Field | Time in the | 2-way repeated | Factor 1′-Pre- | F = 2.78 | 2, 210 | <0.05* | S2A |
| Paradigm | center | measures | treatment | | | | |
| | | ANOVA | Factor 2 | F = 4.98 | 5, 230 | <0.01** | |
| | | | Treatment | | | | |
| | | | Interaction | F = 1.87 | 10, 230 | <0.05* | |
| | | | (F1 × F2) | | | | |
| | | PLSD | CORT 35 ug vs Veh | | | <0.05* | |
| | | Post-hoc test | (t5, t10, 115, 120) | | | | |
| | Total Time in | one-way ANOVA | | F = 2.97 | 2, 42 | <0.05* | S2B |
| | the center | PLSD | CORT 35 ug vs Veh | | | <0.01** | |
| | | Post-hoc test | | | | | |
| | Entries in the | one-way ANOVA | | F = 5.98 | 2, 42 | <0.01** | S2C |
| | enter | PLSD | CORT 35 ug vs Veh | CORT | | <0.01** | |
| | | Post-hoc test | | 35 ug vs | | | |
| | | | | Veh | | | |
| | Total | one-way ANOVA | | F = 4.26 | 2, 42 | <0.01** | S2D |
| | ambulatory | PLSD | CORT 35 ug vs | | | <0.05# | |
| | distance | Post-hoc test | CORT 7 ug | | | | |
| Novelty | Latency to | one-way ANOVA | | F = 4.01 | 2, 42 | <0.05* | S2E |
| Suppressed | feed | PLSD | CORT 7 ug vs Veh | | 2, 42 | <0.01** | |
| Feeding | | Post-hoc test | CORT 35 ug vs Veh | | 2, 42 | <0.01** | |
| | | PLSD | SHAM/Flx vs | | | <0.01** | |
| | | Post-hoc test | SHAM/veh | | | | |
| | | Kaplan-Meier | | | | <0.01** | S2G |
| | | survival analysis | | | | | |
| | Food | one-way ANOVA | | F = 1.52 | 2, 42 | <0.23 | S2F |
| | consumption | | | | | | |
| The Forced | Immobility | one-way ANOVA | | F = 1.59 | 2, 42 | <0.21 | S2H |
| Swim test | duration | | | | | | |
| Mouse body | | 2-way repeated | Factor 1- Pre- | F = 5.65 | 1, 232 | <0.01** | S3A |
| weight | | measures ANOVA | treatment | | | | |
| | | | Factor 2 | F = 205.88 | 4, 232 | <0.01** | |
| | | | Time | | | | |
| | | | Interaction | F = 47.67 | 4, 232 | <0.01** | |
| | | | (F1 × F2) | | | | |
| | | PLSD | CORT wk3 vs Veh | | | <0.01** | |
| | | Post-hoc test | wk3 | | | | |
| | | | CORT wk4 v Veh | | | <0.01** | |
| | | | wk4 | | | | |
| Food | | 2-way repeated | Factor 1- Pre- | F = 6.21 | 1, 16 | <0.01** | S3B |
| consumption | | measures ANOVA | treatment | | | | |
| | | | Factor 2 | F = 2.55 | 4, 16 | <0.01** | |
| | | | Time | | | | |
| | | | Interaction | F = 1.60 | 4, 232 | <0.01** | |
| | | | (F1 × F2) | | | | |
| | | PLSD | CORT wk2 v Veh | | | <0.01** | |
| | | Post-hoc test | wk2 | | | | |
| | | | CORT wk3 v Veh | | | <0.01** | |
| | | | wk3 | | | | |
| | | | CORT wk4 v Veh | | | <0.01** | |
| | | | wk4 | | | | |
| Drinking | | 2-way repeated | Factor 1- Pre- | F = 40.8 | 1, 16 | <0.01** | S3C |
| consumption | | measures ANOVA | treatment | | | | |
| | | | Factor 2 | F = 2.25 | 4, 16 | <0.1 | |
| | | | Time | | | | |
| | | | Interaction | F = 4.40 | 4, 232 | <0.01** | |
| | | | (F1 × F2) | | | | |
| | | PLSD | CORT wkt v Veh | | | <0.01** | |
| | | Post-hoc test | wk1 | | | | |
| | | | CORT wk2 v Veh | | | <0.01** | |
| | | | wk42 | | | | |
| | | | CORT wk3 v Vehwk3 | | | <0.01** | |
| | | | CORT wk4 v Veh | | | <0.01** | |
| | | | wk4 | | | | |
| Home cage | Ratio | PLSD | Unpaired -test | t = 2.817 | 1 | <0.05* | S4A |
| activity | ambulatory | Post-hoc test | | | | | |
| | distance during | | | | | | |
| | the dark phase | | | | | | |
| | over the light | | | | | | |
| | phase | | | | | | |
| | Ambulatory | PLSD | Unpaired -test | t = 5.45 | 1, 7 | <0.01** | S4B |
| | distance during | Post-hoc test | | | | | |
| | the dark phase | | | | | | |
| | Ambulatory | PLSD | Unpaired -test | t = 1.62 | 1, 7 | <0.14 | S4C |
| | distance during | Post-hoc test | | | | | |
| | the light phase | | | | | | |
| | Ambulatory | 2-way ANOVA | Factor 1- Pre- | F = 11.83 | 1, 7 | <0.01** | S4D |
| | distance | | treatment | | | | |
| | | | Factor 2 | F = 0.65 | 1, 7 | <0.44 | |
| | | | Treatment | | | | |
| | | | Interaction | F = 0.031 | 1, 7 | <0.86 | |
| | | | (F1 × F2) | | | | |
| | | PLSD | CORT vs Veh | | | <0.01** | |
| | | Post-hoc test | CORT/Flx vs Veh | | | <0.01** | |
| | Inactivity | 2-wayANOVA | Factor 1- Pre- | F = 55.6 | 1, 7 | <0.01** | S4E |
| | duration | | treatment | | | | |
| | | | Factor 2 | F = 0.25 | 1, 7 | <0.6 | |
| | | | Treatment | | | | |
| | | | Interaction | F = 0.089 | 1, 7 | <0.72 | |
| | | | (F1 × F2) | | | | |
| | | PLSD | CORT vs Veh | | | <0.01** | |
| | | Post-hoc test | CORT/Flx vs Veh | | | <0.01** | |
| Open Field | Time in the | 2-way repeated | Factor 1′- | F = 5.75 | 1, 130 | <0.05* | S5A |
| Paradigm | center | measures | Treatment | | | | |
| | | ANOVA | Factor 2 | F = 8.45 | 5, 130 | <0.01** | |
| | | | Time | | | | |
| | | | Interaction | F = 1.83 | 5, 130 | <0.11 | |
| | | | (F1 × F2) | | | | |
| | | PLSD | CORT 35 ug vs Veh | | | <0.01** | |
| | | Post-hoc test | (t20, t25, t30) | | | | |
| | Total Time in | PLSD | Unpaired t-test | T = 2.398 | | <0.05* | S5B |
| | the center | Post-hoc test | | | | | |
| | Entries in the | PLSD | Unpaired t-test | T = 2.66 | | <0.05* | S5C |
| | enter | Post-hoc test | | | | | |
| | Total | PLSD | Unpaired t-test | T = −1.50 | | <0.14 | S5D |
| | ambulatory | Post-hoc test | | | | | |
| | distance | | | | | | |
| Novelty | Latency to | PLSD | Unpaired t-test | T = −2.13 | | <0.05* | S5E |
| Suppressed | feed | Post-hoc test | | | | | |
| Feeding | | Kaplan-Meier | | | | <0.05* | S5G |
| | | survival analysis | | | | | |
| | Food | one-way ANOVA | | F = 1.34 | | <0.19 | S5F |
| | consumption | | | | | | |
| The Forced | Immobility | PLSD | Unpaired t-test | T = −0.614 | | <0.54 | S5H |
| Swim test | duration | Post-hoc test | | | | | |
| Gene | MR receptor | One-way ANOVA | | F = 2.75 | 3, 27 | >0.05 | S6A |
| expression in | Creb1 | One-way ANOVA | | F = 2.25 | 3, 27 | >0.05 | S6B |
| the | | | | | | | |
| hypothalamus | | | | | | | |
| Gene | MR receptor | One-way ANOVA | | F = 0.33 | 3, 27 | >0.05 | S6C |
| expression in | Creb1 | One-way ANOVA | | F = 0.26 | 3, 27 | >0.05 | S6D |
| the amygdala | | | | | | | |
| Gene | MR receptor | One-way ANOVA | | F = 2.15 | 3, 27 | >0.05 | S6E |
| expression in | Creb1 | One-way ANOVA | | F = 0.17 | 3, 27 | >0.05 | S6F |
| the | | | | | | | |
| hippocampus | | | | | | | |

Factor 1 - pretreatment: Vehicle or Corticosterone; Factor 1′- pretreatment: SHAM or XRAY; Factor 1″- pretreatment: SHAM or XRAY
Factor 2- treatment: Vehicle, fluoxetine, imipramine
Legend: CORT: corticosterone; Imi; imipramine; Flx: fluoxetine; MR: mineralocorticoid receptor; WT: wild-type; βArr2KO: β-Arrestin 2 Knock Out mice

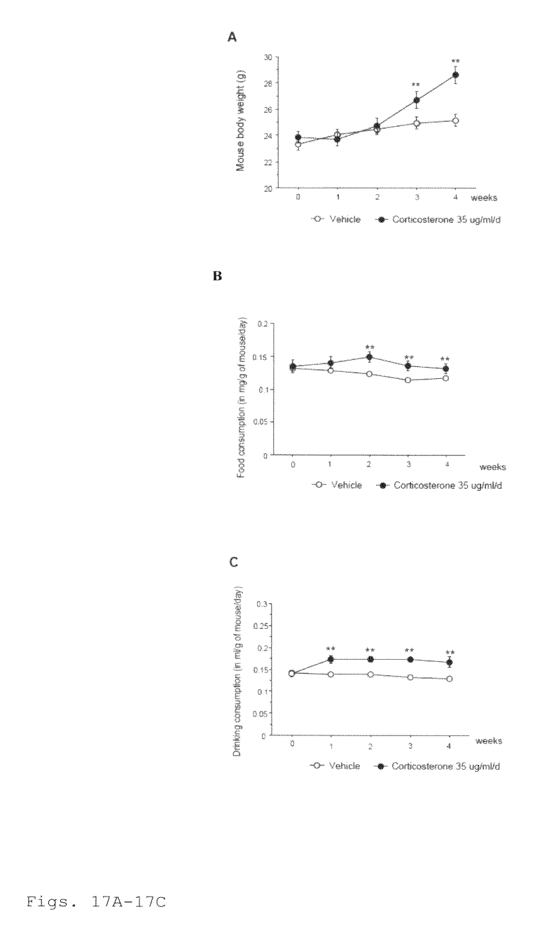

### Experimental Procedures for FIGS. 15-20

### Subjects

For all the experiments, adult male C57BL/6Ntac mice CD1 mice were purchased from Taconic Farms (Germantown, N.Y., USA; Lille Skensved, Denmark) and Jackson Laboratories (Bar Harbor, USA) respectively. All mice were 7-8 weeks old and weighed 23-35 g at the beginning of the treatment, and were maintained on a 12 L:12 D schedule (lights on at 0600) and housed in groups of five of the same strain. Food and water were provided ad libitum. Behavioral testing occurred during the light phase between 0700 and 1900 for the OF, NSF and FST, splash test.

### Behavioral Testing

Mouse body weight Mouse body weight for each animal was followed once a week during the 4-weeks of corticosterone treatment. Food consumption Food consumption was followed once a week during the 4-weeks of corticosterone treatment in each cage. Drinking consumption Drinking consumption was followed once a week during the 4-weeks of corticosterone treatment in each cage. Home cage activity Home cage activity was quantified using the ActiV-Meter (Bioseb, France) over a 24 hours period. During the experiment, food and water were provided ad libitum. Various parameters such as activity time (sec), ambulatory distance (cm) and inactivity duration (calculated from the difference between immobility and motionless activity duration while the animal is eating or scratching) were recorded. The open field paradigm, the novelty suppressed feeding, the forced swim test The procedure for each behavioral test, i.e. the open field paradigm, the novelty suppressed feeding, the forced swim test, is described in the materials and methods section.

### Gene Analysis

Tissue preparation, RNA extraction, DNA preparation and qPCR analysis were described in the materials and methods section. Data analysis and statistics Results from data analyses were expressed as mean±SEM. Data were analyzed using StatView 5.0 software (SAS Institute, Cary, N.C.). For all experiments one-way or two-way ANOVA with repeated measure were applied to the data as appropriate. Significant main effects and/or interactions were resolved followed by Fisher's protected least significant difference (PLSD) post hoc ANOVAs analysis or post hoc unpaired t-tests as appropriate. In the NSF test, the Kaplan-Meier survival analysis was also used because of the lack of normal distribution of the data. Animals that did not eat during the 10 min testing period were censored. Mantel-Cox log-rank test was used to evaluate differences between experimental groups.

### Third Series of Experiments

More than half of depressed patients do not respond to their first drug treatment, and the reasons for this treatment resistance remain enigmatic. Recent data from human studies suggest that high levels of the serotonin-1A (5-HT_{1A}) autoreceptor may correlate with an increased susceptibility to depression and poor treatment response. Here a novel transgenic mouse model is disclosed to directly test the involvement of 5-HT_{1A} autoreceptors in depression-related behavior and the response to antidepressants. Here it is demonstrated that mice with high levels of 5-HT_{1A} autoreceptor are more susceptible to behavioral despair. Moreover, while mice with high levels of 5-HT_{1A} autoreceptor are resistant to treatment with the antidepressant (AD) fluoxetine, a reduction of 5-HT_{1A} autoreceptor levels is sufficient to confer treatment responsiveness. These results establish a causal relationship between 5-HT_{1A} autoreceptor levels, depression, and the response to antidepressants.

Depression is one of the leading public health problems in the world today and antidepressants are amongst the most commonly prescribed medications. However, fewer than half of patients respond to their first drug treatment (A. J. Rush et al., Am J Psychiatry 163, 1905 (Nov., 2006)), and current AD drugs have a delayed onset of action of between 3 and 6 weeks. Together, this results in prolonged pain and suffering and increased medical costs. Therefore, elucidating the mechanisms underlying treatment resistance and the delayed onset of action of AD drugs remains an important and unmet need.

Nearly all antidepressants target the serotonergic system, including the most commonly used, selective serotonin reuptake inhibitors (SSRIs). Serotonin is released solely from serotonergic neurons, which have cell bodies localized in the mid-brain raphe nuclei but send axonal projections all over the brain. Thus SSRIs increase extracellular serotonin throughout the brain, impacting a diverse group of serotonin receptors. While the exact subset and location of receptors responsible for clinical efficacy is not clear, pre-clinical and clinical evidence implicate the 5-HT1A receptor (5-HT1AR) in both the etiology of depression and in the response to treatment (B. Le Francois, M. Czesak, D. Steubl, P. R. Albert, Neuropharmacology, (Jun. 29, 2008)).

Studying the 5-HT1AR is complicated by the fact that it exists as two distinct functional populations in the brain: an inhibitory autoreceptor expressed by serotonergic neurons in the raphe nuclei, and an inhibitory heteroreceptor in non-serotonergic neurons in the rest of the brain. Thus, while 5-HT1A autoreceptors directly participate in negative feedback regulation of raphe firing and set overall serotonergic tone in the brain (P. Blier, G. Pineyro, M. el Mansari, R. Bergeron, C. de Montigny, Ann N Y Acad Sci 861, 204 (Dec. 15, 1998)), 5-HT1A heteroreceptors directly mediate some of the responses to released serotonin. Negative feedback from 5-HT1A autoreceptors is hypothesized to contribute to the delayed therapeutic action of antidepressant drugs by limiting the initial increase in serotonin in the brain. (A. M. Gardier, I. Malagie, A. C. Trillat, C. Jacquot, F. Artigas, Fundam Clin Pharmacol 10, 16 (1996)); the role of the 5-HT1A heteroreceptors to antidepressant drugs is less clear.

Studies in conventional knockout (KO) mice suggest that 5-HT_{1A}Rs are generally involved in modulating both anxiety and depressive-like behavior (L. K. Heisler et al., Proc Natl Acad Sci USA 95, 15049 (Dec. 8, 1998); C. L. Parks, P. S. Robinson, E. Sibille, T. Shenk, M. Toth, Proc Natl Acad Sci USA 95, 10734 (Sep. 1, 1998); S. Ramboz et al., Proc Natl Acad Sci USA 95, 14476 (Nov. 24, 1998)). Mice lacking 5-HT_{1A}Rs throughout life display decreased behavioral despair in response to stress, while displaying a robust and reproducible anxiety-like phenotype as assessed in conflict-anxiety paradigms such as the Open Field (OF) and Light/Dark choice (L/D) test. Anxiety disorders and other stress related disorders such as depression are often co-morbid in humans, and SSRIs are efficacious in treating both. Thus, the combination of an anxious phenotype with a decreased immobility in response to forced swim stress (FST) in 5-HT_{1A}R KO mice is seemingly paradoxical, and much rodent research has focused on the role of 5-HT_{1A}Rs in anxiety while largely ignoring any link with stress or depression.

In contrast, data from human studies suggest a link between 5-HT1A and depression or the response to antidepressants, with less data supporting a role in trait anxiety and anxiety disorders (A. Strobel et al., J Neural Transm 110, 1445 (Dec., 2003)). Most recently, an association has been reported between a C(−1019)G polymorphism in the promoter region of the Htr1a gene and both depression and response to Ads (B. Le Francois, M. Czesak, D. Steubl, P. R. Albert, Neuropharmacology, (Jun. 29, 2008)). Specifically, individuals with the G/G genotype are more susceptible to depression and less responsive to AD treatment, while individuals with the C/C genotype are more resistant to developing depression and more responsive to treatment when they do become depressed. In vitro work in raphe-derived cells suggests that the G allele is less responsive to transcriptional suppression than the C-allele (S. Lemonde et al., J Neurosci 23, 8788 (Sep. 24, 2003)). This has led to the prediction that G/G carriers have higher levels of 5-HT1A autoreceptors, and that C/C carriers have lower levels of 5-HT1A autoreceptors. This prediction fits well with the model that more 5-HT1A autoinhibition is associated with depression and a poor or slower response to AD treatment.

Although consistent with existing models, the putative association of 5-HT1A autoreceptor levels with depression and treatment responsiveness is based on indirect and correlational data. Animal models capable of establishing a direct causal relationship between the 5-HT1A autoreceptor levels, depression and response to antidepressants have remained elusive. Pharmacological approaches have been hampered by the difficulty in separating effects on autoreceptors from effects on heteroreceptors. To directly test the relationship between 5-HT_{1A} autoreceptor levels and anxiety, depression, and the response to antidepressant treatment, we generated mice in which 5-HT_{1A} autoreceptor levels can be specifically and reversibly modulated without affecting heteroreceptor levels.

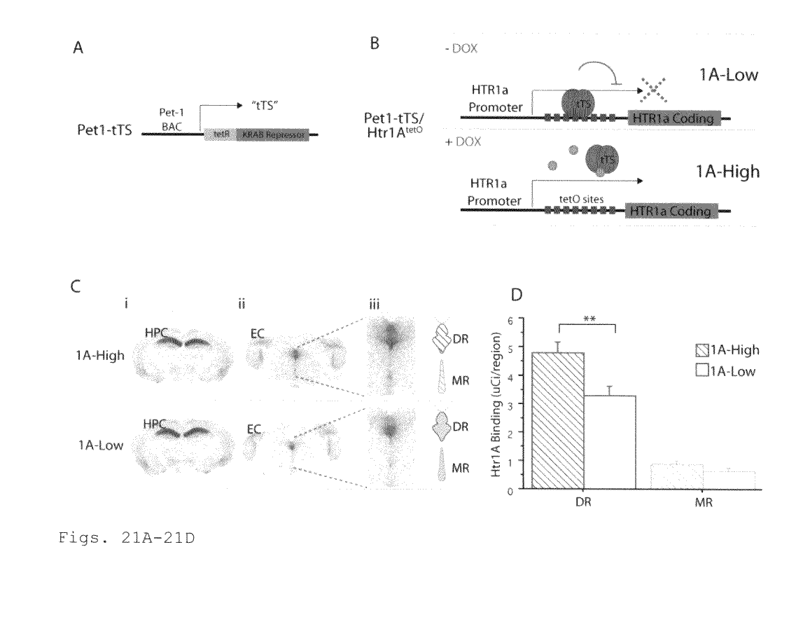

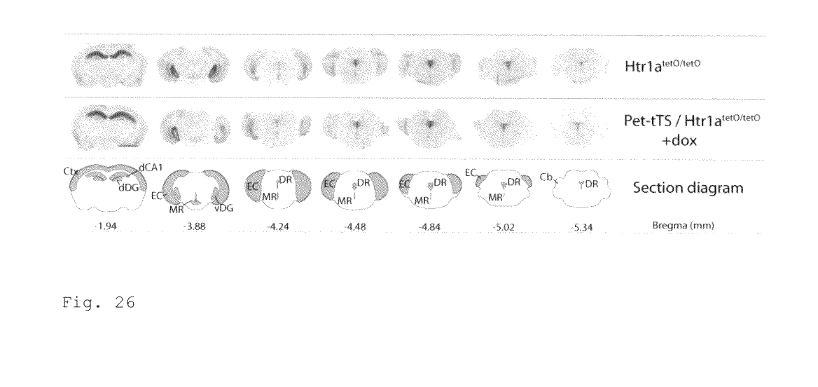

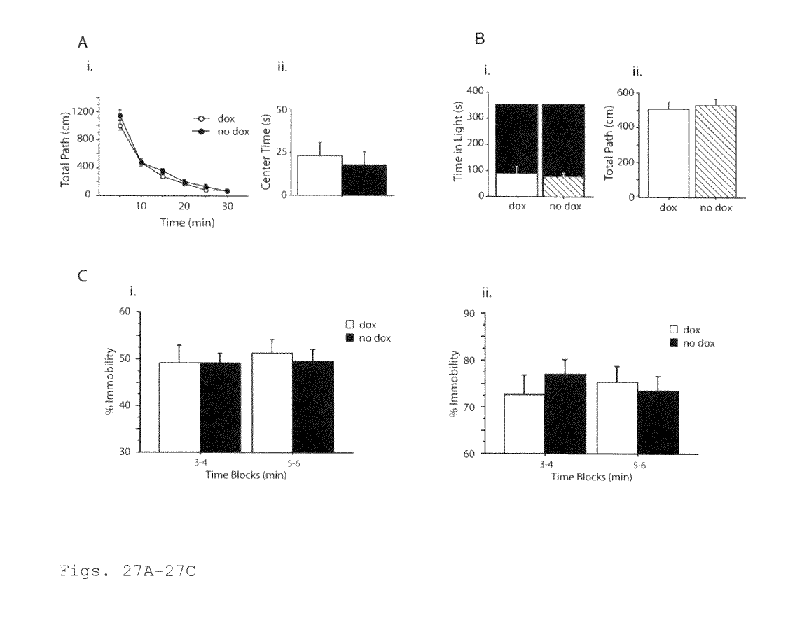

This was accomplished using a novel bigenic system consisting of two parts: 1) insertion of the tet0 DNA regulatory element into the promoter region of the Htr1a gene, to create the tet0-1A allele and 2) raphe-specific expression of the tetracycline-dependent transcriptional suppressor (tTS) under the control of the previously characterized 540Z Pet-1 promoter fragment (P. M. Fisher et al., Nat Neurosci 9, 1362 (Nov., 2006)). Insertion of the tet0 element into the endogenous Htr1a locus does not interfere with normal 5-HT_{1A}R expression patterns, and tTS reversibly suppresses endogenous expression in the raphe by binding to tet0 (FIG. 21a) (M. Mallo, B. Kanzler, S. Ohnemus, Genomics 81, 356 (Apr., 2003)). Mice homozygous for the tet0-1A allele and possessing one copy of the Pet-tTS transgene are fully de-repressed and have high levels of 5-HT_{1A} autoreceptor that are indistinguishable from littermates lacking the tTS transgene (1A-High) (FIG. 26). Removal of doxycycline for four weeks beginning at postnatal day (PND) 50 creates an adult population of animals with lower expression of 5-HT_{1A} autoreceptors (1A-Low) (FIG. 21b). Four weeks after doxycycline removal, suppression has reached maximal levels (data not shown). To control for the possible effect of doxycycline on behavior, all behavioral measures were also assessed in transgene-negative littermates, and no effect of doxycycline was observed in any of the measures presented (FIG. 27).

Quantitative autoradiography revealed that, compared to fully de-repressed mice, the 1A-Low mice show indistinguishable levels of 5-HT_{1A} heteroreceptor expression, but display autoreceptor expression at about 30% below the levels of 1A-High mice (FIG. 21c). The most pronounced differences are seen in the dorsal raphe, while lesser differences are observed in the median raphe, (FIG. 21d). An overall difference of 30% in autoreceptor levels is similar to the difference seen in raphe-derived cell lines expressing reporter constructs containing the human C(−1019)G polymorphic alleles (S. Lemonde et al., J Neurosci 23, 8788 (Sep. 24, 2003)).

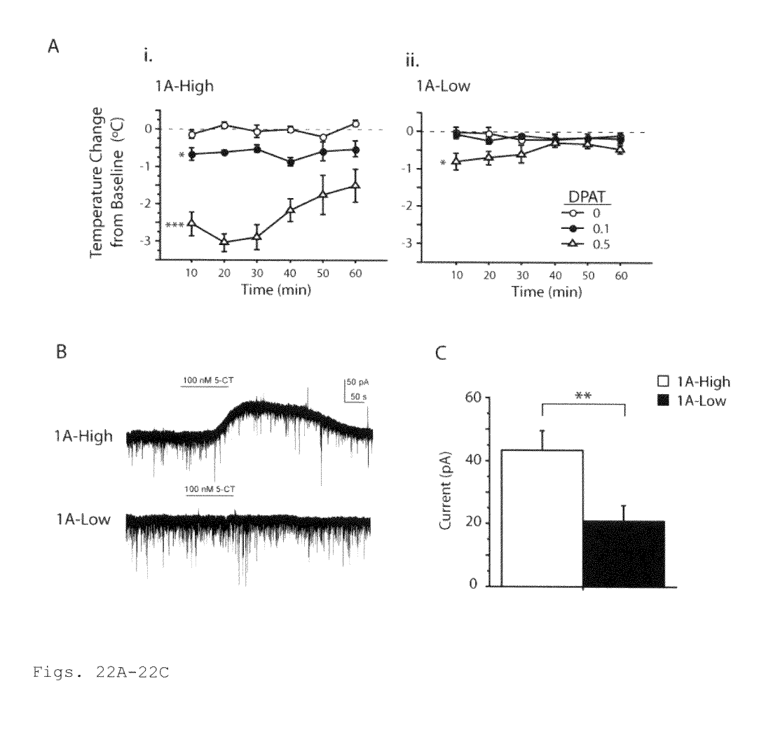

To determine whether the observed differences in 5-HT_{1A} receptor expression levels in the raphe neurons of our transgenic lines had a physiological effect, the hypothermic response to a 5-HT_{1A} agonist challenge was examined. While 1A-High mice displayed a robust and dose-dependent hypothermic response to the 5-HT_{1A} agonist 8-OHDPAT, 1A-Low mice displayed an attenuated response, which was detected only at the highest dose (FIG. 22a). This result is consistent with significant differences in 5-HT_{1A} autoreceptor-mediated signaling between 1A-High and 1A-Low mice.

To directly confirm the differences in 5-HT_{1A} autoreceptors, whole cell recordings were performed in the dorsal raphe and measured the response to the 5-HT_{1A} agonist 5-CT. A significantly higher average current elicited by agonist challenge was observed in the serotonergic neurons of 1A-High mice vs. 1A-Low mice (FIGS. 22B,C). Much of this difference is accounted for by a smaller proportion of serotonergic neurons responding to the agonist challenge in the 1A-Low mice. These data suggest that the tTS mediated transcriptional suppression in the 1A-Low mice results in a mosaic population of serotonergic neurons, some of which retain full responsiveness to 5-HT_{1A}, agonists while others are no longer responsive. Overall, this results in decreased auto-inhibition in the 1A-Low mice relative to the 1A-High mice.

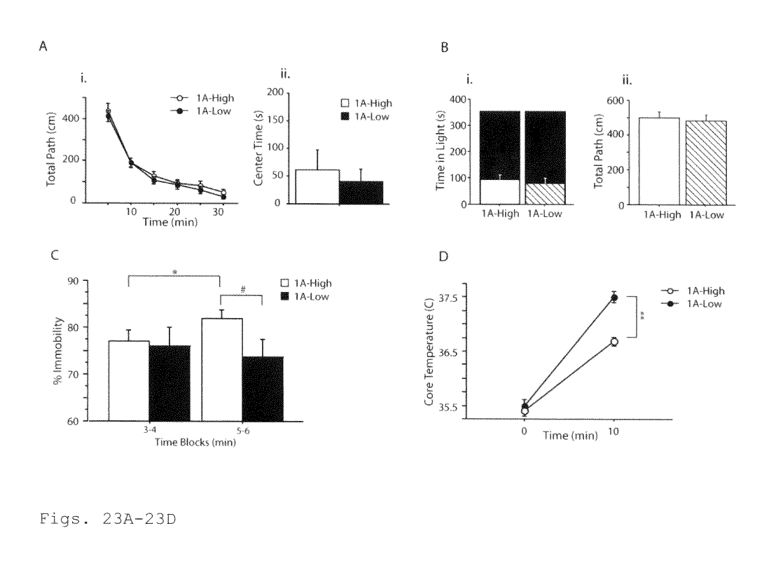

To test whether specifically modulating 5-HT_{1A}-mediated autoinhibition in adulthood impacts anxiety-like behavior, the behavior of the mice was tested in two conflict based tests: the OF paradigm, and the L/D test. 1A-High and 1A-Low mice displayed no difference in either total exploration or exploration in the center of the OF (FIG. 23a). Similarly, in the L/D test, no difference was detected between the groups in total exploration or in the amount of time spent in the light compartment (FIG. 23b). These finding are consistent with previous data implicating the heteroreceptor, but not the autoreceptor, in anxiety-like behavior (C. Gross et al., Nature 416, 396 (Mar. 28, 2002)). Furthermore, the absence of anxiety-like differences in the mice is also consistent with the paucity of evidence linking anxiety disorders to either allele of the human C(−1019)G polymorphism.

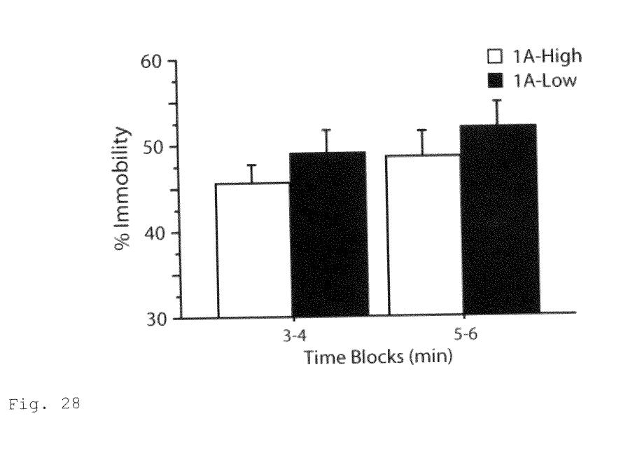

In contrast, human studies do suggest that 5-HT_{1A} autoreceptor levels might influence behavioral resilience to stressful situations, with putative high-expressers being more susceptible to depression than putative low-expressers (S. Lemonde et al., J Neurosci 23, 8788 (Sep. 24, 2003); S. Anttila et al., J Neural Transm 114, 1065 (2007); M. R. Kraus et al., Gastroenterology 132, 1279 (Apr., 2007); C. D. Neff et al., Mol Psychiatry, (Feb. 12, 2008)). To directly test whether modulating 5-HT_{1A} autoinhibition in adulthood impacts behavioral responsiveness to stress, we subjected our mice to inescapable swim stress in the FST. Animals were exposed to the stressor twice over a 24-hour period, and immobility was scored as a measure of behavioral despair (I. Lucki, Behav Pharmacol 8, 523 (Nov., 1997)). While 1A-High and 1A-Low mice responded indistinguishably to the initial stressor (FIG. 28), 1A-High mice displayed progressively more immobility, or behavioral despair, upon re-exposure the second day compared to 1A-Low mice (FIG. 23c), suggesting a different adaptation to stress in the two groups. These results are consistent not only with previously reported behavior in traditional 5-HT_{1A} KO mice, but also with human genetic data suggesting a link between high 5-HT_{1A} autoreceptor levels and increased susceptibility to depression.

To further confirm that specific modulation of serotonergic autoinhibition alters stress responsivity, the response of the 1A-High and 1A-Low mice was examined in the stress induced hyperthermia paradigm (SIH). In this paradigm, animals are placed in a novel cage for ten minutes, and the increase in body temperature from baseline is assayed as a measure of autonomic reactivity to stress. In this test, the 1A-High mice show a blunted autonomic response to an acute stressor compared to the 1A-Low animals (FIG. 23d). This difference in autonomic reactivity may contribute to the more passive coping strategy adopted by the 1A-High mice in the FST.

Having demonstrated that a modest change in serotonergic autoinhibition yielded a consistent difference in responsiveness to stress, it was next asked whether such a change also contributed to the responsiveness to antidepressant drugs. Human studies suggest that, in addition to modulating susceptibility to depression, 5-HT_{1A} autoreceptor levels are also associated with response to Ads (S. Lemonde, L. Du, D. Bakish, P. Hrdina, P. R. Albert, Int Neuropsychopharmacol 7, 501 (Dec., 2004); C. C. Meltzer et al., Neuropsychopharmacology 29, 2258 (Dec., 2004)). To directly test whether the response to AD treatment is affected by autoreceptor levels, we treated 1A-High and 1A-Low mice with either vehicle or the SSRI fluoxetine and tested behavioral response in the well-established novelty-suppressed feeding (NSF) paradigm (S. R. Bodnoff, B. Suranyi-Cadotte, D. H. Aitken, R. Quirion, M. J. Meaney, Psychopharmacology (Berl) 95, 298 (1988); C. Gross, L. Santarelli, D. Brunner, X. Zhuang, R. Hen, Biol Psychiatry 48, 1157 (Dec. 15, 2000); L. Santarelli et al., Science 301, 805 (Aug. 8, 2003)). The NSF paradigm is a test of hyponeophagia that measures the latency of a mouse to consume food placed in the middle of a brightly lit, aversive arena. It has two features which make it useful to model the human response to antidepressants: 1) latency to eat decreases in response to chronic, but not acute, treatment with antidepressant drugs, and 2) similarly to other behavioral tests of antidepressant response, some mouse strains respond in this paradigm, while others do not (I. Lucki, A. Dalvi, A. J. Mayorga, Psychopharmacology (Berl) 155, 315 (May, 2001)) and data not shown). Thus, unlike behavioral tests in which mice respond to acute treatment with antidepressants (such as the tail-suspension test or the FST), the NSF provides a model that closely resembles the human response to antidepressants (S. C. Dulawa, R. Hen, Neurosci Biobehav Rev 29, 771 (2005); A. Lira et al., Biol Psychiatry 54, 960 (Nov. 15, 2003)).

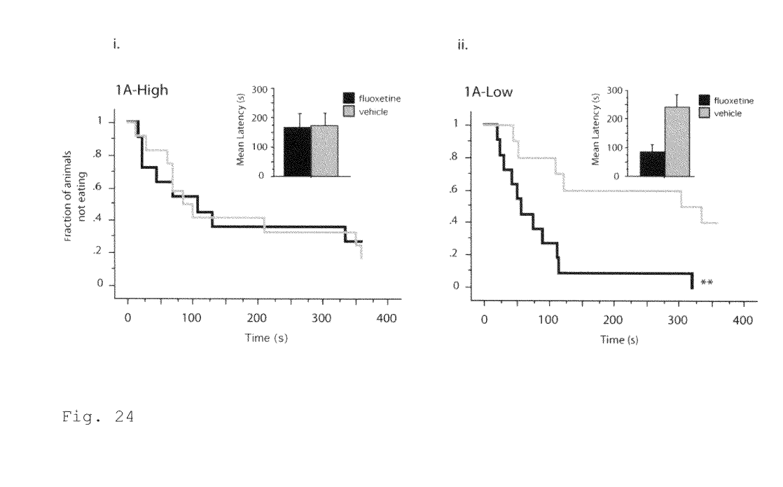

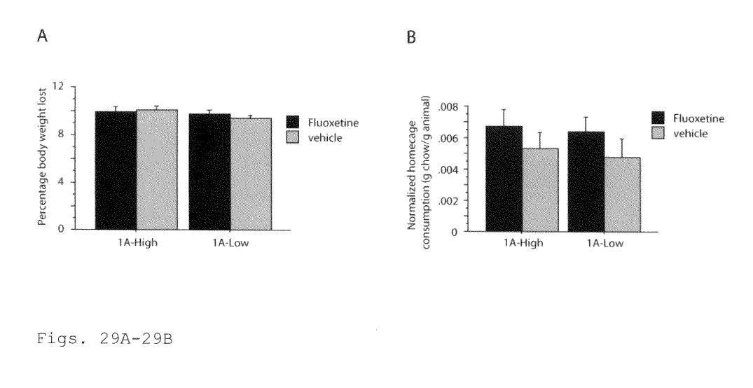

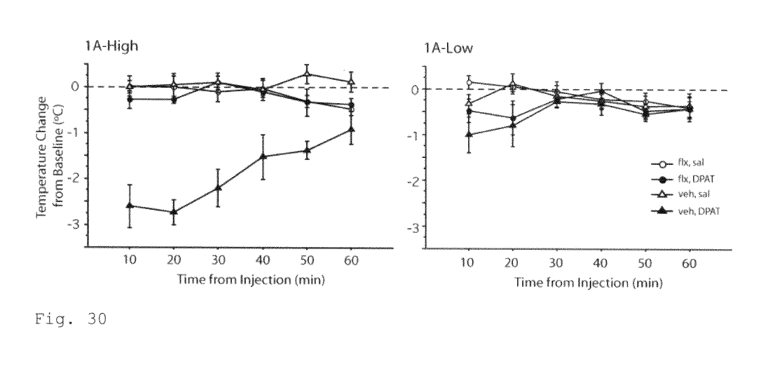

Following twenty-five days of treatment with fluoxetine, 1A-Low mice responded robustly in the NSF, as evidenced by their lower latency to feed relative to their vehicle treated controls; conversely, no response to fluoxetine was observed in the 1A-High mice (FIG. 24b). Results were not confounded by motivational differences between the groups (FIG. 29). This experiment establishes a causal relationship between 5-HT_{1A} autoreceptor levels and response to ADs; namely, a modest change in 5-HT_{1A} autoreceptor levels in adulthood is sufficient to confer responsiveness to fluoxetine in an otherwise treatment-resistant population. Finally, the failure of 1A-High mice to respond to chronic fluoxetine treatment was not due to a lack of autoreceptor desensitization at the time of testing (FIG. 30), suggesting that desensitization of autoreceptors alone is not sufficient for response, but rather that 5-HT_{1A}-mediated serotonergic tone prior to treatment is critical for establishing treatment response.

In conclusion, the data presented here address the role of the 5-HT_{1A} autoreceptor in both baseline measures of anxiety and stress responsiveness, and in the response to antidepressants. First, this study establishes a double dissociation of 5-HT_{1A}R function in baseline measures, both between autoreceptors and heteroreceptors, and between development and adulthood. Previous work has suggested that developmental expression of 5-HT_{1A} heteroreceptors is sufficient to establish normal anxiety-like behavior, regardless of 5-HT_{1A}R status at the time of testing (C. Gross et al., Nature 416, 396 (Mar. 28, 2002)). The data presented here demonstrates the complementary point: specific manipulation of 5-HT_{1A} autoreceptors in adulthood is sufficient to impact depression-related behavior and autonomic reactivity to stress without affecting conflict-anxiety behavioral measures. Secondly, this study establishes the first causal link between 5-HT_{1A} autoreceptor levels and responsiveness to antidepressant. This study is the first to demonstrate that specific modulation of 5-HT_{1A} autoreceptors in adulthood is sufficient to confer responsiveness to antidepressant treatment in an otherwise treatment-resistant population.

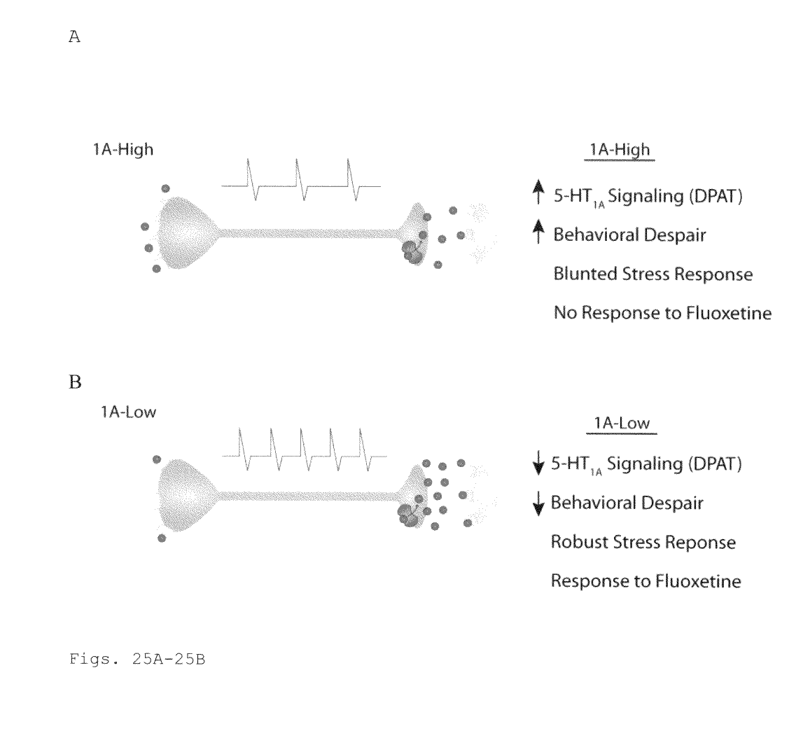

Overall, the data presented here provide direct evidence supporting a model in which intrinsic raphe firing rates are directly related to resilience under stress and to the response to antidepressant treatment (FIG. 25). In such a model, higher intrinsic 5-HT_{1A} autoreceptor levels result in lower basal firing rates of the raphe, while lower intrinsic 5-HT_{1A} autoreceptor levels result in higher basal firing rates. This higher basal raphe firing rate makes an animal more resilient to stress, consistent with a previous association between high serotonin levels and low stress susceptibility. Upon treatment with an SSRI, 5-HT_{1A} autoreceptor-mediated negative feedback inhibits raphe firing. We expect this occurs similarly in both 1A-High and 1A-Low animals. With time, increased serotonin causes autoreceptors to desensitize, returning the raphe to its original high or low basal firing rates in the 1A-Low or 1A-High animals, respectively. Because serotonin reuptake is blocked in both cases, the predicted result is a larger increase in serotonin levels in the chronically treated 1A-Low animals, as evidenced by their behavioral response to treatment.

### REFERENCES

Airan R D, Meltzer L A, Roy M, Gong Y, Chen H, Deisseroth K (2007). High-speed imaging reveals neurophysiological links to behavior in an animal model of depression. Science 317:819-823.
American Psychiatric Association (1990). The practice of ECT: recommendations for treatment, training and privileging. Convuls Ther 6:85-120.
Anderson G M, Barr C S, Lindell S, Durham A C, Shifrovich I, Higley J D (2005). Time course of the effects of the serotonin-selective reuptake inhibitor sertraline on central and peripheral serotonin neurochemistry in the rhesus monkey. Psychopharmacology (Berl) 178:339-346.
Bodnoff S R, Suranyi-Cadotte B, Quirion R, Meaney M J (1989). A comparison of the effects of diazepam versus several typical and atypical anti-depressant drugs in an animal model of anxiety. Psychopharmacology (Berl) 97:277-279.
Brown J, Cooper-Kuhn C M, Kempermann G, Van Praag H, Winkler J, Gage F H, Kuhn H G (2003). Enriched environment and physical activity stimulate hippocampal but not olfactory bulb neurogenesis. Eur J Neurosci 17:2042-2046.
Brown R E, Reymann K G (1995). Metabotropic glutamate receptor agonists reduce paired-pulse depression in the dentate gyrus of the rat in vitro. Neurosci Lett 196:17-20.
Castrén E, Võikar V, Rantamäki T (2007). Role of neurotrophic factors in depression. Curr Opin Pharmacol 7:18-21.
Chen Z Y, Jing D, Bath K G, Ieraci A, Khan T, Siao C J, Herrera D G, Toth M, Yang C, McEwen B S, Hempstead B L, Lee F S (2006). Genetic variant BDNF (Val66Met) polymorphism alters anxiety-related behavior. Science 314:140-143.
Couillard-Despres S, Winner B, Schaubeck S, Aigner R, Vroemen M, Weidner N, Bogdahn U, Winkler J, Kuhn H G, Aigner L (2005). Doublecortin expression levels in adult brain reflect neurogenesis. Eur J Neurosci 21:1-14.
DeLean A, Munson P J, Rodbard D (1978). Simultaneous analysis of families of sigmoidal curves: application to bioassay, radioligand assay, and physiological dose-response curves. Am J Physiol 235:E97-E102.
Encinas J M, Vaahtokari A, Enikolopov G (2006). Fluoxetine targets early progenitor cells in the adult brain. Proc Natl Acad Sci USA 103:8233-8238.
Fujioka T, Fujioka A, Duman R S (2004). Activation of cAMP signaling facilitates the morphological maturation of newborn neurons in adult hippocampus. J Neurosci 24:319-328.
Ge S, Goh E L, Sailor K A, Kitabatake Y, Ming G L, Song H (2006) GABA regulates synaptic integration of newly generated neurons in the adult brain. Nature 439:589-593.

Ge S, Yang C H, Hsu K S, Ming G L, Song H (2007). A critical period for enhanced synaptic plasticity in newly generated neurons of the adult brain. Neuron 54:559-566.

Kempermann G, Kuhn H G, Gage F H (1997). More hippocampal neurons in adult mice living in an enriched environment. Nature 386:493-495.

Kreiss D S, Lucki I (1995). Effects of acute and repeated administration of antidepressant drugs on extracellular levels of 5-hydroxytryptamine measured in vivo. J Pharmacol Exp Ther 274:866-876.
Madsen T M, Treschow A, Bengzon J, Bolwig T G, Lindvall O, Tingstrom A (2000). Increased neurogenesis in a model of electroconvulsive therapy. Biol Psychiatry 47:1043-1049.
Malberg J E, Eisch A J, Nestler E J, Duman R S (2000). Chronic antidepressant treatment increases neurogenesis in adult rat hippocampus. J Neurosci 20:9104-9110.
McNaughton B L (1980). Evidence for two physiologically distinct perforant pathways to the fascia dentata. Brain Res 199:1-19.
Mennerick S, Zorumski C F (1995). Paired-pulse modulation of fast excitatory synaptic currents in microcultures of rat hippocampal neurons. J Physiol (Lond) 488:85-101.
Meshi D, Drew M R, Saxe M, Ansorge M S, David D, Santarelli L, Malapani C, Moore H, Hen R (2006). Hippocampal neurogenesis is not required for behavioral effects of environmental enrichment. Nat Neurosci 9:729-731.
Millan M J (2006). Multi-target strategies for the improved treatment of depressive states: conceptual foundations and neuronal substrates, drug discovery and therapeutic application. Pharmacol Ther 110:135-370.
Nakagawa S, Kim J E, Lee R, Malberg J E, Chen J, Steffen C, Zhang Y J, Nestler E J, Duman R S (2002). Regulation of neurogenesis in adult mouse hippocampus by cAMP and the cAMP response element-binding protein. J Neurosci 22:3673-3682.
Overstreet-Wadiche L S, Bromberg D A, Bensen A L, Westbrook G L (2006). Seizures accelerate functional integration of adult-generated granule cells. J Neurosci 26:4095-4103.
Plumpe T, Ehninger D, Steiner B, Klempin F, Jessberger S, Brandt M, Romer B, Rodriguez G R, Kronenberg G, Kempermann G (2006). Variability of doublecortin-associated dendrite maturation in adult hippocampal neurogenesis is independent of the regulation of precursor cell proliferation. BMC Neurosci 7:77.
Radley J J, Jacobs B L (2003). Pilocarpine-induced status epilepticus increases cell proliferation in the dentate gyrus of adult rats via a 5-HT1A receptor-dependent mechanism. Brain Res 966:1-12.
Rantamäki T, Hendolin P, Kankaanpää A, Mijatovic J, Piepponen P, Domenici E, Chao M V, Männistö PT, Castrén E (2007). Pharmacologically diverse antidepressants rapidly activate brain-derived neurotrophic factor receptor TrkB and induce phospholipase-Cgamma signaling pathways in mouse brain. Neuropsychopharmacology 32:2152-2162.

Rutter J J, Gundlah C, Auerbach S B (1994). Increase in extracellular serotonin produced by uptake inhibitors is enhanced after chronic treatment with fluoxetine. Neurosci Lett 171:183-186.
Sackeim H A, Luber B, Katzman G P, Moeller J R, Prudic J, Devanand D P, Nobler M S (1996). The effects of electroconvulsive therapy on quantitative electroencephalograms. Relationship to clinical outcome. Arch Gen Psychiatry 53:814-824.
Santarelli L, Saxe M, Gross C, Surget A, Battaglia F, Dulawa S, Weisstaub N, Lee J, Duman R, Arancio O, Belzung C, Hen R (2003). Requirement of hippocampal neurogenesis for the behavioral effects of antidepressants. Science 301:805-809.
Saxe M D, Battaglia F, Wang J W, Malleret G, David D J, Monckton J E, Garcia A D, Sofroniew M V, Kandel E R, Santarelli L, Hen R, Drew M R (2006). Ablation of hippocampal neurogenesis impairs contextual fear conditioning and synaptic plasticity in the dentate gyrus. Proc Natl Acad Sci USA 103:17501-17506.
Schmidt-Hieber C, Jonas P, Bischofberger J (2004). Enhanced synaptic plasticity in newly generated granule cells of the adult hippocampus. Nature 429:184-187.
Snyder J S, Kee N, Wojtowicz J M (2001). Effects of adult neurogenesis on synaptic plasticity in the rat dentate gyrus. J Neurophysiol 85:2423-2431.
Stewart C A, Reid I C (2000). Repeated ECS and fluoxetine administration have equivalent effects on hippocampal synaptic plasticity. Psychopharmacology (Berl) 148:217-223.
Suckow R F, Zhang M F, Cooper T B (1992). Sensitive and selective liquid-chromatographic assay of fluoxetine and norfluoxetine in plasma with fluorescence detection after precolumn derivatization. Clin Chem 38:1756-1761.
van Praag H, Shubert T, Zhao C, Gage F H (2005). Exercise enhances learning and hippocampal neurogenesis in aged mice. J Neurosci 25:8680-8685.
Wang S, Scott B W, Wojtowicz J M (2000). Heterogenous properties of dentate granule neurons in the adult rat. J Neurobiol 42:248-257.
Warner-Schmidt J L, Duman R S (2007). VEGF is an essential mediator of the neurogenic and behavioral actions of antidepressants. Proc Natl Acad Sci USA 104:4647-4652.
Wong M L, Licinio J (2001). Research and treatment approaches to depression. Nat Rev Neurosci 2:343-351.
Alonso, R., Griebel, G., Pavone, G., Stemmelin, J., Le Fur, G., and Soubrie, P. (2004). Blockade of CRF(1) or V(1b) receptors reverses stress-induced suppression of neurogenesis in a mouse model of depression. Molecular psychiatry 9, 278-286, 224.
Antonijevic, I. A. (2006). Depressive disorders—is it time to endorse different pathophysiologies? Psychoneuroendocrinology 31, 1-15. Ardayfio, P., and Kim, K. S. (2006). Anxiogenic-like effect of chronic corticosterone in the light-dark emergence task in mice. Behav Neurosci 120, 249-256.
Artigas, F., Romero, L., de Montigny, C., and Blier, P. (1996). Acceleration of the effect of selected antidepressant drugs in major depression by 5-HT1A antagonists. Trends in neurosciences 19, 378-383.
Avissar, S., Matuzany-Ruban, A., Tzukert, K., and Schreiber, G. (2004). Betaβ-arrestin-1 levels: reduced in leukocytes of patients with depression and elevated by antidepressants in rat brain. Am J Psychiatry 161, 2066-2072.
Avissar, S., Nechamkin, Y., Roitman, G., and Schreiber, G. (1998). Dynamics of ECT normalization of low G protein function and immunoreactivity in mononuclear leukocytes of patients with major depression. Am J Psychiatry 155, 666-671.
Beaulieu, J. M., Marion, S., Rodriguiz, R. M., Medvedev, I. O., Sotnikova, T. D., Ghisi, V., Wetsel, W. C., Lefkowitz, R. J., Gainetdinov, R. R., and Caron, M. G. (2008). A betaarrestin 2 signaling complex mediates lithium action on behavior. Cell 132, 125-136.
Beaulieu, J. M., Sotnikova, T. D., Marion, S., Lefkowitz, R. J., Gainetdinov, R. R., and Caron, M. G. (2005). An Akt/betaβ-arrestin 2/PP2A signaling complex mediates dopaminergic neurotransmission and behavior. Cell 122, 261-273.
Calfa, G., Kademian, S., Ceschin, D., Vega, G., Rabinovich, G. A., and Volosin, M. (2003). Characterization and functional significance of glucocorticoid receptors in patients with major depression: modulation by antidepressant treatment. Psychoneuroendocrinology 28, 687-701.
Conrad, C. D., McLaughlin, K. J., Harman, J. S., Foltz, C., Wieczorek, L., Lightner, E., and Wright, R. L. (2007). Chronic glucocorticoids increase hippocampal vulnerability to neurotoxicity under conditions that produce CA3 dendritic retraction but fail to impair spatial recognition memory. J Neurosci 27, 8278-8285.
David, D. J., Klemenhagen, K. C., Holick, K. A., Saxe, M. D., Mendez, I., Santarelli, L., Craig, D. A., Zhong, H., Swanson, C. J., Hegde, L. G., et al. (2007). Efficacy of the MCHR1 antagonist N-[3-(1-{[4-(3,4-difluorophenoxy)phenyl]methyl}(4-piperidyl))-4-methylphen yl]-2-methylpropanamide (SNAP 94847) in mouse models of anxiety and depression following acute and chronic administration is independent of hippocampal neurogenesis. J Pharmacol Exp Ther 321, 237-248.
de Kloet, E. R., Joels, M., and Holsboer, F. (2005). Stress and the brain: from adaptation to disease. Nature reviews 6, 463-475.
Dulawa, S. C., Holick, K. A., Gundersen, B., and Hen, R. (2004). Effects of chronic fluoxetine in animal models of anxiety and depression. Neuropsychopharmacology 29, 1321-1330.
Duman, R. S., Malberg, J., Nakagawa, S., and D'Sa, C. (2000). Neuronal plasticity and survival in mood disorders. Biological psychiatry 48, 732-739.
Dwivedi, Y., Rizavi, H. S., Conley, R. R., Roberts, R. C., Tamminga, C. A., and Pandey, G. N. (2002). mRNA and protein expression of selective alpha subunits of G proteins are abnormal in prefrontal cortex of suicide victims. Neuropsychopharmacology 27, 499-517.
Franklin, K. B., and Paxinos, G. (1997). The Mouse Brain in Stereotaxic Coorfinates (Toronto/San Diego: Academic Press).
Gainetdinov, R. R., Premont, R. T., Bohn, L. M., Lefkowitz, R. J., and Caron, M. G. (2004). Desensitization of G protein-coupled receptors and neuronal functions. Annual review of neuroscience 27, 107-144.
Gould, E., Cameron, H. A., Daniels, D. C., Woolley, C. S., and McEwen, B. S. (1992). Adrenal hormones suppress cell division in the adult rat dentate gyrus. J Neurosci 12, 3642-3650.
Gould, E., Tanapat, P., McEwen, B. S., Flugge, G., and Fuchs, E. (1998). Proliferation of granule cell precursors in the dentate gyrus of adult monkeys is diminished by stress. Proceedings of the National Academy of Sciences of the United States of America 95, 3168-3171.
Gourley, S. L., Wu, F. J., Kiraly, D. D., Ploski, J. E., Kedves, A. T., Duman, R. S., and Taylor, J. R. (2008). Regionally specific regulation of ERK MAP kinase in a model of antidepressant-sensitive chronic depression. Biological psychiatry 63, 353-359.
Greden, J. F., Gardner, R., King, D., Grunhaus, L., Carroll, B. J., and Kronfol, Z. (1983). Dexamethasone suppression tests in antidepressant treatment of melancholia. The process of normalization and test-retest reproducibility. Archives of general psychiatry 40, 493-500.
Griebel, G., Simiand, J., Serradeil-Le Gal, C., Wagnon, J., Pascal, M., Scatton, B., Maffrand, J. P., and Soubrie, P. (2002). Anxiolytic- and antidepressant-like effects of the non-peptide vasopressin V1b receptor antagonist, SSR149415, suggest an innovative approach for the treatment of stress-related disorders. Proceedings of the National Academy of Sciences of the United States of America 99, 6370-6375.
Grippo, A. J., Sullivan, N. R., Damjanoska, K. J., Crane, J. W., Carrasco, G. A., Shi, J., Chen, Z., Garcia, F., Muma, N. A., and Van de Kar, L. D. (2005). Chronic mild stress induces behavioral and physiological changes, and may alter serotonin 1A receptor function, in male and cycling female rats. Psychopharmacology (Berl) 179, 769-780.
Heine, V. M., Maslam, S., Joels, M., and Lucassen, P. J. (2004). Prominent decline of newborn cell proliferation, differentiation, and apoptosis in the aging dentate gyrus, in absence of an age-related hypothalamus-pituitary-adrenal axis activation. Neurobiol Aging 25, 361-375.
Hensler, J. G., Advani, T., and Monteggia, L. M. (2007). Regulation of serotonin-1A receptor function in inducible brain-derived neurotrophic factor knockout mice after administration of corticosterone. Biological psychiatry 62, 521-529.
Heuser, I. J., Schweiger, U., Gotthardt, U., Schmider, J., Lammers, C. H., Dettling, M., Yassouridis, A., and Holsboer, F. (1996). Pituitary-adrenal-system regulation and psychopathology during amitriptyline treatment in elderly depressed patients and normal comparison subjects. Am J Psychiatry 153, 93-99.
Holick, K. A., Lee, D. C., Hen, R., and Dulawa, S. C. (2008). Behavioral effects of chronic fluoxetine in BALB/cJ mice do not require adult hippocampal neurogenesis or the serotonin 1A receptor. Neuropsychopharmacology 33, 406-417.
Holsboer, F. (2008). How can we realize the promise of personalized antidepressant medicines? Nature reviews 9, 638-646.
Holsboer-Trachsler, E., Stohler, R., and Hatzinger, M. (1991). Repeated administration of the combined dexamethasone-human corticotropin releasing hormone stimulation test during treatment of depression. Psychiatry Res 38, 163-171.
Joels, M. (2008). Functional actions of corticosteroids in the hippocampus. European journal of pharmacology 583, 312-321.
Koch, J. M., Kell, S., Hinze-Selch, D., and Aldenhoff, J. B. (2002). Changes in CREBphosphorylation during recovery from major depression. Journal of psychiatric research 36, 369-375.
Lefkowitz, R. J., Rajagopal, K., and Whalen, E. J. (2006). New roles for betaβ-arrestins in cell signaling: not just for seven-transmembrane receptors. Molecular cell 24, 643-652.
Linkowski, P., Mendlewicz, J., Kerkhofs, M., Leclercq, R., Golstein, J., Brasseur, M., Copinschi, G., and Van Cauter, E. (1987). 24-hour profiles of adrenocorticotropin, cortisol, and growth hormone in major depressive illness: effect of antidepressant treatment. J Clin Endocrinol Metab 65, 141-152.
Matuzany-Ruban, A., Avissar, S., and Schreiber, G. (2005). Dynamics of betaβ-arrestin1 protein and mRNA levels elevation by antidepressants in mononuclear leukocytes of patients with depression. Journal of affective disorders 88, 307-312.
Mayberg, H. S., Lozano, A. M., Voon, V., McNeely, H. E., Seminowicz, D., Hamani, C., Schwalb, J. M., and Kennedy, S. H. (2005). Deep brain stimulation for treatment-resistant depression. Neuron 45, 651-660.
McEwen, B. S. (1999). Stress and hippocampal plasticity. Annual review of neuroscience 22, 105-122.
McEwen, B. S. (2004). Protection and damage from acute and chronic stress: allostasis and allostatic overload and relevance to the pathophysiology of psychiatric disorders. Annals of the New York Academy of Sciences 1032, 1-7.
McEwen, B. S., and Magarinos, A. M. (2001). Stress and hippocampal plasticity: implications for the pathophysiology of affective disorders. Human psychopharmacology 16, S7-S19.
Morgan, V. A., Mitchell, P. B., and Jablensky, A. V. (2005). The epidemiology of bipolar disorder: sociodemographic, disability and service utilization data from the Australian National Study of Low Prevalence (Psychotic) Disorders. Bipolar disorders 7, 326-337.
Murray, F., Smith, D. W., and Hutson, P. H. (2008). Chronic low dose corticosterone exposure decreased hippocampal cell proliferation, volume and induced anxiety and depression like behaviours in mice. European journal of pharmacology 583, 115-127.
Navailles, S., Hof, P. R., and Schmauss, C. (2008). Antidepressant drug-induced stimulation of mouse hippocampal neurogenesis is age-dependent and altered by early life stress. The Journal of comparative neurology 509, 372-381.
Nemeroff, C. B., and Owens, M. J. (2004). Pharmacologic differences among the SSRIs: focus on monoamine transporters and the HPA axis. CNS spectrums 9, 23-31.
Oakley, R. H., Olivares-Reyes, J. A., Hudson, C. C., Flores-Vega, F., Dautzenberg, F. M., and Hauger, R. L. (2007). Carboxyl-terminal and intracellular loop sites for CRF1 receptor phosphorylation and betaβ-arrestin-2 recruitment: a mechanism regulating stress and anxiety responses. American journal of physiology 293, R209-222.
Perlis, R. H., Purcell, S., Fava, M., Fagerness, J., Rush, A. J., Trivedi, M. H., and Smoller, J. W. (2007).
Association between treatment-emergent suicidal ideation with citalopram and polymorphisms near cyclic adenosine monophosphate response element binding protein in the STAR*D study. Archives of general psychiatry 64, 689-697.
Pierce, K. L., and Lefkowitz, R. J. (2001). Classical and new roles of betaβ-arrestins in the regulation of G-protein-coupled receptors. Nature reviews 2, 727-733. Popa, D., Lena, C., Alexandre, C., and Adrien, J. (2008). Lasting syndrome of depression produced by reduction in serotonin uptake during postnatal development: evidence from sleep, stress, and behavior. J Neurosci 28, 3546-3554.
Porsolt, R. D., Bertin, A., and Jalfre, M. (1977). Behavioral despair in mice: a primary screening test for antidepressants. Archives internationales de pharmacodynamie et de therapie 229, 327-336.
Qiu, G., Helmeste, D. M., Samaranayake, A. N., Lau, W. M., Lee, T. M., Tang, S. W., and So, K. F. (2007). Modulation of the suppressive effect of corticosterone on adult rat hippocampal cell proliferation by paroxetine. Neurosci Bull 23, 131-136.
Schauwecker, P. E. (2006). Genetic influence on neurogenesis in the dentate gyrus of two strains of adult mice. Brain Res 1120, 83-92.
Schreiber, G., and Avissar, S. (2007). Regulators of G-protein-coupled receptor-Gprotein coupling: antidepressants mechanism of action. Expert review of neurotherapeutics 7, 75-84.
Sheline, Y. I. (1996). Hippocampal atrophy in major depression: a result of depression induced neurotoxicity? Molecular psychiatry, 298-299.
Stone, E. A., and Lin, Y. (2008). An anti-immobility effect of exogenous corticosterone in mice. European journal of pharmacology 580, 135-142.
Stout, S. C., Owens, M. J., and Nemeroff, C. B. (2002). Regulation of corticotropinreleasing factor neuronal systems and hypothalamic-pituitary-adrenal axis activity by stress and chronic antidepressant treatment. J Pharmacol Exp Ther 300, 1085-1092.
Surget, A., Saxe, M., Leman, S., Ibarguen-Vargas, Y., Chalon, S., Griebel, G., Hen, R., and Belzung, C. (2008). Drug-Dependent Requirement of Hippocampal Neurogenesis in a Model of Depression and of Antidepressant Reversal. Biological psychiatry.
Tanapat, P., Hastings, N. B., Rydel, T. A., Galea, L. A., and Gould, E. (2001). Exposure to fox odor inhibits cell proliferation in the hippocampus of adult rats via an adrenal hormone-dependent mechanism. The Journal of comparative neurology 437, 496-504.
Wang, J. W., David, D. J., Monckton, J. E., Battaglia, F., and Hen, R. (2008). Chronic fluoxetine stimulates maturation and synaptic plasticity of adult-born hippocampal granule cells. J Neurosci 28, 1374-1384.
Zhao, Y., Ma, R., Shen, J., Su, H., Xing, D., and Du, L. (2008). A mouse model of depression induced by repeated corticosterone injections. European journal of pharmacology 581, 113-120.

1. A method for identifying an agent as an antidepressant or as an anxiolytic comprising:
a) administering the agent to a mammal for a time period of at least 14 days; and
b) determining whether adult-born neurons in the brain of the mammal exhibit (a) increased dendritic arborization, (b) decreased expression of an immaturity marker, (C) increased expression of a maturity marker, or (d) enhanced artificial cerebrospinal fluid-type long-term potentiation (ACSF-LTP) as compared to (a) dendritic arborization, (b) expression of an immaturity marker, (c) expression of a maturity marker, (d) ACSF-LTP, respectively, in a control mammal,
wherein one or more of an increased dendritic arborization, decreased expression of an immaturity marker, increased expression of a maturity marker, or enhanced ACSF-LTP indicates that the agent is an antidepressant or as an anxiolytic.

2. (canceled)

3. A method for identifying an agent as able to increase dendritic arborization, (b) decrease expression of an immaturity marker, (c) increase expression of a maturity marker, or (d) enhance artificial cerebrospinal fluid-type long-term potentiation (ACSF-LTP) in a central nervous system of a mammal comprising:
a) administering the agent to a mammal for a time period of at least 14 days; and
b) determining whether adult-born neurons in the brain of the mammal exhibit (a) increased dendritic arborization, (b) decreased expression of an immaturity marker, (c) increased expression of a maturity marker, or (d) enhanced artificial cerebrospinal fluid-type long-term potentiation (ACSF-LTP) as compared to (a) dendritic arborization, (b) expression of an immaturity marker, (c) expression of a maturity marker, (d) ACSF-LTP, respectively, in a control mammal,
wherein one or more increased dendritic arborization, decreased expression of an immaturity marker, increased expression of a maturity marker, or enhanced ACSF-LTP, indicates that the agent is able to increase dendritic arborization, decrease expression of an immaturity marker, increase expression of a maturity marker, or enhance ACSF-LTP in the central nervous system of the mammal.

4. The method of claim 1, wherein the adult-born neurons are identified as such by their expression of doublecortin.

5. The method of claim 1, wherein the neurons are hippocampal granule cells.

6. The method of claim 1, wherein the dendritic arborization is quantitated by measuring the amount of tertiary branching of the dendrites of the neurons.

7. The method of claim 1, wherein the immaturity marker is doublecortin.

8. The method of claim 1, wherein the time period is at least 28 days.

9. The method of claim 1, wherein in step b) it is determined whether the agent causes increased dendritic arborization.

10. The method of claim 1, wherein in step b) it is determined whether the agent causes a decreased expression of an immaturity marker.

11. The method of claim 1, wherein in step b) it is determined whether the agent causes an increased expression of an immaturity marker.

12. The method of claim 1, wherein in step b) it is determined whether the agent enhances artificial cerebrospinal fluid-type long-term potentiation.

13. A method for identifying an agent as an antidepressant comprising:
a) quantitating (a) dendritic arborization, (b) expression of an immaturity marker, (c) expression of a maturity marker, (d) artificial cerebrospinal fluid-type long-term potentiation ACSF-LTP in mammalian adult-born neurons maintained in culture, or (e) artificial cerebrospinal fluid-type long term potentiation ACSF-LTP in mammalian adult-born neurons of a hippocampal brain slice preparation;
b) contacting the neurons with the agent for a time period of at least 14 days; and
c) determining whether the neurons exhibit (a) increased dendritic arborization, (b) decreased expression of an immaturity marker, (c) increased expression of a maturity marker, or (d) enhanced ACSF-LTP,
wherein increased dendritic arborization, decreased expression of an immaturity marker, increased expression of a maturity marker, or enhanced ACSF-LTP of the mammalian adult born neurons or of the mammalian adult born neurons of the hippocampal brain slice preparation indicates that the agent is an antidepressant.

14.-24. (canceled)

25. A method of identifying whether an agent is an antidepressant or an anxiolytic comprising administering the agent to a mammal and determining if the agent (i) elicits an increase in an amount of beta-arrestin 2 in the brain of the mammal or (ii) activates beta-arrestin 2 in the brain of the mammal, wherein an increase in the amount of beta-arrestin 2 in the brain of the mammal or activation of beta-arrestin 2 in the brain of the mammal indicates that the agent is an antidepressant or an anxiolytic.

26.-35. (canceled)

36. A method of identifying whether an agent is an antidepressant and anxiolytic comprising administering the agent to a mammal and determining if the agent elicits an increase in beta-arrestin levels and Gi.alpha.2 levels in the brain of the mammal, wherein an increase in beta-arrestin levels and Gi.alpha.2 levels in the brain of the mammal indicates that the agent is an antidepressant and anxiolytic.

37.-38. (canceled)

39. A mouse having a depressive phenotype, wherein the depressive phenotype results from administration of a corticosteroid to the mouse, wherein the corticosteroid is administered at a dose of 2-8 ug/kg body mass/day for a period of 14-28 days.

40.-44. (canceled)

45. A transgenic mouse whose genome contains a recombinant DNA sequence comprising: (1) a DNA regulatory element operatively inserted into a promoter of an endogenous DNA sequence which encodes a human 5-hydroxytryptamine1A receptor, and (2) a serotoninergic neuron-specific promoter operatively linked to a DNA sequence encoding a tetracycline-dependent transcriptional suppressor.

46.-54. (canceled)

55. A method for determining whether it is likely an agent can treat an affective disorder in a human having an affective disorder that is resistant to treatment with a selective serotonin reuptake inhibitor, which comprises: (a) quantifying a behavioral parameter which increases with the affective disorder in the transgenic mouse of claim 45, wherein the transgenic mouse exhibits a depressive phenotype that is resistant to treatment with a selective serotonin reuptake inhibitor when the transgenic mouse is fed a tetracycline antibiotic, (b) administering the agent to the mouse and quantifying the behavioral parameter; and (c) determining if the mouse exhibits a lower level of the behavioral parameter in step c) than in step a), wherein if the mouse exhibits a lower level of the behavioral parameter in step c) than in step a) then it is likely that the agent can treat the affective disorder, and wherein if the mouse exhibits a higher level of the behavioral parameter in step c) than in step a) or the same amount of the behavioral parameter in step c) and step a), then it is likely that the agent cannot treat the affective disorder.

56. A method for determining whether it is likely an agent can treat an anxiety disorder in a human having an anxiety disorder that is resistant to treatment with a selective serotonin reuptake inhibitor, which comprises: (a) quantifying a behavioral parameter which increases with the anxiety disorder in the transgenic mouse of claim 45 wherein the transgenic mouse exhibits a depressive phenotype that is resistant to treatment with a selective serotonin reuptake inhibitor when the transgenic mouse is fed a tetracycline antibiotic, (b) administering the agent to the mouse and quantifying the behavioral parameter; and (c) determining if the animal mouse exhibits a lower level of the behavioral parameter in step c) than in step a), wherein if the mouse exhibits a lower level of the behavioral parameter in step c) than in step a) then it is likely that the agent can treat the anxiety disorder, and wherein if the mouse exhibits a higher level of the behavioral parameter in step c) than in step a) or the same amount of the behavioral parameter in step c) and step a), then it is likely that the agent cannot treat the anxiety disorder.

57.-65. (canceled)

66. A method for determining whether it is likely an agent can treat an affective disorder in a human having an affective disorder that is resistant to treatment with a selective serotonin reuptake inhibitor, which comprises: (a) quantifying a behavioral parameter which decreases with the affective disorder in the transgenic mouse of claim 45 wherein the transgenic mouse exhibits a depressive phenotype that is resistant to treatment with a selective serotonin reuptake inhibitor when the transgenic mouse is fed a tetracycline antibiotic, (b) administering the agent to the mouse and quantifying the behavioral parameter; and (c) determining if the mouse exhibits a higher level of the behavioral parameter in step c) than in step a), wherein if the mouse exhibits a higher level of the behavioral parameter in step c) than in step a) then it is likely that the agent can treat the affective disorder, and wherein if the mouse exhibits a lower level of the behavioral parameter in step c) than in step a) or the same amount of the behavioral parameter in step c) and step a), then it is likely that the agent cannot treat the affective disorder.

67. A method for determining whether it is likely an agent can treat an anxiety disorder in a human having an anxiety disorder that is resistant to treatment with a selective serotonin reuptake inhibitor, which comprises: (a) quantifying a behavioral parameter which decreases with the anxiety disorder in the transgenic mouse of claim 45, wherein the transgenic mouse exhibits a depressive phenotype that is resistant to treatment with a selective serotonin reuptake inhibitor when the transgenic mammal is fed a tetracycline antibiotic, (b) administering the agent to the animal and quantifying the behavioral parameter; and (c) determining if the mouse exhibits a higher level of the behavioral parameter in step c) than in step a), wherein if the mouse exhibits a higher level of the behavioral parameter in step c) than in step a) then it is likely that the agent can treat the anxiety disorder, and wherein if the mouse exhibits a lower level of the behavioral parameter in step c) than in step a) or the same amount of the behavioral parameter in step c) and step a), then it is likely that the agent cannot treat the anxiety disorder.


SEQUENCE LISTING (21 sequences)

SEQ ID NO: 1 - DNA, 22 bp, Artificial Sequence
  Primer
ccaccagacagttcctcatgtc

SEQ ID NO: 2 - DNA, 21 bp, Artificial Sequence
  Primer
cattgacgctgatgggttctc

SEQ ID NO: 3 - DNA, 27 bp, Artificial Sequence
  Primer
ccctgcaccttgaggcatctctggata

SEQ ID NO: 4 - DNA, 18 bp, Artificial Sequence
  Primer
tccgctatggccgagaag

SEQ ID NO: 5 - DNA, 20 bp, Artificial Sequence
  Primer
cctggtaggtggcgatgaac

SEQ ID NO: 6 - DNA, 26 bp, Artificial Sequence
  Primer
atgtactgggcctgtctttccgcaaa

SEQ ID NO: 7 - DNA, 20 bp, Artificial Sequence
  Primer
tcaagctgcctctggtgatg

SEQ ID NO: 8 - DNA, 21 bp, Artificial Sequence
  Primer
ggaggacgccataacaactcc

SEQ ID NO: 9 - DNA, 27 bp, Artificial Sequence
  Primer
aaacataccagattcgcacagcaccca

SEQ ID NO: 10 - DNA, 16 bp, Artificial Sequence
  Primer
tttcgccgcttgctgc

SEQ ID NO: 11 - DNA, 17 bp, Artificial Sequence
  Primer
ctcgtcatcggccgtga

SEQ ID NO: 12 - DNA, 25 bp, Artificial Sequence
  Primer
catggtcaaccccaccgtgttcttc

SEQ ID NO: 13 - DNA, 22 bp, Artificial Sequence
  Primer
caaattcaacggcacagtcaag

SEQ ID NO: 14 - DNA, 20 bp, Artificial Sequence
  Primer
accccatttgatgttagtgg

SEQ ID NO: 15 - DNA, 27 bp, Artificial Sequence
  Primer
tcatcaacgggaagcccatcaccatct

SEQ ID NO: 16 - DNA, 19 bp, Artificial Sequence
  Primer
accatggtgtgcaagcctg

SEQ ID NO: 17 - DNA, 23 bp, Artificial Sequence
  Primer
ggtagtaagcggctgagtcattg

SEQ ID NO: 18 - DNA, 23 bp, Artificial Sequence
  Primer
ttggccgctcacgggaatatcaa

SEQ ID NO: 19 - DNA, 22 bp, Artificial Sequence
  Primer
tgtcctcctccacagctagctt

SEQ ID NO: 20 - DNA, 22 bp, Artificial Sequence
  Primer
gcatgtcagtgaggttccttga

SEQ ID NO: 21 - DNA, 26 bp, Artificial Sequence
  Primer
cagtttcccagtgcacagtcccatca